SEQUENCE LISTING (13 sequences)

SEQ ID NO: 1 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 2 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 3 - protein, 5 aa, Artificial Sequence
  Description of Artificial Sequence Synthetic peptide
CPPCP

SEQ ID NO: 4 - protein, 1 aa, Artificial Sequence
  Description of Artificial Sequence Synthetic peptide
G

SEQ ID NO: 5 - protein, 2 aa, Artificial Sequence
  Description of Artificial Sequence Synthetic peptide
GS

SEQ ID NO: 6 - protein, 5 aa, Artificial Sequence
  Description of Artificial Sequence Synthetic peptide
GSGGS

SEQ ID NO: 7 - protein, 5 aa, Artificial Sequence
  Description of Artificial Sequence Synthetic peptide
GGGGS

SEQ ID NO: 8 - protein, 4 aa, Artificial Sequence
  Description of Artificial Sequence Synthetic peptide
GGGS

SEQ ID NO: 9 - protein, 21 aa, Artificial Sequence
  Description of Artificial Sequence Synthetic peptide
SRGGGGSGGGGSGGGGSLEMA

SEQ ID NO: 10 - protein, 7 aa, Artificial Sequence
  Description of Artificial Sequence Synthetic peptide
TSGGGGS

SEQ ID NO: 11 - protein, 18 aa, Artificial Sequence
  Description of Artificial Sequence Synthetic peptide
GEGTSTGSGGSGGSGGAD

SEQ ID NO: 12 - protein, 22 aa, Artificial Sequence
  Description of Artificial Sequence Synthetic peptide
AEAAAKEAAAKEAAAKEAAAKA

SEQ ID NO: 13 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

(19) United States
(12) Patent Application Publication
(10) Pub. No.: US 2022/0235136 A1
(43) Pub. Date: Jul. 28, 2022

(54) METHODS AND COMPOSITIONS FOR TREATING A DISEASE OR DISORDER

(71) Applicants: YALE UNIVERSITY, New Haven, CT (US); The Regents of the University of Colorado, a body corporate, Denver, CO (US)

(72) Inventors: Yuwen ZHU, Parker, CO (US); Lieping CHEN, Hamden, CT (US); Richard SCHULICK, Englewood, CO (US); Yi SUN, Englewood, CO (US)

(73) Assignees: YALE UNIVERSITY, New Haven, CT (US); The Regents of the University of Colorado, a body corporate, Denver, CO (US)

(21) Appl. No.: 17/704,746

(22) Filed: Mar. 25, 2022

Related U.S. Application Data
(63) Continuation of application No. PCT/US2020/052681, filed on Sep. 25, 2020.
(60) Provisional application No. 62/906,282, filed on Sep. 26, 2019.

(51) Int. Cl. C07K 16/28; C07K 16/18; A61K 39/395; A61K 45/06; C07K 14/705; C07K 14/47; A61P 35/00; A61K 31/513; A61K 31/704

(52) U.S. Cl. CPC C07K 16/2851; C07K 16/18; A61K 39/39558; A61K 45/06; A61K 38/00; C07K 14/4743; A61P 35/00; A61K 31/513; A61K 31/704; C07K 14/7056

(57) ABSTRACT

The present application provides agents that specifically inhibits the IGFBP7/CD93 signaling pathway, such as agents that specifically block the interaction between CD93 and IGFBF7, methods of using said agents and methods of identifying said agents.

### CROSS-REFERENCE TO RELATED APPLICATIONS

This application claims priority to U.S. Provisional Application No. 62/906,282, filed Sep. 26, 2019, the disclosure of which is herein incorporated by reference in its entirety.

### SEQUENCE LISTING

The instant application contains a Sequence Listing which has been submitted in ASCII format is EFS-Web and is hereby incorporated by references in its entirety. Said ASCII copy, created on Sep. 22, 2021), is named 251609 000034 SL.txt, and is 11,372 bytes in size.

### FIELD OF THE APPLICATION

The present invention relates to methods and compositions that involve an agent that blocks the CD93/IGFBP7 signaling pathway.

### BACKGROUND

Pathological angiogenesis—driven by an imbalance of pro- and anti-angiogenic signaling is a hallmark of many diseases, both malignant and benign. Unlike in the healthy adult in which angiogenesis is tightly regulated such diseases are characterized by uncontrolled new vessel formation, resulting in a microvascular network characterized by vessel immaturity, with profound structural and functional abnormalities. The consequence of these abnormalities is further modification of the microenvironment, often serving to fuel disease progression and attenuate response to conventional therapies.

Therefore, there is a need for developing methods or compositions for normalizing or promoting the maturation of the vasculature in these diseases (such as cancer).

### BRIEF SUMMARY OF THE APPLICATION

The present application provides methods of treating a tumor (such as a cancer) in a subject in need thereof, comprising administering to the subject an effective amount of a CD93/IGFBP7 blocking agent that specifically inhibits the IGFBP7/CD93 signaling pathway. In some embodiments, the CD93/IGFBP7 blocking agent blocks interaction between CD93 and IGFBP7

In some embodiments, the CD93/IGFBP7 blocking agent comprises an anti-CD93 antibody specifically recognizing CD93. In some embodiments, the anti-CD93 antibody binds to CD93 competitively with mAb MM01 or mAb 7C10. In some embodiments, the anti-CD93 antibody binds to an epitope that overlaps or substantially overlaps with that of mAb MM01 or mAb 7C10. In some embodiments, the anti-CD93 antibody also blocks interaction between CD93 and Multimerin 2 (MMRN2). In some embodiments, the anti-CD93 antibody does not block interaction between CD93 and MMRN2. In some embodiments, the anti-CD93 antibody binds to the IGFBP7 binding site on CD93. In some embodiments, the anti-CD93 antibody binds to a region on CD93 that is outside of the IGFBP7 binding site. In some embodiments, the anti-CD93 antibody binds to an extracellular region of CD93. In some embodiments, the extracellular region of CD93 comprises residues 22-580 of the amino acid sequence of SEQ ID NO: 1. In some embodiments, the anti-CD93 antibody binds to an EGF-like region of CD93. In some embodiments, the EGF-like region of CD93 consists of residues 257-469 and 260-468 of the amino acid sequence of SEQ ID NO: 1. In some embodiments, the anti-CD93 antibody hinds to a C-type lectin domain of CD93. In some embodiments, the C-type lectin domain of CD93 comprises 22-174 of the amino acid sequence of SEQ ID NO: 1. In some embodiments, the anti-CD93 antibody binds to a long-loop region of CD93. In some embodiments, the long-loop region of CD93 comprises residues 96-141 of the amino acid sequence of SEQ ID NO. 1. In some embodiments, the anti-CD93 antibody is an anti-human CD93 antibody. In some embodiments, the anti-human CD93 antibody is mAb MM01 or a humanized version thereof. In some embodiments, the anti-CD93 antibody is a full length antibody, a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, or a tetrabody. In some embodiments, the anti-CD93 is comprised in a fusion protein.

In some embodiments, the CD93/IGFBP7 blocking agent is a polypeptide. In some embodiments, the polypeptide is an inhibitory CD93 polypeptide. In some embodiments, the inhibitory CD93 polypeptide is a fragment of CD93 or a variant of CD93 comprising an extracellular domain of CD93. In some embodiments, the polypeptide is a soluble polypeptide. In some embodiments, the polypeptide is membrane bound. In some embodiments, the inhibitory CD93 polypeptide comprises a variant of the extracellular domain of 193. In some embodiments, the polypeptide binds to IGFBP7 with a greater affinity than for MMNR2. In some embodiments, the polypeptide does not bind to MMNR2. In some embodiments, the polypeptide binds to IGFBP7 with a greater affinity than CD93 does. In some embodiments, the inhibitory CD93 polypeptide comprises a F238 residue, wherein the amino acid numbering is based on SEQ ID NO: 1. In some embodiments, the inhibitory CD93 polypeptide further comprises a stabilizing domain. In some embodiments, the stabilizing domain is an Fc domain. In some embodiments, the polypeptide is about 50 to about 200 amino acids long.

In some embodiments, the CD93/IGFBP7 blocking agent comprises an anti-IGFBP7 antibody specifically recognizing IGFBP7. In some embodiments, the anti-IGFBP7 antibody binds to IGFBP7 competitively with mAb R003 or mAb 2C6. In some embodiments, the anti-IGFBP7 antibody binds to an epitope that overlaps with that of mAb R003 or mAb 2C6. In some embodiments, the anti-IGFBP7 antibody also blocks interaction between IGFBP7 and IGF-1, IGF-2, and/or IGF1R. In some embodiments, the anti-IGFBP7 antibody does not block interaction between IGFBP7 and IGF-1, IGF-2, and, or IGF1R. In some embodiments, the anti-IGFBP7 antibody binds to a CD93 binding site on IGFBP7. In some embodiments, the anti-IGFBP7 antibody binds to a region on IGFBP7 that is outside of the CD93 binding site. In some embodiments, the anti-IGFBP7 antibody binds to an N-terminal domain of IGFBP7 (residues 28-106). In some embodiments, the N-terminal domain of IGFBP7 consists of residues 28-106 of the amino acid sequence of SEQ ID NO: 2. In some embodiments, the anti-IGFBP7 antibody binds to a kazal-like domain of IGFBP7. In some embodiments, the kazal-like domain of IGFBP7 consists of residues 105-158 of the amino acid sequence of SEQ ID NO: 2. In some embodiments, the anti-IGFBP7 antibody binds to the Ig-like C2-type domain of IGFBP7. In some embodiments, the Ig-like C2-type domain of IGFBP7 consists of residues 160-264 of the amino acid sequence of SEQ ID NO: 2. In some embodiments, the anti-IGFBP7 antibody binds to the insulin binding (IB) domain of IGFBP7. In some embodiments, the anti-IGFBP7 antibody is an anti-human IGFBP7 antibody. In some embodiments, the anti-human IGFBP7 antibody is mAb R003 or a humanized version thereof. In some embodiments, the anti-IGFBP7 antibody is a full length antibody, a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, or a tetrabody. In some embodiments, the anti-IGFBP7 antibody is comprised in a fusion protein.

In some embodiments, the CD93/IGFBP7 blocking agent is a polypeptide and the polypeptide is an inhibitory IGFBP7 polypeptide comprising a variant of IGFBP7. In some embodiments, the inhibitory IGFBP7 polypeptide binds to CD93 but does not activate CD93. In some embodiments, the inhibitory IGFBP7 polypeptide binds to 0193 with a greater affinity than for IGF-1, IGF-2, and or IGF1R. In some embodiments, the polypeptide binds to CD93 with a greater affinity than IGFBP7. In some embodiments, the inhibitory IGFBP7 polypeptide comprises the IB domain of IGFBP7. In some embodiments, the inhibitory IGFBP7 polypeptide further comprises a stabilizing domain. In some embodiments, the stabilizing domain is an Fc domain. In some embodiments, the inhibitory IGFBP7 polypeptide is about 50 to about 200 amino acids long.

In some embodiments, the CD93/IGFBP7 blocking agent comprises a fusion protein, a peptide analog, an aptamer, avimer, anticalin, speigelmer, or a small molecule compound.

In some embodiments of any one of the methods described above, the CD93/IGFBP7 blocking agent reduces the expression of CD93 or IGFBP7. In some embodiments, the CD93/IGFBP7 blocking agent comprises a siRNA, a shRNA, a miRNA, an antisense RNA, or a gene editing system.

In some embodiments of any one of the methods described above wherein the method further comprises administering to the subject a second agent. In some embodiments, the second agent is an immune checkpoint inhibitor. In some embodiments, the immune checkpoint inhibitor is selected from the group consisting of an anti-PD1 antibody, an anti-PD-L1 antibody, and an anti-CTLA4 antibody. In some embodiments, the second agent is a chemotherapeutic agent. In some embodiments, the second agent is an immune cell. In some embodiments, the second agent is an anti-angiogenesis inhibitor. In some embodiments, the anti-angiogenesis inhibitor is an anti-VEGF inhibitor.

In some embodiments of any one of the methods described above, the cancer is characterized by abnormal tumor vasculature.

In some embodiments of any one of the methods described above, the cancer is characterized by high expression of VEGF.

In some embodiments of any one of the methods described above, the cancer is characterized by high expression of CD93.

In some embodiments of any one of the methods described above, the cancer is characterized by high expression of IGFBP7.

In some embodiments of any one of the methods described above, the cancer is a solid tumor. In some embodiments, the cancer is colorectal cancer, non-small cell lung cancer, glioblastoma, renal cell carcinoma, cervical cancer, ovarian cancer, fallopian tube cancer, peritoneal cancer, breast cancer, prostate cancer, bladder cancer, oral squamous cell carcinoma, head and neck squamous cell carcinoma, brain tumors, bone cancer, melanoma. In some embodiments, the cancer is enriched with blood vessels. In some embodiments, the cancer is triple-negative breast cancer (TNBC). In some embodiments, the cancer is melanoma. In some embodiments, the patient is resistant to a prior therapy comprising administration of an immune checkpoint inhibitor, e.g., an anti-PD1 antibody, an anti-PD-L1 antibody, an anti-CTLA4 antibody, or a combination thereof. In some embodiments, “enriched” used herein refer to a larger amount or higher density of the blood vessel (e.g., at least 10%, 20%, 30%, 40% or 50% larger or higher) in a tumor tissue as compared to the amount or density of the blood Vessel in a corresponding tissue in a subject that does not have cancer.

In some embodiments, there is also provided methods of determining whether a candidate agent is useful for treating cancer, comprising: determining whether the candidate agent disrupts the CD93/IGFBP7 interaction, wherein the candidate agent is useful for treating cancer if it is shown to specifically disrupt the CD93/IGFBP7 interaction. In some embodiments, the method comprises determining whether the candidate agent disrupts the interaction of CD93 and IGFBP7 on a cell surface. In some embodiments, the method comprises determining whether the candidate agent specifically disrupts interaction CD93 and IGFBP7 in an in vitro assay system. In some embodiments, the in vitro system is a yeast two-hybrid system. In some embodiments, the in vitro system is an ELISA-based assay. In some embodiments, the in vitro system is an FACS-based assay In some embodiments, the candidate agent is an antibody, a peptide, a fusion peptide, a peptide analog, a polypeptide, an aptamer, avimer, anticalin, speigelmer, or a small molecule compound. In some embodiments, the method comprises contacting the candidate agent with a CD93/IGFBP7 complex. In some embodiments, there is provides an agent identified by any of the methods described above.

In some embodiments, there is also provided a non-naturally occurring polypeptide, wherein non-naturally occurring polypeptide is a variant inhibitory CD93 polypeptide comprising the extracellular domain of CD93, wherein the polypeptide blocks interaction between CD93 and IGFBP7. In some embodiments, the variant inhibitory CD93 polypeptide is membrane bound. In some embodiments, the variant inhibitory CD93 polypeptide is soluble. In some embodiments, the variant inhibitory CD93 polypeptide binds to IGFBP7 with a greater affinity than for MMNR2. In some embodiments, the variant inhibitory CD93 polypeptide binds to IGFBP7 with a greater affinity than CD93. In some embodiments, the inhibitory CD93 polypeptide comprises a F238 residue, wherein the amino acid numbering is based on SEQ ID NO: 1. In some embodiments, inhibitory CD93 polypeptide further comprises a stabilizing domain. In some embodiments, the stabilizing domain is an Fc domain. In some embodiments, the inhibitory polypeptide is about 50 to about 200 amino acids long.

In some embodiments, there is also provided a non-naturally variant inhibitory IGFBP7 polypeptide comprising a variant of IGFBP7, wherein the polypeptide blocks interaction between CD93 and IGFBP7. In some embodiments, the variant inhibitory IGFBP7 polypeptide binds to CD93 but does not activate CD93. In some embodiments, the variant inhibitory IGFBP7 polypeptide binds to CD93 with a greater affinity than for IGF-2, and or IGF1R. In some embodiments, the variant inhibitory IGFBP7 polypeptide hinds to CD93 with a greater affinity than IGFBP7. In some embodiments, the variant inhibitory IGFBP7 polypeptide comprises the IB domain of IGFBP7. In some embodiments, the variant inhibitory IGFBP7 polypeptide further comprises a stabilizing domain. In some embodiments, the stabilizing domain is an Fc domain. In some embodiments, the variant inhibitory is about 50 to about 200 amino acids long.

In some embodiments, there is also provided a pharmaceutical composition comprising the agent, the non-naturally occurring polypeptide, or the non-naturally occurring variant inhibitory IGFBP7 polypeptide as described above and a pharmaceutically acceptable carrier and/or excipient.

### BRIEF DESCRIPTION OF THE DRAWINGS

FIGS. 1A-1G show the identification of CD93 as a receptor protein on tumor vasculature regulated by VEGF signaling FIG. 1A shows a Venn Diagram depicting overlap of tumor vascular genes, which were significantly reduced by VEGF inhibitors from 4 different published RNA-Seq datasets (Log 2 fold change<−0.5). CD93 was the only gene found to be downregulated in all datasets, with 10 additional genes (listed, right) downregulated in 3 of 4 datasets. FIG. 1B depicts tube formation in HUVEC cells upon knocking down indicated gene respectively. FIG. 1C depicts an analysis of TCGA normal and GTEx datasets for CD93 transcription. FIG. 1D depicts representatives of IHC staining of human pancreas. PDA and PNET tumors for CD93 expression FIG. 1E depicts immunofluorescence (“IF”) staining of surface CD93 in mouse aortic endothelial cells (MAECs) cultured with or without VEGF. FIG. 1F depicts immunofluorescence staining of specimens from normal pancreas and tissues of orthotopic KPC tumor were stained for CD93 and CD31. FIG. 1G depicts immunofluorescence staining of specimens from normal skin and subcutaneously implanted B16 mouse tumors were stained for CD93 and CD31. Scale bar 50 μm.

FIGS. 2A-2E show that blocking the IGFBP7/CD93 interaction inhibits tumor growth and promotes vascular maturation. FIG. 2A depicts the change of tumor volume after treatment of control or mouse CD93 monoclonal antibody (“mAb”). B6 mice were challenged with KPC tumor cells and were started with the treatment of control or mouse CD93 mAb twice a week. Tumor growth was monitored over time n 10 mice/group. FIG. 2B depicts IF staining of CD31 in tumor sections from control and mCD93 mAb 7C10 treated mice. Blood vessel density, percentage of circular vessel and total vessel length were compared between groups. Arrows indicate circular blood vessels. Scale bar 50 μm. FIG. 2C depicts that frozen tumor sections were co-stained for CD31 and αSMA with quantification of percentage of αSMA vessel each field. Scale bar 50 μm FIG. 2D depicts that tumor sections were co-stained for CD31 and NG2 with quantification of percentage of NG2+ vessel each field. Each dot represents the mean value for one animal, of which at least five random fields were analyzed. Scale bar 50 μm. FIG. 2E depicts that KPC tumor-bearing mice were treated with control or CD93 mAb twice for a week and followed with assessment of tumor perfusion by intravenous lectin-FITC injection. Overlay of CD31 vessels with lectin-FITC delineates perfused and nonperfused tumor vessels. Quantification of perfused tumor vessels is presented on the right. Each dot represents the mean value for one animal with at least five random fields taken for each animal (n=5). * P<0.05, **P<0.01. p-value was determined by unpaired Student's t test. All data represent the mean±SEM.

FIGS. 3A-3F show that CD93 blockade promotes immune cell infiltration in tumors. FIG. 3A depicts representative images of CD3 and CD31 immunostaining and DAPI nuclear staining in implanted KPC tumors at day 8 and 15 after the starting treatment of control or anti-CD93. FIG. 3B depicts quantification of CD3− T cells in tumor tissues treated by control or anti-CD93. Each dot represents the mean value for one animal, with at least five random fields taken for each animal. FIGS. 3C-3E show flow cytometry analysis after 15 days antibody treatment. Flow cytometry analysis was performed to determine the percentages of CD45+ leukocytes infiltrating (FIG. 3C), the numbers of CD45− leukocytes, CD3− T cells, CD4+ and CD8+ cell subsets (FIG. 3D), and the percentages of granulocytic (CD3− CD11c− CD11b+ Ly6G+ Ly6C−) and monocytic (CD3− CD11c− CD11b− Ly6G− Ly6C+) MDSCs in CD45+ leukocytes (FIG. 3E) in the tumors. Each dot indicates one tumor. FIG. 3F shows representative images of CD3 and CD31 immunostaining in subcutaneous B16 mouse tumors 14 days after antibody treatment. Each dot represents the mean value for one animal, of which at least five random fields were analyzed. * P<0.05. ** P<=0.01. *** P<0.001. p-value was determined by unpaired Student's t test. All data represent the mean±SEM.

FIGS. 4A-4G show that IGFBP7 was identified as a binding partner for CD93. FIG. 4A depicts graphic views of subject wells with a positive hit (IGFBP7) for CD93-Ig in a human genome-scale receptor array (GSRA) screening system. The well containing an expression construct for Fc receptor (FcR) was used as a positive control FIG. 4B depicts HEK293T cells transduced with control or CD93 gene stained with IGFBP7-Ig for binding, with the presence of control, anti-CD93, or anti-IGFBP7 mAb as indicated. FIG. 4C depicts HUVEC cells stained with control or IGFBP7-Ig, with or without the presence of a mAb against hCD93. FIG. 4D HUVEC cell lysates were immunoprecipitated with control IgG or CD93 mAb, and blotted with CD93 and IGFBP7 antibodies. FIG. 4E depicts a microscale thermophoresis (MST) binding curve of human IGFBP7 to CD93. The Kd value is shown FIG. 4F depicts HEK293T cells transduced with control or mouse CD93 gene stained with mouse IGFBP7-Ig for binding. Monoclonal antibodies against mouse CD93 and IGFBP7 were added to evaluate their blocking capacities. FIG. 4G depicts schematic diagrams representing the structure of a series of chimeric proteins that were generated by replacing each domain of IGFBP7 (BP7) with a corresponding portion from IGFBP1(BPL1). The binding of each chimeric protein to CD93 transfectant was tested by flow cytometry. Binding index refers to mean fluorescence intensity (MFI) of CD93 transfectant divided by MFI of control.

FIGS. 5A-5E show the expression of IGFBP7 on tumor vascular endothelium FIG. 5A depicts H&E staining and IF co-staining of IGFBP7 and CD31 in human pancreas and PDA cancer. The percentages of IGFBP7-positive blood vessels in pancreatic ductal adenocarcinoma (PDAC) and normal pancreas were quantified. Each dot represents the mean value for one tissue, of which at least five random fields were analyzed. I: islet. Scale bar 50 μm. FIG. 5B depicts implanted KPC tumor tissue was co-stained for IGFBP7 and CD31, with the dash line separating central area (C) from the edge (E) of the tumor. Scale bar 100 μm. FIG. 5C depicts a representative western blot of HUVEC cells treated with DMOG (0, 10, and 24 hours) for HIF-1α and IGFBP7 expression. L: protein ladder. FIG. 5D shows IGFBP7 expression on mouse aortic endothelial cells (MAEC) detected by immunofluorescence. MAEC cells were incubated with dimethyloxaloylglycine (DMOG) to induce hypoxia, with or without a mouse VEGFR blocking mAb. The percentages of IGFBP7-expressing cells were quantified. Dots represent values from randomly taken fields. Scale bar 50 μm. FIG. 5E depicts a violin plot showing IGFBP7 expression in tumor endothelial cells from a xenograft colon cancer model (see Zhao Q., Cancer Research 2018:78(9):2370-82.) 24 hours after aflibercept treatment. * P<0.05. ***P<0.001. p-value was determined by unpaired Student's t test. All data represent the mean±SEM.

FIGS. 6A-6D show that targeting the IGFBP7/CD93 pathway improves drug delivery and facilitates chemotherapy, FIG. 6A shows immunofluorescence staining of doxorubicin and hypoxic (hypoxyprobe) in KPC tumor hearing mice treated with control or CD93 mAb. KPC tumorbearing mice treated with control or CD93 mAb twice for a week were injected with doxorubicin and pimonidazole for assessment of drug delivery and hypoxia, respectively. Penetration of doxorubicin and hypoxic (hypoxyprobe) areas within the tumor were quantified. Each dot represents one animal, of which the whole tumor tissue was analyzed. FIGS. 6B and 6C show tumor volume curves (FIG. 6B) and Kaplan-Meier survival analysis (FIG. 6C) of groups with the treatment of control, mCD93 mAb alone, 5-FU alone and the combination of mCD93 mAb and 5-FU, n=7. *P=0.045 **P=0.0163. B6 mice were subcutaneously implanted with 2×10⁵ B16 mouse melanoma cells, and were started with the treatment of antibody and 5-FU on day 6 when tumors became palpable. FIG. 6D shows immunofluorescence staining of Ki-67 and cleaved caspase 3 (CC3) in B16 mouse tumor tissues with the treatments of 5-FU alone and the combination of 5-FU and mCD93 mAb. The percentages of Ki-67-positive and CC3-positive cells in tumor tissues were quantified. Each dot represents one animal, of which the whole tumor tissue was analyzed Scale bar 50 μm. *P<0.05. **P<0.01. p-value was determined by unpaired Student's t test. All data represent the mean±SEM.

FIGS. 7A-7G show that CD93 blockage sensitizes tumors to anti-PD-1 therapy. FIG. 7A shows tumor weights after 14 days of antibody treatment. KPC tumor-hearing mice were started with the treatment of control or anti-CD93. In some groups CD4− or CD8− T cells were depleted by respective antibodies before anti-CD93 treatment. FIG. 7B depicts representative images of B7-H1 and CD31 immunostaining in subcutaneous KPC mouse tumors. FIG. 7C depicts flow cytometry analysis of single-cell suspensions of tumor tissues for B7-H1 expression. Percentages of B7-H1-positive cells in tumor cells, CD45− leukocytes, and CD31+ECs were determined. FIGS. 7D-7E show tumor growth curve (FIG. 7D) and tumor weight (FIG. 7E) 16 days post treatment with antibody as indicated in KPC tumorbearing mice. The treatment started 7 days after KPC tumor inoculation. FIGS. 7F-7G shows numbers of immune cells (FIG. 7F) and compositions (FIG. 7G) of immune cells within tumors determined by flow cytometry. (D-G) *P<0.05. **P<0.01. p-value was determined by unpaired Student's t test. All data represent the mean±SEM. Each dot represents one tumor (FIGS. 7A, 7C, and 7E-7G).

FIGS. 8A-8B show that anti-CD93 treatment does not affect proportions of T cell subsets within tumors. FIG. 8A depicts a FACS analysis of T cell subsets infiltrating the tumors upon 15 days of antibody treatment. FIG. 8B shows the analysis of intracellular cytokines IFN-γ and TNF-α in CD8+ T cell subset from freshly isolated tumor infiltrating lymphocytes (TILs) upon 4-hour PMA-Inomycin stimulation.

FIGS. 9A-9B show that anti-CD93 increases ICAM1 expression on tumor blood vessels. FIG. 9A shows representative images of ICAM-1 and CD31 immunostaining in tumor tissues from subcutaneous KPC mouse tumors after 14 days of antibody treatment. FIG. 9B shows representative images of CD45, CD31, and ICAM1 immunostaining in tumor tissues from subcutaneous B16 mouse tumors after 14 days antibody treatment.

FIGS. 10A-10B show identification of the binding domain on IGFBP7 for CD93. Each extracellular domain for IGFBP7, including insulin binding (IB). Kazal, and Ig, was swapped with the corresponding domain on IGFBPL1 using PCR cloning and fused to a C-terminal Ig. These chimeric mutants were transiently expressed in HEK293T cells and supernatants were used to stain CD93 transfectant. FIG. 10A depicts whether multiple chimeric IGFBP7 mutants bind to CD93. FIG. 10B depicts various human genes containing IB-domain constructed onto an expression vector containing Fc-Tag. Constructs were transiently transduced into HEK293T cells to produce Fc tagged fusion proteins in the supernatant. Supernatant was used to stain CD93 transfectant by flow cytometry. Binding index represents the ratio of binding MFI of CD93 transfectant to control cells.

FIGS. 11A-11B show IGFBP7 transcription in human PDA cancers. FIG. 11A depicts increased IGFBP7 transcript in human PDA than in normal pancreas. FIG. 11B depicts FACS analysis of TCGA PDA dataset indicating that transcription of IGFBP7 correlates with known endothelial cell markers, including PECAM1, CD34, VWF, and KDR (VEGFR2).

FIGS. 12A-12B show selective expression of IGFBP7 on mouse tumor vasculature. FIG. 11A depicts IF staining of IGFBP7 and CD31 in specimens from normal pancreas of naïve B6 mice and tissues from orthotopic KPC mouse tumor. I refers to islet. FIG. 11B depicts IF staining of IGFBP7 and CD31 in specimens from normal skin of naïve B6 mice and tissues from subcutaneously implanted KPC and B16 mouse tumors. Scale bar 50 μm.

FIGS. 13A-13C show that blocking the IGFBP7/CD93 interaction inhibits vascular angiogenesis and tumor growth. FIG. 13A depicts results of a tube formation assay performed in IGFBP7 knockdown and control HUVEC cells. FIGS. 13B-13C depict results of a tube formation assay (FIG. 13B) and transwell migration assay (FIG. 13C) performed with or without exogenous IGFBP7 protein in WT or CD93 knockdown HUVEC cells.

FIGS. 14A-14F show that IGFBP7 blockade retards tumor growth and promotes tumor vascular maturation. FIG. 14A shows that mouse IGFBP7 bind to MAEC cells, and the interaction can be blocked by an IGFBP7 mAb (clone 2C6). FIG. 14B shows tumor volume change after treatment of an IGFBP7 antibody. C57BL/6 mice with palpable KPC tumors were treated with control or mIGFBP7 mAb (Clone 2C6) twice a week. Tumor growth was monitored over time (n 10 mice/group). FIG. 14C depicts IF staining of CD31 on frozen tumor sections. Blood vessel density, percentage of circular vessel and total vessel length were compared between groups. Arrows indicate circular blood vessels. Scale bar 50 μm. FIGS. 14D-14E depicts representative images of IF staining of CD31 and αSMA (FIG. 14D), or CD31 and NG2 (FIG. 14E) on frozen KPC mouse tumor sections. Each dot represents a random field from three animals, with at least three random fields taken from each animal. FIG. 14F depicts representative images of IF staining of CD31 and activated integrin β1 (9EG7) with quantification of 9EG7 vessel (% of total vessels) on KPC mouse tumor sections. Each dot represents the mean value for one animal, with at least five random fields taken for each animal. Scale bar 50 μm.

FIG. 15 shows that human IGFBP7 fails to bind human IGF1R transfectant. Wild type CHO and IGF1R transfected CHO cells were stained for human IGF1R staining antibody to confirm surface IGF1R expression. At the same time, cells were incubated with IGFBP7-Ig for possible interaction by flow cytometry analysis,

FIG. 16 shows the capacity of various commercial anti-human IGFBP7 mAbs and anti-CD93 mAb for blocking CD93/IGFBP7 interaction.

FIGS. 17A-17B show that CD93 on nonhematopoietic cells mediates antitumor effect by blocking CD93. FIG. 17A depicts representative images of IF staining of B16 tumors detected injected anti-CD93 on tumor vasculature (CD31+). FIG. 17B shows tumor growth in CD93 chimeric mice after anti-CD93 antibody treatment. WT B6 mice reconstituted with hone marrow (BM) cells from WT or CD93KO mice were inoculated with B16 tumor cells and followed with antibody treatment. ***p<0.001.

FIGS. 18A-18C show that CD93 blockade inhibits mouse tumor growth. Both CD93 (FIG. 18A) and IGFBP7 (FIG. 18B) were upregulated in tumor vasculature of subcutaneous B16 tumors. FIG. 18C shows tumor growth after anti-CD93 antibody treatment. Mice with palpable B16 tumors received treatment with control or anti-CD93 (7C10), n=10. **p<0.01.

FIGS. 19A-19G show that CD93 blockade promotes a favorable tumor immune microenvironment. FIG. 19A depicts representative images of CD3 and CD3 immunostaining of B16 tumors two weeks after antibody treatment. FIGS. 19B and 19B show flow cytometry analysis of infiltrating CD45− leukocytes (FIG. 10B) and immune cell subsets (FIG. 10C) in B16 tumor. Anti-CD93 increased the percentages of T_{EM} (CD44hi CD62L−). PD1- and Granzyme B1 cells (FIG. 19D), as well as cytokine producing cells in CD8+ TILs (FIG. 19E), FIG. 19F shows the effect of anti-CD93 treatment on PD1-cells. T_{EM} cells and Treg cells. The same treatment caused an increase of PDL1 and T_{EM} cells, accompanied with a reduction of Treg cells in CD4− T cell compartment. FIG. 19G shows representative images of IF staining B16 tumor tissues. IF staining resealed a reduction of hypoxic area and less CD11b− suppressors in anti-CD93 treated tumors. *p<0.05, **p<0.01, *** p<0.001.

FIGS. 20A-20E show that CD93 blockade sensitizes B16 melanoma to immune checkpoint blockade (ICB) therapy. FIG. 20A shows representative images of B16 tumors under antibody treatment stained for PD-L1, CD31, and CD45. FIG. 20B shows flow cytometry analysis of PD-L1 on different cell hypes. B6 mice with palpable B16 tumors were treated with indicated antibodies twice/week. FIG. 20C depicts tumor growth and survival curves. FIG. 20D shows quantification of intratumoral immune cells. FIG. 20E shows quantification of T_{EM} cells (CD44ₕᵢ CD62L−) in different T cell subsets. *p<0.05, **p<0.01, ***p<0.001.

FIGS. 21A-21D show that the IGFBP7/CD93 pathway is upregulated in triple-negative breast cancer (TNBC) vasculature. Representative images of IF staining of CD93 (FIGS. 21A, 21C) and IGFBP7 (FIGS. 21B, 21D) in human TNBC (FIGS. 21A, 21B) and mouse 4T1 (FIGS. 21C, 21D) tumors are shown. CD93 is used for staining blood vessels.

FIG. 22 shows that IGFBP7 expression is associated with poor prognosis in TNBC.

FIGS. 23A-23B show that anti-CD93 inhibits orthotopic BC tumor growth in vivo. Mice were orthotopically implanted with 4T1 (FIG. 23A) or PY8119 (FIG. 23B). When palpable, mice were treated with control or anti-CD93 mAb (clone 7C10, 10 mg/kg) twice a week.

FIGS. 24A-24C show that blockade of CD93 signaling promotes tumor vascular maturation in orthotopic 4T1. Ten days post anti-CD93 treatment. 4T1 tumor tissues were stained for αSMA (FIG. 24A) and NG2 (FIG. 24B) to examine pericyte coverage on CD31-vessels. Blood vessels were enumerated by CD31 staining. FIG. 24C shows that anti-CD93 treatment significantly reduces tumor hypoxia and increases perfusion, revealed by pimonidazole and Lectin-FITC staining, respectively

FIGS. 25A-25C show that CD93 blockade promotes a favorable TME. 4T1 tumors under the treatment of anti-CD93 displayed more CD3+ T cell infiltrates, accompanied with less intratumoral MDSCs, based on IF (FIGS. 25A, 25B) and FACS analysis (FIG. 25C).

FIGS. 26A-26C show that IGFBP7 and CD93 are upregulated in vasculatures within human cancers. IGFBP7 (FIG. 26A) and CD93 (FIG. 26B) are upregulated in vasculatures within human cancers, including kidney, head and neck, as well as colon. FIG. 26C shows that both CD93 and IGFBP7 are upregulated in melanoma-associated endothelium.

FIGS. 27A-27B show enrichment of the IGFBP7/CD93 pathway in human cancers resistant to anti-PD therapy. FIG. 27A shows expression levels of IGFBP7 and CD93 in patients with metastatic urothelial cancer. In a phase II trial of patients with metastatic urothelial cancer treated with anti-PD-L1 (77), the expression levels of IGFBP7 and CD93 were compared between non-responders (SD/PD) and responders (CR/PR). Statistical analysis was performed using Wilcoxon rank sum test. FIG. 27B shows expression levels of IGFBP7 and CD93 in melanoma patients. In a cohort of melanoma patients under anti-PD-1 therapy (78), the expressions of IGFBP7 and CD93 in responders and non-responders were determined. Statistical analysis was performed using unpaired Student's t test.

FIGS. 28A-28E demonstrate that IGFBP7 and MMRN2 bind to different motifs on CD93. In FIG. 28A, binding of HEK293 T cells transfected to express group 14 C-type lectin molecules were stained for the binding of IGFBP7-Ig and MMRN2-Ig. In FIG. 28B. CHO cells stably expressing CD93 were stained with control or MMRN2-Ig, with or without the presence of IGFBP7-His protein. In FIG. 28C, well coated with IGFBP7-His protein were incubated with CD93-His protein before examining for MMRN2-Ig binding by ELISA. Wells coated with CD93-His protein were used as a positive control. In FIG. 28D. HEK293T cells transfected with control or CD93 construct were stained with MMRN-Ig for binding, with or without the presence of anti-mCD93 (7C10). In FIG. 28E HEK293T cells transfected to express different mouse CD93 mutants were stained with anti-CD93 (7C10), IGFBP7-Ig, and MMRN2-Ig.

### DETAILED DESCRIPTION OF THE APPLICATION

The present application provides methods and compositions useful for promoting a favorable tumor microenvironment for therapeutic interventions. The leaky and irregular vascular network within solid tumor poses a great obstacle to drug delivery and impairs immune cell infiltration. It was a novel discovery by the inventors of this application that insulin growth factor binding protein 7 (IGFBP7) transmits a signal via CD93 that is pivotal for this abnormality. The expression of CD93 and IGFBP7, controlled by VEGF signaling, are both upregulated in tumor tissues. It was surprisingly found that disruption of the IGFBP7 and CD93 interaction by either IGFBP7 or CD93 monoclonal antibodies attenuates tumor growth and promotes vascular maturation. CD93 blockade increases tumor perfusion, reduces hypoxia and facilitates chemotherapy. Moreover, targeting CD93 promotes intratumoral T cell infiltration and thereby sensitizes tumors to anti-PD1 antibody therapy. The present application, thus, identifies a novel molecular interaction that is responsible for abnormal tumor vascularization and offers novel approaches to cancer therapy.

The present application provides agents that specifically inhibit the IGFBP7/CD93 signaling pathway, such as agents that specifically block the interaction between CD93 and IGFBP7. Suitable agents include blocking antibodies specifically recognizing CD93, blocking antibodies specifically recognizing IGFBP7, inhibitory CD93 polypeptides comprising at least a portion of the extracellular domain of CD93 or variant thereof, inhibitory polypeptides comprising a variant of IGFBP7, and other agents such as peptides, peptide analogs, fusion peptides, aptamers, an avimer, an anticalin, a speigelmer, small molecule compounds, siRNAs, shRNAs, miRNAs, antisense RNAs, and gene editing systems. These agents are useful for treating cancer or contributing to one or more aspects of cancer treatment such as blocking abnormal tumor vascular angiogenesis, normalizing immature and leaky blood vessels, promoting formation of functional vascular network in tumors, promoting vascular maturation, promoting favorable tumor microenvironment, increasing immune cell infiltration in tumors, increasing tumor perfusion, and reducing hypoxia in tumors. The agents described herein are also useful for sensitizing a tumor to a second therapy or facilitating delivery of a second therapeutic agent. The agents described herein thus are particularly useful for combination therapy, for example combination with chemotherapeutic agent and immunomodulating agents.

Thus, in one aspect, there is provided a method of treating cancer or one or more aspects of cancer treatment in a subject, comprising administering to the subject an effective amount of an agent that specifically inhibits the IGFBP7/CD93 signaling pathway (such as an agent that specifically blocks the interaction between CD93 and IGFBP7).

In another aspect, there are provided novel agents (such as anti-CD93, anti-IGFBP7, inhibitory CD93 polypeptides, and inhibitory IGFBP7 polypeptides) that specifically block the interaction between CD93 and IGFBP7.

In another aspect, there are provided methods of identifying agents that are useful for cancer treatment (such as agents that specifically block the interaction between CD93 and IGFBP7), for example in a high throughput screening context.

Also provided are kits, agents (such as any of the agents described herein), polynucleotides encoding the agents (such as any of the agents described herein), and reagents (such as an isolated CD93/IGFBP7 complex) useful for the methods described herein.

### I. Definitions

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

It is understood that embodiments of the application described terms of “comprising” herein include “consisting” and/or “consisting essentially of” embodiments.

An agent that inhibits the interaction between CD93 and IGFBP7 refers to any agent that reduces the level of binding between CD93 and IGFBP7, as compared to the level of binding between CD93 and IGFBP7 in the absence of the agent. In some embodiments, the agent is one that reduces the level of binding between CD93 and IGFBP7 by at least about 10%, 20%, 30%, 40% or 50%, 60%, 70%, 80%, 90%, 95% or 99%, In some embodiments, the agent is one that reduces the level of binding between CD93 and IGFBP7 to an undetectable level, or eliminates binding between CD93 and IGFBP7. Suitable methods for detecting and/or measuring (quantifying) the binding of CD93 to IGFBP7 are well known to those skilled in the art, and include those described herein.

“Angiogenesis” refers to the process by which new blood vessels sprout from existing vessels.

The term “antibody” is used in its broadest sense and encompasses various antibody structures, including but not limited to monoclonal antibodies, polyclonal antibodies, multispecific antibodies (e.g., bispecific antibodies), humanized antibodies, chimeric antibodies, full-length antibodies and antigen-binding fragments thereof, so long as they exhibit the desired antigen-binding activity. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv.” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994), incorporated herein by reference in its entirety for all purposes.

“Diabody” or “diabodies” described herein refer to a complex comprising two scFv polypeptides. In some embodiments, inter-chain but not intra-chain pairing of the VH and VL domains is achieved, resulting in a bivalent fragment, i.e., fragment having two antigen-binding sites.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta. Curr. Op. Struct. Biol. 2:593-596 (1992), each of which are incorporated herein by reference in their entirety for all purposes.

As used herein, a first antibody “competes” for binding to a target (e.g., CD93 or IGFBP7) with a second antibody when the first antibody inhibits target binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731 incorporated herein by reference in its entirety for all purposes.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE, software. Those skilled in the art can determine appropriate parameters for measuring alignment including any algorithms needed to achieve maximal alignment over the full-length of the sequences being compared. For purposes herein, however, % amino acid sequence identity values are generated using the sequence comparison computer program MUSCLE (Edgar. R. C., Nucleic Acids Research 32(5):1792-1707, 2004; Edgar, R. C., BMC Bioinformatics 5(1): 113, 2004, each of which are incorporated herein by reference in their entirety for all purposes).

“Homologous” refers to the sequence similarity or sequence identity between two polypeptides or between two nucleic acid molecules. When a position in both of the two compared sequences is occupied by the same base or amino acid monomer subunit. e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “epitope” as used herein refers to the specific group of atoms or amino acids on an antigen to which an antibody or diabody binds. Two antibodies or antibody moieties may bind the same epitope within an antigen if they exhibit competitive binding for the antigen.

As used herein, a first antibody (such as a diabody) “competes” for binding to a target antigen with a second antibody (such as a diabody) when the first antibody inhibits the target antigen binding of the second antibody by at least about 50% (such as at least about any one of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731 incorporated herein by reference in its entirety for all purposes.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation. ADP-ribosylation, pegylation, biotinylation, etc.).

As use herein, the terms “specifically binds,” “specifically recognizing,” and “is specific for” refer to measurable and reproducible interactions, such as binding between a target and an antibody (such as a diabody). In certain embodiments, specific binding is determinative of the presence of the target in the presence of a heterogeneous population of molecules including biological molecules (e.g., cell surface receptors), for example, an antibody that specifically recognizes a target (which can be an epitope) is an antibody (such as a diabody) that binds this target with greater affinity, avidity, more readily, and/or with greater duration than its bindings to other molecules. In some embodiments, the extent of binding of an antibody to an unrelated molecule is less than about 10% of the binding of the antibody to the target as measured, e.g., by a radioimmunoassay (RIA). In some embodiments, an antibody that specifically binds a target has a dissociation constant (KD) of <10⁻⁵ M, 10⁻⁶ M, <10⁻⁷ M, <10⁻⁸ M, <10⁻⁹ M, <10⁻¹⁰M, <10⁻¹¹ M, or <10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA, RIA, ECL, IRMA, EIA, BIACORE™ and peptide scans.

As used herein, “the composition” or “compositions” includes and is applicable to compositions of the application. The application also provides pharmaceutical compositions comprising the components described herein.

As used herein, “treatment” or “treating” is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., presenting or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and, or prolonging survival. Also encompassed by “treatment” is a reduction of a pathological consequence of a hyperplasia, such as tumor (e.g., cancer), restenosis, or pulmonary hypertension. The methods of the application contemplate any one or more of these aspects of treatment. The benefit to a subject to be treated is either statistically significant or at least perceptible to the patient or to the physician.

The term “effective amount” used herein refers to an amount of an agent or composition sufficient to treat a specified state, disorder, condition, or disease such as ameliorate, palliate, lessen, and/or delay one or more of its symptoms (e.g., clinical or sub-clinical symptoms). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. In reference to a hyperplasia (e.g. cancer, restenosis, or pulmonary hypertension), an effective amount comprises an amount sufficient to cause a hyperplastic tissue (such as a tumor) to shrink and/or to decrease the growth rate of the hyperplastic tissue (such as to suppress hyperplastic or tumor growth) or to prevent or delay other unwanted cell proliferation in the hyperplasia. In some embodiments, an effective amount is an amount sufficient to delay development of a hyperplasia (e.g. cancer, restenosis, or pulmonary hypertension). In some embodiments, an effective amount is an amount sufficient to prevent or delay recurrence. An effective amount can be administered in one or more administrations. In the case of cancer, the effective amount of the drug or composition may: (i) reduce the number of tumor cells; (ii) reduce the tumor size; (iii) inhibit, retard, slow to some extent and preferably stop a tumor cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, incorporated by reference in its entirely for all purposes.

The term ‘tumor’ refers to or describes the physiological condition in mammals that is typically characterized by unregulated cell growth and includes benign or malignant abnormal growth of tissue The term “tumor” includes cancer.

The terms “subject,” “individual,” and “patient” are used interchangeably herein to refer to a mammal, including, but not limited to, human, bovine, horse, feline, canine, rodent, or primate. In some embodiments, the subject is a human. In a preferred embodiment, the subject is a human.

Reference to “about” a value or parameter herein includes (and describes) variations that are directed to that value or parameter per se. For example, description referring to “about X” includes description of “X”. In certain embodiments, a range can be within an order of magnitude, preferably within 50%, more preferably within 20%, still more preferable within 10%, and even more preferably within 5% of a given value or range. The allowable variation encompassed by the term “about” or “approximately” depends on the particular system under study, and can be readily appreciated by one of ordinary skill in the art.

The term “about X-Y” used herein has the same meaning as “about X to about Y.”

As used herein and in the appended claims, the singular forms “a,” “an,” “or,” and “the” include plural referents unless the context clearly dictates otherwise. Thus, for example, a reference to “a method” includes one or more methods, and/or steps of the type described herein and/or which will become apparent to those persons skilled in the art upon reading this disclosure. As is apparent to one skilled in the art, a subject assessed, selected for, and/or receiving treatment is a subject in need of such activities.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual, 3rd ed. Cold Spring Harbor Laboratory Press: Cold Spring Harbor, N.Y.; Ausubel et al. eds. (2005) Current Protocols in Molecular Biology. John Wiley and Sons, Inc.; Hoboken, N.J.; Bonifacino et al. eds. (2005) Current Protocols in Cell Biology. John Wiley and Sons, Inc.; Hoboken, N.J.; Coligan et al. eds. (2005) Current Protocols in Immunology, John Wiley and Sons, Inc.; Hoboken, N.J.; Coico et al. eds. (2005) Current Protocols in Microbiology, John Wiley and Sons, Inc.; Hoboken, N.J.; Coligan et al. eds. (2005) Current Protocols in Protein Science, John Wiley and Sons. Inc.; Hoboken, N.J.; and Enna et al. eds. (2005) Current Protocols in Pharmacology, John Wiley and Sons, Inc.; Hoboken, N.J. Additional techniques are explained e.g., in U.S. Pat. No. 7,912,698 and U.S. Patent Appl. Pub. Nos. 2011/0202322 and 2011/0307437, each of which is incorporated by reference in their entirety for all purposes.

The terms and expressions which have been employed are used as terms of description and not of limitation, and use of such terms and expressions do not exclude any equivalents of the features shown and described or portions thereof, and various modifications are possible within the scope of the technology claimed.

### II. Methods of Treatment

The present application in one aspect provides a method of treating tumor (such as cancer) or one or more aspects of tumor (such as cancer) treatment in a subject, comprising administering to the subject an effective amount of an agent that specifically inhibits the IGFBP7/CD93 signaling pathway (such as an agent that blocks the interaction between CD93 and IGFBP7). An agent “blocks the interaction between CD93 and IGFBP7” if the agent reduces binding between CD93 and IGFBP7 as compared to the level of binding between CD93 and IGFBP7 in the absence of the agent. In some embodiments, the agent reduces the binding of CD93 and IGFBP7 by at least about 10%, 20%, 30%, 40%, or 50%. In some embodiments, the agent reduces the binding of CD93 and IGFBP7 by at least about 60%, 70%, 80%, 90%, or more. In some embodiments, the agent blocks the CD93/IGFBP7 interaction to an undetectable level, or eliminates the binding between CD93 and IGFBP7.

Suitable methods for determining the binding of CD93 and IGFBP7 are known in the art, and can include for example ELISA, pull-down assays, surface plasmon resonance assays, chip-based assays, FACS, yeast two-hybrid systems, phage display, and FRET.

The agents described herein can be administered directly, or may be administered in the form of a polynucleotide encoding the agent. Thus, as used herein, the term “administering to the subject” encompasses both administering the agent directly to the subject and administering a polynucleotide that encodes the agent, for example via a vector.

In some embodiments, there is provided a method of treating a tumor (such as a cancer) in a subject, comprising administering to the subject an effective amount of an agent that specifically inhibits the IGFBP7/CD93 signaling pathway (such as an agent that blocks the interaction between CD93 and IGFBP7). In some embodiments, the agent is an antibody, a peptide, a polypeptide, a peptide analog, a fusion peptide an aptamer, an avimer, an anticalin, a speigelmer, a small molecule compound, a siRNA, a shRNA, a miRNAs, an antisense RNA, or a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of blocking abnormal tumor vascular angiogenesis in a subject, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of normalizing immature and leaky blood vessel in a subject, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of promoting formation of functional vascular network in a tumor in a subject, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of promoting vascular maturation in a tumor in a subject, comprising administering to the subject an effective amount of an agent that specifically inhibits the IGFBP7/CD93 signaling pathway (such as an agent that blocks the interaction between CD93 and IGFBP7). In some embodiments, there is provided a method of promoting vascular normalization in a tumor in a subject, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell). In some embodiments, vascular normalization is characterized by increased association of pericytes and/or smooth muscle cells with the endothelial cells lining the walls of the vessels, formation of a more normal basement membrane (e.g., having a more physiological thickness) and/or closer association of vessels with the basement membrane. In some embodiments, the normalization of vascular described herein does not involve a decreased number of vessels (e.g., a less dense network).

In some embodiments, there is provided a method of promoting favorable tumor microenvironment in a subject, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of increasing immune cell infiltration in a tumor in a subject, comprising administering to the subject an effective amount of an agent that specifically inhibits the IGFBP7/CD93 signaling pathway (such as an agent that blocks the interaction between CD93 and IGFBP7). In some embodiments, the method increases infiltration of CD3− cells (such as tumor infiltrating leukocytes (“TILs”)). In some embodiments, the method increases infiltration of CD45− cells (such as TILs). In some embodiments, the method increases infiltration of CD8− cells (such as NK cells or T cells) In some embodiments, the method increases the immune cell infiltration into a tumor by at least about any of 20%, 30%, 40%, 50%, or more. In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IFGBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of increasing tumor perfusion in a subject, comprising administering to the subject an effective amount of an agent that specifically inhibits the IGFBP7/CD93 signaling pathway (such as an agent that blocks the interaction between CD93 and IGFBP7). In some embodiments, the tumor perfusion is increased by at least about any of 20%, 30%, 40%, 50%, or more. In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic, agent (such as a chemotherapeutic agent, an immunomodulatory, or an immune cell).

In some embodiments, there is provided a method of reducing hypoxia in tumor in a subject, comprising administering to the subject an effective amount of an agent that specifically inhibits the IGFBP7/CD93 signaling pathway (such as an agent that blocks the interaction between CD93 and IGFBP7). In some embodiments, the tumor hypoxia is reduced by at least about any of 20%, 30%, 40%, 50%, or more. In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of reducing immunosuppressive cells (such as Treg cells, granulocytic myeloid-derived suppressor cells (gMDSC), and tumor-associated macrophages (Mac)) in a subject, comprising administering to the subject an effective amount of an agent that specifically inhibits the IGFBP7/CD93 signaling pathway (such as an agent that blocks the interaction between CD93 and IGFBP7). In some embodiments, the method reduces immunosuppressive cells in the tumor microenvironment. In some embodiments, the immunosuppressive cells are reduced by at least about any of 20%, 30%, 40%, 50%, or more. In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognising CD93. In some embodiments, the agent is a blocking antibody specifically recognising IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject a second therapeutic agent (such as a chemotherapeutic agent, an immunomodulatory, or an immune cell).

In some embodiments, there is provided a method of sensitising a tumor to a second therapy, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IFGBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises subjecting the subject to the second therapy (such as chemotherapy, immunotherapy, cell therapy, radiation therapy, etc.). In some embodiments, the second therapy is immunotherapy. In some embodiments, the second therapy comprises administration of an immune checkpoint inhibitor, including for example an anti-PD1 antibody, an anti-PD-L1 antibody, an anti-CTLA4 antibody, or a combination thereof such as an anti-PD1 antibody and an anti-CTLA4 antibody.

In some embodiments, there is provided a method of facilitating delivery of a second therapeutic agent (such as a chemotherapeutic agent or an immunomodulating agent), comprising administering to the subject an effective amount of an agent that specifically inhibits the IGFBP7/CD93 signaling pathway (such as an agent that blocks the interaction between CD93 and IGFBP7). In some embodiments, there is provided a method of improving the efficacy of a second therapeutic agent (such as a chemotherapeutic agent or an immunomodulating agent), comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer, an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject the second therapeutic agent (such as a chemotherapeutic agent, an immunomodulatory, or an immune cell) sequentially, simultaneously, and/or concurrently. In some embodiments, the second therapeutic agent is an immune checkpoint inhibitory including for example an anti-PD1 antibody, an anti-PD-L1 antibody, an anti-CTLA4 antibody, or a combination thereof such as an anti-PD1 antibody and an anti-CTLA4 antibody.

The agents described herein are also useful for one or more of the following: 1) increasing pericyte-covered blood vessel: 2) increasing vascular length of blood vessel with circular shape: 3) increasing alpha smooth muscle actin (α-SMA)-positive cells associated with blood vessels, and 4) reducing β1 integrin activation. In some embodiments, there is provided a method of increasing pericyte-covered blood vessel, comprising administering to the subject an effective amount of an agent that specifically inhibits the IGFBP7/CD93 signaling pathway (such as an agent that blocks the interaction between CD93 and IGFBP7). In some embodiments, there is provided a method of increasing vascular length of blood vessel with circular shape, comprising administering to the subject an effective amount of an agent that specifically inhibits the IGFBP7/CD93 signaling pathway (such as an agent that blocks the interaction between CD93 and IGFBP7). In some embodiments, there is provided a method of increasing alpha smooth muscle actin (α-SMA)-positive cells associated with blood vessels, comprising administering to the subject an effective amount of an agent that specifically inhibits the IGFBP7/CD93 signaling pathway (such as an agent that blocks the interaction between CD93 and IGFBP7). In some embodiments, there is provided a method of reducing β1 integrin activation, comprising administering to the subject an effective amount of an agent that specifically inhibits the IGFBP7/CD93 signaling pathway (such as an agent that blocks the interaction between CD93 and IGFBP7). In some embodiments, the agent is selected from the group consisting of an antibody, a peptide, a polypeptide, a peptide analog, a fusion peptide, an aptamer, an avimer an anticalin, a speigelmer, a small molecule compound, a siRNA, a shRNA, a miRNAs, an antisense RNA, and a gene editing system. In some embodiments, the agent is a blocking antibody specifically recognizing CD93. In some embodiments, the agent is a blocking antibody specifically recognizing IGFBP7. In some embodiments, the agent is an inhibitory CD93 polypeptide comprising at least a portion of the extracellular domain of CD93 or variant thereof. In some embodiments, the agent is an inhibitory polypeptide comprising a variant of IGFBP7. In some embodiments, the method further comprises administering to the subject the second therapeutic agent (such as a chemotherapeutic agent, an immunomodulatory, or an immune cell) sequentially, simultaneously, and/or concurrently.

In some embodiments, the methods described herein comprise administering to the subject an effective amount of an anti-CD93 antibody that specifically recognizes CD93 and blocks interaction between CD93 and IGFBP7. In some embodiments, the anti-CD93 antibody further blocks interaction between CD93 and MMNR2. In some embodiments, the anti-CD93 antibody does not block the interaction between CD93 and MMNR2. In some embodiments, the anti-CD93 antibody binds to the IGFBP7 binding site on CD93. In some embodiments, the anti-CD93 antibody binds to a region of CD93 that is outside of the IGFBP7 binding site, for example, a site that is required for a stable interaction and thus the binding indirectly affects binding to IGFBP7. In some embodiments, the anti-CD93 antibody hinds to CD93 competitively against mAb MM01 or mAb 7C10. In some embodiments, the anti-CD93 antibody binds to an epitope that overlaps or substantially overlap with that of mAb MM01 or mAb 7C10. In some embodiments, the anti-CD93 antibody binds to an epitope that does not substantially overlap with that of mAb MM01 or mAb 7C10. In some embodiments, “substantially overlap” described above refers to the scenario that at least about 50%, 60%, 70%, 80%, or 90% of the residues on CD93 that the anti-CD93 antibody binds to overlap with the residues that mAb MM01 or mAb 7C10 binds to. In some embodiments, the anti-CD93 antibody is mAb MM01 or a humanised version thereof. In some embodiments, the method further comprises administering to the subject a second therapeutic agent (such as a chemotherapeutic agent, an immunomodulatory, or an immune cell). In some embodiments, the second therapeutic agent is an immune checkpoint inhibitory (such as an anti-PD1 antibody, an anti-PD-L1 antibody, an anti-CTLA4 antibody, or a combination thereof such as the combination of an anti-PD1 antibody and an anti-CTLA4 antibody).

In some embodiments, the methods described herein comprise administering to the subject an effective amount of a polypeptide comprising at least a portion of the extracellular domain of CD93 or a variant thereof that specifically blocks interaction between CD93 and IGFBP7 (inhibitory CD93 polypeptide). In some embodiments, the method further comprises administering to the subject a second therapeutic agent (such as a chemotherapeutic agent, an immunomodulatory, or an immune cell). In some embodiments, the second therapeutic agent is an immune checkpoint inhibitory (such as an anti-PD1 antibody, an anti-PD-L1 antibody, an anti-CTLA-4 antibody, or a combination therefore, such as a combination of an anti-PD-L1 antibody and an anti-CTLA4 antibody). In some embodiments, the inhibitory CD93 polypeptide further comprises a stabilising domain (such as Fc). In some embodiments, the inhibitory CD93 polypeptide is about 50 to about 100 amino acids long. In some embodiments, the CD93 portion of the inhibitory CD93 polypeptide, i.e., the portion that corresponds to the extracellular domain of CD93 or a portion thereof and conveys the function of blocking binding of CD93 and IGFBP7, is about 50 to about 100 amino acids long. In some embodiments, the inhibitory CD93 polypeptide comprises an F238 residue, wherein the amino acid numbering is based on SEQ ID NO: 1.

In some embodiments, the inhibitory CD93 polypeptide is a soluble polypeptide. In some embodiments, the inhibitory CD93 polypeptide is membrane bound, for example via a GPI linkage. In certain embodiments, the membrane bound inhibitory CD93 polypeptide is cleaved from the membrane prior to administration. These inhibitory CD93 polypeptides can be administered to a subject via any administration routes such as intravenous route. Alternatively, the inhibitory polypeptide can be administered to the subject via administration of a polynucleotide encoding the inhibitory CD93 polypeptide, e.g., via a vector platform.

In some embodiments, the inhibitory CD93 polypeptide is bound to the membrane via a transmembrane domain. Such inhibitory CD93 polypeptide can be introduced into the subject by introducing a polynucleotide (such as cDNA or mRNA) encoding the inhibitory polypeptide into a cell in the subject and causing expression of the inhibitory CD93 polypeptide on the cell surface. For example, the membrane bound inhibitory CD93 polypeptide can be a dominant-negative form of CD93 that binds to IGFBP7 but is unable to transmit a signal downstream. The dominant-negative form of CD93 may comprise one or more mutation that inactivates the intracellular signaling domain of CD93. Alternatively, the dominant-negative form of CD93 lacks the intracellular domain of CD93.

Also contemplated herein are inhibitory CD93 polypeptides that comprise one or more mutations in the extracellular domain, such as mutations that allows the inhibitory CD93 polypeptide to show preferential binding to IGFBP7 over other binding partners of CD93 such as MMNR2. In some embodiments, the inhibitory CD93 polypeptide binds to IGFBP7 with a greater affinity than to MMNR2. In some embodiments, the inhibitory CD93 polypeptide binds to IGFBP7 with a greater affinity as compared to wild tape CD93.

In some embodiments, the methods described herein comprise administering to the subject an effective amount of an anti-IGFBP7 antibody that specifically recognises IGFBP7 and blocks interaction between CD93 and IGFBP7. In some embodiments, the anti-IGFBP7 antibody further blocks interaction between IGFBP7 and one or more of its other binding partners, such as IGF-1, IGF-2, and IGF1R. In some embodiments, the anti-IGFBP7 antibody does not block the interaction between IGFBP7 and one or more of its binding partners. In some embodiments, the anti-IGFBP7 antibody binds to the CD93 binding site on IGFBP7. In some embodiments, the anti-IGFBP7 antibody binds to a region of IGFBP7 that is outside of the CD93 binding site, for example a site that is required for a stable interaction and thus the binding indirectly affects binding to CD93. In some embodiments, the anti-IGFBP7 antibody binds to the insulin binding (IB) domain of IGFBP7. In some embodiments, the anti-IGFBP7 antibody binds to IGFBP7 competitively against mAb R003 or mAb 2C6. In some embodiments, the anti-IGFBP7 antibody binds to an epitope that overlaps or substantially overlap with that of mAb R003 or mAb 2C6. In some embodiments. “substantially overlap” described above refers to the scenario that at least about 50%, 60%, 70% 80%, or 90% of the residues on IGFBP7 that the anti-IGFBP7 antibody binds to overlap with the residues that mAb R003 or mAb 2C6 binds to. In some embodiments, the anti-IGFBP7 antibody is mAb R003 or a humanized version thereof. In some embodiments, the method further comprises administering to the subject a second therapeutic agent (such as a chemotherapeutic agent, an immunomodulatory, or an immune cell). In some embodiments, the second therapeutic agent is an immune checkpoint inhibitor (such as an anti-PD1 antibody or an anti-PD-L1 antibody).

In some embodiments, the methods described herein comprise administering to the subject an effective amount of a polypeptide comprising a variant of IGFBP7 that specifically blocks interaction between CD93 and IGFBP7 (inhibitory IGFBP7 polypeptide), which includes but is not limited to, a mutant form of IGFBP7 and a fragment (portion) of IGFBP7. In some embodiments, the method further comprises administering to the subject a second therapeutic agent (such as a chemotherapeutic agent, an immunomodulatory, or an immune cell). In some embodiments, the second therapeutic agent is an immune checkpoint inhibitor (such as an anti-PD1 antibody or an anti-PD-L1 antibody). In some embodiments, the inhibitory IGFBP7 polypeptide further comprises a stabilizing domain (such as Fc). In some embodiments, the inhibitory IGFBP polypeptide is about 50 to about 100 amino acids long.

In some embodiments, the IGFBP portion of the inhibitory IGFBP7 polypeptide, i.e., the portion that corresponds to IGFBP7 or a portion thereof and conveys the function of blocking binding of CD93 and IGFBP7, is about 50 to about 100 amino acids long. In some embodiments, the inhibitory IGFBP7 polypeptide comprises the IB domain of IGFBP7. In some embodiments, the inhibitory IGFBP7 polypeptide does not comprises am domains of IGFBP7 other than the IB domain.

The inhibitory IGFBP7 polypeptides can be administered to a subject via any administration routes such as intravenous route. Alternatively, the inhibitory polypeptide can be administered to the subject via is administration of a polynucleotide encoding the inhibitory IGFBP7 polypeptide.

Also contemplated herein are inhibitory IGFBP7 polypeptides comprising one or more mutations that alloys the inhibitory IGFBP7 polypeptide to show preferential binding to CD93 over one or more other binding partners of IGFBP7 such as IGF-1, IGF-2, and IGF1R. In some embodiments, the inhibitors IGFBP7 polypeptide binds to CD93 with a greater affinity than for other one or more other binding partners of IGFBP7 such as IGF-1, IGF-2, and IGF1R. In some embodiments, the inhibitory IGFBP7 polypeptide binds to CD93 with a greater affinity as compared to wildtype IGFBP7.

In some embodiments, the methods described herein comprise administering to the subject an effective amount of an agent that reduces expression of CD93 or IGFBP7. In some embodiments, the agent is selected from the group consisting of: siRNA, shRNA, miRNA, antisense RNA, and a gene editing system.

In some embodiments, the subject suitable for the methods described herein is a human. In some embodiments, the subject is characterized by abnormal tumor vasculature. In some embodiments, the subject is characterized by dense or enriched blood vessels. In some embodiments, the subject was subjected to a prior therapy, such as a prior therapy comprising administering an inhibitory of the VEGF signaling pathway including an anti-VEGF antibody or an inhibitory polypeptide comprising one or more VEGFR domains. In some embodiments, the subject is characterized by high expression of CD93. In embodiments, the subject is characterized by high expression of IGFBP7. In some embodiments, the subject is characterized by high expression of VEGF. In some embodiments, the tumor discussed herein is solid tumor, such as a solid tumor can be: colorectal cancer, non-small cell lung cancer, glioblastoma, renal cell carcinoma, cervical cancer, ovarian cancer, fallopian tube cancer, peritoneal cancer, breast cancer, prostate cancer, bladder cancer, oral squamous cell carcinoma, head and neck squamous cell carcinoma, brain tumors, bone cancer, melanoma.

In some embodiments, prior to the administration of the CD93/IGFBP7 blocking agent, the presence and distribution of CD93 or IGFBP7 on vessels of the tissue (such as tumor vessels) of the subject will be assessed, e.g., to determine the relative level and activity of CD93 or IGFBP7 on vessels in the subject. A subject hose tissue vessels (such as tumor vessels) express CD93 or IGFBP7 (such as those express or express high levels of CD93 or IGFBP7) can be candidates for treatment with the CD93/IGFBP7 blocking agent. This can be accomplished by obtaining a sample tissue (such as tumor tissue), and testing e.g., using immunoassays, to determine the relative prominence of CD93 or IGFBP7 and optionally further other markers on the cells. In vivo imaging can also be used for detection of CD93 or IGFBP7 expression. Other methods can also be used to detect expression of CD93 and IGFBP7 include RNA-based methods. e.g., RT-PCR or Northern blotting.

The methods may involve multiple rounds of administration of the CD93/IGFBP7 blocking agent. In some embodiments, following an initial round of administration, the level and/or activity of CD93 or IGFBP7, in the subject may be re-measured, and, if still elevated, an additional round of administration can be performed. In this way, multiple rounds of the CD93/IGFBP7 blocking agent administration can be performed.

### Agent Inhibiting the IGFBP7/CD93 Signaling Pathway

The agent may be any of an antibody, a polypeptide, a peptide, a polynucleotide, a peptidomimetic, a natural product, a carbohydrate, an aptamer an avimer, an anticalin, a speigelmer, or a small molecule. Particular examples of what the agent may be are described below, and methods for identifying suitable agents feature in a subsequent aspect of the application. In some embodiments, the agent is a fusion protein (such as a fusion protein that comprises a half-life extending domain (e.g., a Fc domain)).

### CD93

CD93 is a type 1 transmembrane protein belonging to the gene family of C-type lectins and is known as the complement C1q receptor (C1qRp). CD93 consists of a C-type lectin-like domain (D1), five EGF-like repeats (D2), a mucin-like domain (D3), a transmembrane domain (D4), a cytoplasmic domain (D5), and a 79-amino acid DX domain localized between D1 and D2 [9]. CD93 is predominantly expressed on endothelial cells (ECs) and is implicated in promoting angiogenesis as a soluble growth factor and an EC adhesion molecule. Precious studies have shown that Multimerin 2 (MMRN2) interacts to CD93 to promote EC adhesion, migration, and in vitro angiogenesis. MMRN2, also called EndoGlyx-1, is an endothelial-specific member of the EDEN protein family and a component of the ECM. In tumor tissues, MMRN2 is found to express along tumor capillaries and co-expressed with CD93 in tumor neovasculature. See Galvagni et al., Matrix Biol. (2017) 64, 112-127, incorporated herein by reference in its entirety for all purposes.

The human CD93 gene is located at 20p11.21 and encodes a 652 amino acid residue polypeptide. The term “CD93 polypeptide” includes the meaning of a gene product of human CD93, including naturally occurring variants thereof. Human CD93 polypeptide includes the amino acid sequence found in Genbank Accession No NP_036204.2 and naturally occurring variants thereof. “Natural variants” include, for example, allelic variants. Typically, these will vary from the given sequence by only one or two or three, and typically no more than 10 or 20 amino acid residues. Typically, the variants have conservative substitutions. The CD93 polypeptide sequence from NP 036204.2 is shown as SEQ ID NO: 1. Natural variants of human CD93 include those with an A220V mutation, a V318A mutation or a P541 mutation.

CD93 described in the present application include any naturally occurring CD93 or variants thereof that have function of CD93. Also included are CD93 orthologues found in other species, such as in horse, bull, chimp, chicken, zebrafish, dog, pig, cow, sheep, rat, mouse, guinea pig or a primate.

### IGFBP7

Insulin-like growth factor (IGF)-binding protein (IGFRP) 7, also known as Mac25, IGFBP-rp1, tumor-derived adhesion factor (TAF), prostacyclin-stimulating factor (PSF), and angiomodulin (ACM), is a secreted extracellular matrix (ECM) protein belonging to IGFBP family (57, 58). Members of IGFBP family contain an IGF-binding (IB) domain at the N-terminus which binds to IGF1 and helps to modulate the bioavailability of IGF1 in the blood. IGFBP7 lacks the C-terminal domain, which functions to stabilize IGF1 binding, thus its affinity for IGF-1 is significantly lower than that of IGFBP1-6 (59). IGFBP7 was found to be expressed in many normal tissues and cancer cells; however, the exact role of IGFBP7 in cancer was controversial. On one hand, IGFBP7 was shown to be released from cancer cells, and to act as a tumor suppressor to trigger tumor apoptosis and suppress angiogenesis (60); IGF-1R was proposed as the receptor and IGFBP7 binding blocked the interaction between IGF-1 and IGF1R to inhibit expansion and aggressiveness of cancer stem-like cells (61, 62). Administration of IGFBP7 inhibited tumor growth in vivo, and IGFBP7−/− mice were susceptible to diethylnitrosamine-induced hepatocarcinogenesis (55, 63). On the other hand. IGFBP7 was shown to be upregulated in blood vessels of cancer tissues and was capable of promoting vascular angiogenesis (48, 64). IGFBP7 can be strongly induced by VEGF in vascular EC (48), and a synergistic effect between IGFBP7 and VEGF in angiogenesis has been reported (50). Each reference listed above is incorporated by reference in its entirety for all purposes.

The human IGFBP7 gene locates at 4q12 and encodes a polypeptide. One isoform of the polypeptide has 264 amino acid residues (SEQ ID NO: 2) that include a signal peptide domain (residues 1-26 of SEQ ID NO: 2), an insulin-binding domain (IB domain, residues 28-106 of SEQ ID NO: 2), a Kazal-like domain (residues 105-158 of SEQ ID NO: 2), and a Ig-like C2-type domain (residues 160-264 of SEQ ID NO: 2).

IGFBP7 described in the present application include any naturally occurring IGFBP7 or variants thereof that has e function of IGFBP7. Also included are IGFBP7 orthologues found in other species, such as in horse, bull, chimp, chicken, zebrafish, dog, pig, cow, sheep, rat, mouse, guinea pig or a primate.

### Anti-CD93 or Anti-IGFBP Antibodies

A. Anti-CD93 Antibodies

The methods described herein in some embodiments involve the use of anti-CD93 antibodies that specifically recognize CD93 and specifically blocks the interaction between CD93 and IGFBP7. The present application in one aspect also provides any of the novel anti-CD93 antibodies described herein.

In some embodiments, the CD93 recognized by the anti-CD93 antibody is a human CD93 In some embodiments, the human CD93 comprises or has the amino acid sequence of SEQ ID NO: 1 or a natural variant of human CD93. In some embodiments, the natural variant of human CD93 is derived from a tumor tissue.

In some embodiments, the anti-CD93 antibody binds to the IGFBP7 binding site on CD93. In some embodiments, the anti-CD93 antibody binds to a region on CD93 that is outside of the IGFBP7 binding site.

In some embodiments, the anti-CD93 antibody binds to the extracellular region of CD93. In some embodiments, the anti-CD93 antibody binds to the extracellular region of human CD93 (such as residues A24-K580 according to SEQ ID NO: 1).

In some embodiments, the anti-CD93 antibody binds to the C-type lectin domain of CD93. In some embodiments, the anti-CD93 antibody binds to the C-type lectin domain of human CD93 (such as residues T22-N174 according to SEQ ID NO: 1).

In some embodiments, the anti-CD93 antibody binds to long-loop region in the C-type lectin domain of CD93. In some embodiments, the anti-CD93 antibody binds to long-loop region in the C-type lectin domain of human CD93 (such as residues G96-C141 according to SEQ ID NO: 1). In some embodiments, the anti-CD93 antibody binds to less conserved residues in the C-type lectin domain or the long-loop region in the C-type lectin domain of CD93. For example, the anti-CD93 antibody binds to any one or more (such as about 2, 3, 4, 5, 6, 7, 8, 9, or 10) of residues selected from G96, Q98, R99, E100, K101, G102, K103, C104, L105, D106, P107, S108, L109, K112, S115, V117, G118, G120, E121, D122, T123, P124, Y125, S126, N127, H129, K130, E131, L132, R133, N134, S135, C136, H37, S138, K139, and R140 according to SEQ ID NO: 1. In some embodiments, the anti-CD93 antibody binds to a region of human CD93 that comprises or consists of residues F182-Y262 according to SEQ ID NO: 1. In some embodiments, the anti-CD93 antibody binds to F238 according to SEQ ID NO: 1.

In some embodiments, the anti-CD93 antibody binds to the DX domain between the C-type lectin-like domain (D1 domain) and the EGF-like domain (D2 domain). In some embodiments, the anti-CD93 antibody binds to the DX domain of human CD93 (such as residues I175-L256 or I175-S259 according to SEQ ID NO: 1). In some embodiments, the anti-CD93 antibody binds to F238 according to SEQ ID NO: 1.

In some embodiments, the anti-CD93 antibody binds to both the DX domain and the C-type lectin domain of CD93. In some embodiments, the anti-CD93 antibody binds to both F238 and the C-type lectin domain of human CD93 (such as residues T22-N174 according to SEQ ID NO: 1). In some embodiments, the anti-CD93 antibody binds to both F238 and long-loop region in the C-type lectin domain of human CD93 (such as residues G96-C141 according to SEQ ID NO 1). In some embodiments, the anti-CD93 antibody binds to both F238 and any one or more (such as about 2, 3, 4, 5, 6, 7, 8, 9, or 10) of residues selected from G96, Q98, R99, E100, K101, G102, K103, C104, L105, D106, P107, S108, I109, K112, S115, V117, G118, G120, E121, D122, T123, P124, Y125, S126, N127, H129, K130, E131, L132, R133, N134, S135, C136, I137, S138, K139, and R140 according to SEQ ID NO: 1.

In some embodiments, the anti-CD93 antibody binds to the EGF-like region of CD93. In some embodiments, the anti-CD93 antibody binds to the EGF-like region of human CD93 (such as residues C257-M469 or P260-T468 according to SEQ ID NO: 1).

In some embodiments, the anti-CD93 antibody also blocks interaction between CD93 and MMNR2. In some embodiments, the anti-CD93 antibody binds to the same epitope of CD93 from the epitope that MMNR2 binds to. In some embodiments, the anti-CD93 antibody binds to a distinct epitope of CD93 from the epitope that MMNR2 binds to.

In some embodiments, the anti-CD93 antibody does not block the interaction between CD93 and MMNR2.

In some embodiments, the anti-CD93 antibody is a poll clonal antibody In some embodiments, the anti-CD93 antibody is a monoclonal antibody.

In some embodiments, the anti-CD93 antibody is an anti-human CD93 antibody.

In some embodiments, the anti-CD93 antibody is humanized or chimeric.

In some embodiments, the anti-CD93 antibody binds to CD93 competitively against mAb MM01 (SinoBiological), R3 (SinoBiological) or 273107 (SinoBiological). In some embodiments, the anti-CD93 antibody binds to an epitope that overlaps or substantially overlaps with that of mAb MM01 (SinoBiological), R3 (SinoBiological) or 273107 (SinoBiological). In some embodiments, the anti-CD93 antibody does not bind to an epitope that substantially overlaps with that of mAb MM01 (SinoBiological), R3 (SinoBiological) or 273107 (SinoBiological). In some embodiments, “substantially overlap” described above refers to the scenario that at least about 50%, 60%, 70%, 80%, or 90% of the residues on CD93 that the anti-CD93 antibody binds to overlap with the residues that MM01 (SinoBiological), R3 (SinoBiological) or 273107 (SinoBiological) binds to. In some embodiments, the anti-CD93 antibody binds to at least one, two, three, four, five, six, seven, eight, nine or ten of residues on CD93 that MM01 (SinoBiological), R3 (SinoBiological) or 273107 (SinoBiological) binds to.

In some embodiments, the anti-CD93 antibody does not bind to CD93 competitively against mAb MM02 (SinoBiological). In some embodiments, the anti-CD93 antibody does not bind to CD93 competitively against mAb R004 (SinoBiological).

In some embodiments, the anti-CD93 antibody binds to CD93 competitively against mAb 7C10. In some embodiments, the anti-CD93 antibody binds to an epitope that overlaps or substantially overlaps with that of 7C10. In some embodiments, the anti-CD93 antibody does not bind to an epitope that substantially overlaps with that of 7C10. In some embodiments, the anti-CD93 antibody binds to at least one, two, three, four, five, six, seven, eight, nine or ten of residues on CD93 that 7C10 binds to

In some embodiments, the anti-CD93 antibody is anti-human CD93 monoclonal antibody selected from the group consisting of EPR5386 (abcam), 3D12 (sigma-aldrich), 1A4 (sigma-aldrich), 1A10E10, 2F7D11, R139, R3, mNI-11, X-2, and MM01.

In some embodiments, the anti-human CD93 antibody is mAb MM01 or a humanized version thereof.

In some embodiments, the anti-CD93 antibody is a full-length antibody or immunoglobulin derivatives. In some embodiments, the anti-CD93 antibody is an antigen-binding fragment, for example an antigen-binding fragment selected from the group consisting of a single-chain Fv (scFV), a Fab, a Fab′, a F(ab′)2, an Fv fragment, a disulfide stabilized Fv fragment (dsFv), a (dsFv)₂, a V_{H}H, a Fv-Fc fusion, a scFV-Fc fusion, a scFv-Fv fusion, a diabody, a tribody, and a tetrabody. In some embodiments, the anti-CD93 antibody is a scFV. In some embodiments, the anti-CD93 antibody is a Fab or Fab′. In some embodiments, the anti-CD93 antibody is chimeric, human, partially humanized, fully humanized, or semi-synthetic. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the anti-CD93 antibody comprises an Fc fragment. In some embodiments, the Fc fragment is selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG.

B. Anti-IGFBP7 Antibodies

The methods described herein in some embodiments involve the use of anti-IGFBP7 antibodies that specifically recognize IGFBP7 and specifically blocks interaction between CD93 and IGFBP7. The present application in one aspect also provides any of the novel anti-IGFBP7 antibodies described herein.

In some embodiments, the IGFBP7 recognized by the anti-IGFBP7 antibody is a human IGFBP7. In some embodiments, the IGFBP7 is a mouse IGFBP7.

In some embodiments, the anti-IGFBP7 antibody binds to the CD93 (such as a human CD93) binding site on IGFBP7. In some embodiments, the anti-IGFBP7 antibody binds to a region on IGFBP7 that is outside of the CD93 binding site.

In some embodiments, the anti-IGFBP7 antibody binds to the insulin-binding domain (“IB domain”) of the IGFBP7. In some embodiments, the anti-IGFBP7 antibody binds to the IB domain of the human IGFBP7 (such as residues S28-G106 according to SEQ ID NO: 2).

In some embodiments, the anti-IGFBP7 antibody binds to the Kazal-like domain of the IGFBP7. In some embodiments, the anti-IGFBP7 antibody binds to the Kazal-like domain of a human IGFBP7 (such as residues P105-Q158 according to SEQ ID NO: 2).

In some embodiments, the anti-IGFBP7 antibody binds to the Ig-like C2 domain of the IGFBP7. In some embodiments, the anti-IGFBP7 antibody binds to the Ig-like C2 domain of a human IGFBP7 (such as residues P160-T264 according to SEQ ID NO: 2).

In some embodiments, the anti-IGFBP7 antibody does not specifically bind to any one or more of IGFBP1, IGFBP2, IGFBP3, IGFBP4, IGFBP5, IGFBP6, IGFBPL1, KAZALD1, HTRA1, WISP1, WISP3, NOV, CYR61, CTGF, and ESM1. In some embodiments, the anti-IGFBP7 antibody does not specifically bind to any one molecule selected from the group consisting of IGFBP1, IGFBP2, IGFBP3, IGFBP4, IGFBP5, IGFBP6, IGFBPL1, KAZALD1, HTRA1, WISP1, WISP3, NOV, CYR61, CTGF, and ESM1.

In some embodiments, the anti-IGFBP7 antibody also blocks interaction between IGFBP7 and IGF-1, IGF-2, and/or IGF1R.

In some embodiments, the anti-IGFBP7 antibody does not block the interaction between IGFBP7 and IGF-1, IGF-2, and/or IGF1R.

In some embodiments, the anti-IGFBP7 antibody is a polyclonal antibody. In some embodiments, the anti-IGFBP7 antibody is a monoclonal antibody.

In some embodiments, the anti-IGFBP7 antibody is an anti-human IGFBP7 antibody.

In some embodiments, the anti-IGFBP7 antibody is humanised or chimeric.

In some embodiments, the anti-IGFBP7 antibody binds to IGFBP7 competitively with mAb R003 (SinoBiological), MM01 (SinoBiological), R065 (SinoBiological) or R115 (SinoBiological). In some embodiments, the anti-IGFBP7 antibody binds to an epitope that overlaps with that of mAb R003 (SinoBiological), MM01 (SinoBiological), R065 (SinoBiological) or R115 (SinoBiological). In some embodiments, the anti-IGFBP7 antibody binds to at least one, two, three, four, five, six, seven, eight, nine or ten of residues on IGFBP7 that R003 (SinoBiological), MM01 (SinoBiological), R065 (SinoBiological) or R115 (SinoBiological) binds to.

In some embodiments, the anti-IGFBP7 antibody binds to IGFBP7 competitively with mAb 2C6. In some embodiments, the anti-IGFBP7 antibody binds to an epitope that overlaps with that of mAb 2C6. In some embodiments, the anti-IGFBP7 antibody binds to at least one, two, three, four, five, six, seven, eight, nine or ten of residues on IGFBP7 that 2C6 binds to.

In some embodiments, the anti-IGFBP7 antibody is anti-human IGFBP7 monoclonal antibody selected from the group consisting of mAb AEDO-9 (clone name, same for the following antibodies) (Bosterbio). ID9E7 (LifeSpan BioSciences), 5A4A9) (LifeSpan BioSciences), 192520 (R&D systems), H3 (Santa Cruz/Biotechnology), 40012B (R&D Systems), EPR11912(B) (Abcam), MM0346-3N37 (Abcam), 01 (i.e., MM01, Sino Biological), 003 (i.e., R003, Sino Biological). In some embodiments, the anti-human IGFBP7 monoclonal antibody is mAb 003 (i.e., R003, Sino Biological) or a humanized version thereof.

In some embodiments, the anti-IGFBP antibody is a full-length antibody or immunogloulin derivatives. In some embodiments, the anti-IGFRP antibody is an antigen-binding fragment, for example an antigen-binding fragment selected from the group consisting of a single-chain Fv (scFv), a Fab, a Fab′, a F(ab′)2, an Fv fragment, a disulfide stabilized by fragment (dsFv), a (dsFv)₂, a V_{H}H, a Fv-Fc fusion, a scFv-Fc fusion, a scFv-Fv fusion, a diabody, a tribody, and a tetrabody. In some embodiments, the anti-IGFRP antibody is an scFv. In some embodiments, the anti-IGFBP antibody is a Fab or Fab′. In some embodiments, the anti-IGFRP antibody is chimeric, human, partially humanized, fully humanized, or semi-synthetic. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the anti-IGFRP antibody comprises an Fc fragment. In some embodiments, the Fc fragment is selected from the group consisting of Fc fragments from IgG, IgA, IgD, IgE, IgM, and combinations and hybrids thereof in some embodiments, the Fc fragment is derived from a human IgG. In some embodiments, the Fc fragment comprises the Fc region of human IgG1, IgG2, IgG3, IgG4, or a combination or hybrid IgG.

Competition Assays and Epitope Mapping

The descriptions below about competition assays and epitope mapping use anti-IGFBP7 antibody as examples for demonstration. It similarly applies to anti-CD93 antibodies described above.

Competition can be assessed by, for example, a flow cytometry test. In such a test, cells bearing a given IGFBP7 polypeptide that has the IGFBP7 can be incubated first with an antibody (e.g., mAb 2C6) and then with the test antibody labeled with a fluorochrome or biotin. The antibody is said to compete with 2C6 or binds to IGFBP7 competitively with 2C6 if the binding obtained upon pre-incubation with a saturating amount of 2C6 is about 80%, preferably about 50%, about 40% or less (e.g. about 30%, 20% or 10%) of the binding (as measured by mean of fluorescence) obtained by the antibody without pre-incubation with 2C6. Alternatively, an antibody is said to compete with 2C6 if the binding obtained with a labeled 2C6 antibody (by a fluorochrome or biotin) on cells pre-incubated with a saturating amount of test antibody is about 80%, preferably about 50%, about 40%, or less (e.g., about 30%, 20% or 10%) of the binding obtained without pre-incubation with the test antibody.

A simple competition assay in which a test antibody is pre-adsorbed and applied at saturating concentration to a surface onto which IGFBP7 is immobilized may also be employed. The surface in the simple competition assay is preferably a BIACORE chip (or other media suitable for surface plasmon resonance analysis). The control antibody (e.g., 2C6) is then brought into contact with the surface at an IGFBP7-saturating concentration and the IGFBP7 and surface binding of the control antibody is measured. This binding of the control antibody is compared with the binding of the control antibody to the IGFBP7-containing surface in the absence of test antibody. In a test assay, a significant reduction in binding of the IGFBP7-containing surface by the control antibody in the presence of a test antibody indicates that the test antibody recognizes substantially the same epitope as the control antibody such that the test antibody “cross-reacts” with the control antibody. Any test antibody that reduces the binding of control (such as 2C6) antibody to an IGFBP7 by at least about 30% or more, preferably about 40%, can be considered to be an antibody that binds to substantially the same epitope or determinant as a control (e.g., 2C6). Preferably, such a test antibody will reduce the binding of the control antibody (e.g., 2C6) to the IGFBP7 by at least about 50% (e.g., at least about 60%, at least about 70%, or more). It will be appreciated that the order of control and test antibodies can be reversed; that is, the control antibody can be first bound to the surface and the test antibody is brought into contact with the surface thereafter in a competition assay. Preferably, the antibody having higher affinity for the IGFBP7 is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein reference in its entirety for all purposes.

Preferably, monoclonal antibodies that recognize an IGFBP7 epitope will react with an epitope that is present on a substantial percentage of or even all relevant IGFBP7 alleles.

In preferred embodiments, the antibodies will bind to IGFBP7-expressing cells from a subject or subjects with a disease characterized by expression of IGFBP7-positive cells, i.e. a subject that is a candidate for treatment with one of the herein-described methods using an anti-IGFBP7 antibody of the application. Accordingly, once an antibody that specifically recognizes IGFBP7 on cells is obtained, it can be tested for its ability to bind to IGFBP7-positive cells (e.g. cancer cells). In particular, prior to treating a patient with one of the present antibodies, it will be beneficial to test the ability of the antibody to bind malignant cells taken from the patient, e.g. in a blood sample or tumor biopsy, to maximize the likelihood that the therapy will be beneficial in the patient. In one embodiment, the antibodies of the application are validated in an immunoassay to test their ability to bind to IGFBP7-expressing cells, e.g. malignant cells. For example, a tumor biopsy is performed and tumor cells are collected. The ability of a given antibody to bind to the cells is then assessed using standard methods well known to those in the art. Antibodies that are found to bind to a substantial proportion (e.g., 20%, 30%, 40%, 50%, 60%, 70%, 80% or more) of cells known to express IGFBP7, e.g. tumor cells, from a significant percentage of subjects or patients (e.g., 5%, 10%, 20% 30%, 40%, 0.50% or more) are suitable for use in the present invention, both for diagnostic purposes to determine the presence or level of malignant cells in a patient or for use in the herein-described therapeutic methods, e.g., for use to increase or decrease malignant cell number or activity. To assess the binding of the antibodies to the cells, the antibodies can be either directly or indirectly labeled. When indirectly labeled, a secondary, labeled antibody is typically added.

Determination of whether an antibody binds within an epitope region can be carried out in ways known to the person skilled in the art. As one example of such mapping characterization methods, an epitope region for an anti-IGFBP7 antibody may be determined by epitope “foot-printing” using chemical modification of the exposed amines/carboxy/s in the IGFBP7 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry. Vol. 267 (2) pp. 252-259 (1999); Engen, J. R, and Smith, D. L. (2001) Anal. Chem. 73, 256A-265A, each of which is incorporated herein by reference in their entirety for all purposes. Another example of a suitable epitope identification technique is nuclear magnetic resonance epitope napping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop 2004; (44); 149-67; Huang et al., Journal of Molecular Biology, Vol. 281 (1) pp. 61-67 (1998), and Saito and Patterson, Methods. 1996 June; 9 (3): 516-24, each of which is incorporated herein by reference in their entirety for all purposes.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801, each of which is incorporated herein by reference in their entirety for all purposes. Protease digestion techniques also can be useful in the context of epitope mapping and identification. Antigenic determinant-relevant regions/sequences can be determined by protease digestion, e.g. by using trypsin in a ratio of about 1:50 to IGFBP7 or o/n digestion at and pH 7-8, followed by mass spectrometry (MS) analysis for peptide identification. The peptides protected from trypsin cleavage by the anti-IGFBP7 binder can subsequently be identified by comparison of samples subjected to trypsin digestion and samples incubated with antibody and then subjected to digestion by e.g. trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the IGFBP7 polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is re-placed with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells. Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fagerstam et al., Journal of Molecular Recognition 1990; 3:208-14; Nice et al., J. Chromatogr. 1993; 646:159-168; Leipert et al., Angew. Chem Int Ed 1998; 37:3308-3311; Kroger et al., Biosensors and Bioelectronics 2002; 17:037-944.

It should also be noted that an antibody (the first antibody) binding the same or substantially the same epitope as an antibody of the application (the second antibody) can be identified in one or more of the exemplary competition assays described herein. In some embodiments, the first antibody binding to substantially the same epitope as the second antibody refers to the scenario that the residues that the first antibody binds to have an overlap of at least about 50%, 60%, 70%, 80%, or 90% with the residues that the second antibody binds to.

Agents Comprising anti-CD93 Antibody or Anti-IGFBP7 Antibody

A. Anti-CD93 or Anti-IGFBP7 Fc Elusion Proteins

In some embodiments, the agent that comprises an anti-CD93 antibody or anti-IGFBP7 antibody as described herein is a fusion protein. In some embodiments, the anti-CD93 and/or anti-IGFBP7 antibody (such as an anti-CD93 and/or anti-IGFBP7 antibody fragment) is fused to an Fc fragment via a linker (such as peptide linker). Any of the anti-CD93 or anti-IGFBP7 antibodies described in the “anti-CD93 or anti-IGFBP7 antibodies” section can be employed in the anti-CD93 or anti-IGFBP7 Fc fusion protein.

1. Fc Fragment

The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md. 1991.

In some embodiments, the Fc fragment comprises an immunoglobulin heavy chain constant region comprising a hinge region, a CH2 domain and/or a CH3 domain. The term “hinge region” or “hinge sequence” as used herein refers to the amino acid sequence located between the linker and the CH2 domain. In some embodiments, the fusion protein comprises an Fc fragment comprising a hinge region. In some embodiments, the hinge region comprises the amino acid sequence CPPCP (SEQ ID NO: 3), a sequence found in the native IgG1 hinge region, to facilitate dimerization. In some embodiments, the Fc fragment of the fusion protein starts at the hinge region and extends to the C-terminus of the IgG heavy chain. In some embodiments, the fusion protein comprises an Fc fragment that does not comprise the hinge region. In some embodiments, the Fc fragment comprises a human IgG heavy chain hinge region (starting at Cys226), an IgG CH2 domain and/or IgG CH3 domain.

In some embodiments, the fusion protein comprises an Fc fragment selected from the group consisting of Fc fragments from IgG, IgA, IgD, IgF, IgM, and combinations and hybrids thereof. In some embodiments, the be fragment is derived from a human IgG. In some embodiments, the Fc fragment comprises the Fc region of human IgG1, IgG2, IgG3, IgG4, or a combination or hybrid IgG. In some embodiments, the Fc fragment is an IgG1 Fc fragment. In some embodiments, the Fc fragment comprises the CH2 and CH3 domains of IgG1. In some embodiments, the Fc fragment is an IgG4 Fc fragment. In some embodiments, the Fc Fragment comprises the CH2 and CH3 domains of IgG4. IgG4 Fc is known to exhibit less effector activity than IgG1 Fc, and thus may be desirable for some applications. In some embodiments, the Fc fragment is derived from of a mouse immunoglobulin.

In some embodiments, the IgG CH2 domain starts at Ala231. In some embodiments, the IgG CH3 domain starts at Gly341. In some embodiments, the C-terminus Lys residue of human IgG is absent. In some embodiments, conservative amino acid substitution(s) is/are made in the Fc region without affecting the desired structure and/or stability of Fc.

Additionally, anti-CD93 or anti-IGFBP7-Fc fusion proteins comprising any of the Fc variants described below, or combinations thereof, are contemplated. In some embodiments, the Fc fragment comprises sequence that has been altered or otherwise changed so that it has enhanced antibody dependent cellular cytotoxicity (ADCC) or complement dependent cytotoxicity (CDC) effector function.

Heterodimerization of non-identical polypeptides in the anti-CD93 or anti-IGFBP7-Fc fusion protein can be facilitated by methods known in the art, including without limitation, heterodimerization by the knob-into-hole technology. The structure and assembly method of the knob-into-hole technology can be found in, e.g., U.S. Pat. Nos. 5,821,333, 7,642,228, US 2011/0287009 and PCT/US2012/059810, hereby incorporated by reference in their entireties for all purposes. This technology was developed by introducing a “knob” (or a protuberance) by replacing a small amino acid residue with a large one in the CH3 domain of one Fc, and introducing a “hole” (or a cavity) in the CH3 domain of the other Fc by replacing one or more large amino acid residues with smaller ones. In some embodiments, one chain of the Fc fragment in the fusion protein comprises a knob, and the second chain of the Fc fragment comprises a hole.

The preferred residues for the formation of a knob are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the knob has a small side chain volume, such as alanine, asparagine, aspartic acid, glycine, serine, threonine or saline. Exemplary amino acid substitutions in the CH3 domain of an IgG for forming the knob include without limitation the T1366W, T366Y or F405W substitution.

The preferred residues for the formation of a hole are usually naturally occurring amino acid residues and are preferably selected from alanine (A), serine (S), threonine (T) and saline (V). In one embodiment, the original residue for the formation of the hole has a large side chain volume, such as tyrosine, arginine, phenylalanine or tryptophan. Exemplary amino acid substitutions in the CH3 domain of an IgG for generating the hole include without limitation the T366S, L368A, F405A, Y407A, Y407T and Y407V substitutions. In certain embodiments, the knob comprises T366W substitution, and the hole comprises the T366S/L368A/Y 407V substitutions. It is understood that other modifications to the Fc region known in the art that facilitate heterodimerization are also contemplated and encompassed by the instant application.

The methods that involve agents such as variants of isolated anti-CD93 or anti-IGFBP7-Fc fusion protein, e.g., a full-length anti-CD93 or anti-IGFBP7 antibody variant) comprising any of the variants described herein (e.g., Fc variants, effector function variants, glycosylation variants, cysteine engineered variants), or combinations thereof, are contemplated.

2. Linkers

In some embodiments, the anti-CD93 or anti-IGFBP7-Fc fusion proteins described herein comprise an anti-CD93 or anti-IGFBP7 antibody described herein fused to an Fc fragment via a linker.

The length, the degree of flexibility and/or other properties of the linker used in the anti-CD93 or anti-IGFBP7-Fc fusion proteins may have some influence on properties, including but not limited to the affinity, specificity or avidity of the anti-CD93 or anti-IGFBP7 antibody, and/or affinity, specificity or avidity for one or more particular antigens or epitopes present on CD93 and/or IGFBP7. For example, longer linkers may be selected to ensure that two adjacent antibody moieties do not sterically interfere with one another. In some embodiments, a linker (such as peptide linker) comprises flexible residues (such as glycine and serine) so that the adjacent antibody moieties are free to move relative to each other. For example, a glycine-serine doublet can be a suitable peptide linker. In some embodiments, the linker is a non-peptide linker. In some embodiments, the linker is a peptide linker. In some embodiments, the linker is a non-clear able linker. In some embodiments, the linker is a cleavable linker.

Other linker considerations include the effect on physical or pharmacokinetic properties of the resulting anti-CD93 or anti-IGFBP7-Fc fusion protein, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

a. Non-Peptide Linkers

Any one or all of the linkers described herein can be accomplished by any chemical reaction that will bind the two molecules so lone as the components or fragments retain their respective activities, i.e. binding to target CD93 or IGFBP7, binding to FcR, and/or ADCC/CDC. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules, such as an Fc fragment to the anti-CD93 or anti-IGFBP7 antibody of the present invention. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (sere Killen and Lindstrom. Jour. Immun. 133:1335-2549 (1984); Jansen el Immunological Reviews 62:185-216 (1982); and Vitetta et al., Science 238:1098 (1987), each incorporated by reference in their entirety for all purposes).

Linkers that can be applied in the present application are described in the literature (see, for example. Ramakrishnan. S, et al., Cancer Res. 44:201-208 (1984) describing use of MBS (M-maleimidobenzoyl-N-hydroxysuccinimide ester), incorporated by reference in its entirety for all purposes). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide; Pierce Chem. Co., Cat. #24510) conjugated to EDC.

The linkers described above contain components that have different attributes, thus leading to anti-CD93 or anti-IGFBP7-Fc fusion proteins with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

b. Peptide Linkers

Any one or all of the linkers described herein can be peptide linkers. The peptide linker may have a naturally occurring sequence, or a non-naturally occurring sequence. For example, a sequence derived from the hinge region of heavy chain only antibodies may be used as the linker. See, for example, WO1996/34103, incorporated by reference in its entirety for all purposes. In some embodiments, the peptide linker comprises the amino acid sequence of CPPCP (SEQ ID NO: 3), a sequence found in the native IgG1 hinge region.

The peptide linker can be of any suitable length. In some embodiments, the length of the peptide linker is any of about 1 aa to about 10 aa, about 1 aa to about 20 aa, about 1 aa to about 30 aa, about 5 aa to about 15 aa, about 10 aa to about 25 aa, about 5 aa to about 30 aa, about 10 aa to about 30 aa, about 30 aa to about 50 aa, about 50 aa to about 100 aa, or about 1 aa to about 100 aa.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273). Cheadle et al. (Mol Immunol (1992) 29, 21-30) and Raag and Whitlow (FASEB (1995) 9(1), 73-80, each incorporated by reference in their entirety for all purposes). A particularly preferred amino acid in context of the “peptide linker” is Gly. Furthermore, peptide linkers that also do not promote any secondary structures are preferred. The linkage of the molecules to each other can be provided by, e.g., genetic engineering. Methods for preparing fused and operatively linked antibody constructs and expressing them in mammalian cells or bacteria are well-known in the art (e.g. WO 99/54440, Ausubel, Current Protocols in Molecular Biology, Green Publishing Associates and Wiley Interscience. N. Y. 1989 and 1994 or Sambrook et al., Molecular Cloning: A Laboratory Manual, Cold Spring Harbor Laboratory Press, Cold Spring Harbor, N.Y., 2001, each incorporated h reference in their entirety for all purposes).

In some embodiments, the peptide linker is a stable linker, which is not cleavable by protease, such as by Matrix metalloproteinases (MMPs).

In some embodiments, the peptide linker tends not to adopt a rigid three-dimensional structure, but rather provide flexibility to a polypeptide (e.g., first and/or second components), such as providing flexibility between the anti-CD93 or anti-IGFBP7 antibody and the Fc fragment. In some embodiments, the peptide linker is a flexible linker. Exemplary flexible linkers include glycine polymers (G)ₙ (SEQ ID NO: 4), glycine-serine polymers (including, for example, (GS)ₙ (SEQ ID NO: 5), (GSGGS)ₙ (SEQ ID NO: 6), (GGGGS)ₙ (SEQ ID NO 7), and (GGGS)ₙ (SEQ ID NO 8), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an anti-CD93 or anti-IGFBP7-Fc fusion protein can include linkers that are all or partially flexible, such that the linker can include a flexible linker portion as well as one or more portions that confer less flexible structure to provide a desired fusion protein structure.

In some embodiments, the anti-CD93 or anti-IGFBP7 antibody (such as the anti-CD93 or anti-IGFBP7 antibody fragment) and the Fc fragment are linked together by a linker of sufficient length to enable the anti-CD93 or anti-IGFBP7-Fc fusion protein to fold in such away as to permit binding to target CD93 or IGFBP7, as well as to FcR. In some embodiments, the linker comprises the amino acid sequence of SRGGGGSGGGGSGGGGSLEMA (SEQ ID NO: 9). In some embodiments, the linker is or comprises a (GGGGS)ₙ (SEQ ID NO: 13) sequence, wherein n is equal to 1, 2, 3, 4, 5, 6, 7, 8, 9, 10 or more. In some embodiments, the linker comprises the amino acid sequence of TSGGGGS (SEQ ID NO: 10). In some embodiments, the linker comprises the amino acid sequence of GEGTSTGSGGSGGSGGAD (SEQ ID NO: 11).

Natural linkers adopt various conformations in secondary structure, such as helical, β-strand, coil/bend and turns, to exert their functions. Linkers in an α-helix structure might serve as rigid spacers to effectively separate protein domains, thus reducing their unfavorable interactions. Non-helical linkers with Pro-rich sequence could increase the linker rigidity and function in reducing inter-domain interference. In some embodiments, the anti-CD93 or anti-IGFBP7 antibody (such as antibody fragment) and the Fc fragment (or an antibody comprising an Fc fragment) is linked together by an α-helical linker with an amino acid sequence of A(EAAAK)₄A (SEQ ID NO: 12).

B. Multi-Specific Anti-CD93 or Anti-IGFBP7 Molecules

Multi-specific molecules are molecules that have binding specificities for at least two different antigens or epitopes (e.g., bispecific antibodies have binding specificities for two antigens or epitopes). Multi-specific molecules with more than two valences and/or specificities are also contemplated. For example, trispecific antibodies can be prepared (Tutt et al. J. Immunol. 147; 60 (1991)). It is to be appreciated that one of skill in the art could select appropriate features of subject multi-specific molecules described herein to combine with one another to form a multi-specific anti-CD93 or anti-IGFBP7 molecule of the application.

In some embodiments, the agent that blocks interaction between CD93 and IGFBP7 comprise a multi-specific (e.g., bispecific) anti-CD93 or anti-IGFBP7 molecule comprising an anti-CD93 or anti-IGFBP7 antibody according to any one of the anti-CD93 or anti-IGFBP7 antibodies described herein, and a second binding moiety (such as a second antibody) specifically recognizing a second antigen. In some embodiments, the multi-specific anti-CD93 or anti-IGFBP7 molecule comprises an anti-CD93 or anti-IGFBP7 antibody and a second antibody specifically recognizing a second antigen.

In some embodiments, the multi-specific anti-CD93 or anti-IGFBP7 molecule is, for example, a diabody (db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g., a bispecific T cell engager), a tandem tri-scFv, a tri(a)body, a bispecific Fab2, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fc, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a heteromultimeric antibody, or a heteroconjugate antibody.

In some embodiments, the agent comprises an anti-CD93 and anti-IGFBP7 antibody. In some embodiments, the agent is a bispecific antibody.

In some embodiments, the agent that blocks interaction between CD93 and IGFBP7 comprise a multi-specific (e.g., bispecific) anti-CD93 molecule comprising a first anti-CD93 antibody that specifically binds to a first epitope of CD93 and a second anti-CD93 antibody that specifically binds to a second epitope of CD93. In some embodiments, one or both of the first and second epitopes overlaps or substantially overlaps with that of mAb MM01 or mAb 7C10. In some embodiments, one or both of the first antibody and second antibody binds to CD93 competitively against mAb MM01 or mAb 7C10. In some embodiments, one or both of the first antibody and second antibody also blocks interaction between CD93 and MMRN2. In some embodiments, one or both of the first antibody and second antibody does not block the interaction between CD93 and MMRN2. In some embodiments, one or both of the first antibody and second antibody binds to a region on CD93 that is outside of the IGFBP7 binding site.

In some embodiments, the agent that blocks interaction between CD93 and IGFBP7 comprise a multi-specific (e.g., bispecific) anti-IGFBP7 molecule comprising a first anti-IGFBP7 antibody that specifically binds to a first epitope of IGFBP7 and a second anti-IGFBP7 antibody that specifically binds to a second epitope of IGFBP7. In some embodiments, one or both of the first and second epitopes overlaps or substantially overlaps with that of mAb R003 or mAb 2C6. In some embodiments, one or both of the first antibody and second antibody bind to IGFBP7 competitively against mAb R003 or mAb 2C6.

### Inhibitory CD93 or IGFBP7 Polypeptides

A. Inhibitory CD93 Polypeptides

The methods described herein in some embodiments involve use of polypeptides that block the interaction between CD93 and IGFBP7 comprising the extracellular domain of CD93 or a variant thereof (“inhibitory CD93 polypeptide”). The present application in one aspect provides novel and non-naturally occurring inhibitory CD93 polypeptides described herein. In some embodiments, the inhibitory CD93 polypeptide is a soluble polypeptide.

In some embodiments, the inhibitory CD93 polypeptide is membrane bound. In some embodiments, the membrane bound inhibitory CD93 polypeptide binds to IGFBP7 but does not trigger CD93/IGFBP7 signaling. In some embodiments, the membrane bound inhibitory CD93 polypeptide binds to IGFBP7 and attenuates CD93/IGFBP7 signaling. In some embodiments, the membrane bound inhibitory CD93 polypeptide is introduced by a gene editing system or an mRNA delivery vehicle.

In some embodiments, the inhibitory CD93 polypeptide comprises the extracellular domain of CD93 (such as human CD93) or a variant thereof. In some embodiments, the inhibitory CD93 polypeptide comprises an amino acid sequence of residues A24-K580 of SEQ ID NO: 1 or variant thereof having at least about 80% (such as about 85%, 90%, 91%, 92%, 93%, 94%, 95%, 96%, 97%, 98%, or 99%) sequence identity to residues A24-K580 of SEQ ID NO: 1. In some embodiments, the inhibitory CD93 polypeptide further comprises a F238 residue, wherein the amino acid numbering is based on SEQ ID NO: 1.

In some embodiments, the inhibitory CD93 polypeptide comprises the C-type lectin domain of CD93 (such as human CD93) or a variant thereof. In some embodiments, the inhibitory CD93 polypeptide comprises an amino acid sequence of residues T22-N174 of SEQ ID NO: 1 or variant thereof having at least about 80% (such as about 85%, 90% 91%, 92%, 93%, 94%, 95%, 96%, 97%, 98%, or 99%) sequence identity to residues T22-N174 of SEQ ID NO: 1. In some embodiments, the inhibitory CD93 polypeptide further comprises a F238 residue, wherein the amino acid numbering is based on SEQ ID NO: 1.

In some embodiments, the inhibitory CD93 polypeptide comprises a long-loop region in the C-type lectin domain of CD93 (such as human CD93) or a variant thereof. In some embodiments, the inhibitory CD93 polypeptide comprises an amino acid sequence of residues G96-C141 of SEQ ID NO. 1 or variant thereof having at least about 80% (such as about 85%, 90%, 91%, 92%, 93%, 94%, 95%, 96%, 97%, 98%, or 99%) sequence identity to residues G96-C141 of SEQ ID NO 1. In some embodiments, the inhibitory CD93 polypeptide further comprises at least one or more (such as about at least 10, 15, 20, 25, 30, 35 or all) of residues selected from G96, Q98, R99, E100, K101, G102, K103, C104, L105, D106, P107, S108, L109, K112, S115, V117, G118, G120, E121, D122, T123, P124, Y125, S126, N127, H129, K130, E131, L132, R133, N134, S135, C136, I137, S138, K139, and R140, wherein the amino acid numbering is based on SEQ ID NO: 1.

In some embodiments, the inhibitory CD93 polypeptide comprises the DX domain between the C-type lectin-like domain (D1 domain) and the EGF-like domain (D2 domain) of CD93 (such as human CD93) or a variant thereof. In some embodiments, the inhibitory CD93 polypeptide comprises an amino acid sequence of residues I175-L256, and I175-L259 of SEQ ID NO: 1 or variant thereof having at least about 80% (such as about 85%, 90%, 91%, 92%, 93%, 94%, 95%, 96%, 97%, 98%, or 99%) sequence identity to residues I175-L256, and I175-L250 of SEQ ID NO: 1.

In some embodiments, the inhibitory CD93 polypeptide comprises an amino acid sequence of any one of residues F182-Y262, I175-L256, and/or I175-L259 of SEQ ID NO: 1 or a variant thereof having at least about 80% (such as about 85%, 90%, 91%, 92%, 93%, 94%, 95%, 96%, 97%, 98%, or 99%) sequence identity to the sequence of any one of residues F182-Y262, I175-L256, and I175-L259 of SEQ ID NO: 1. In some embodiments, the inhibitory CD93 polypeptide further comprises a F238 residue based upon SEQ ID NO:1. In some embodiments, the inhibitory CD93 polypeptide further comprises at least one or more (such as about at least 10, 15, 20, 25, 30, 35 or all) of residues selected from G96, Q98, R99, E100, K101, G102, K103, C104, L105, D106, P107, S108, L109, K112, S115, V117, G118, G120, E121, D122, T123, P124, Y125, S126, N127, H129, K130, E131, L132, R133, N134, S135, C136, I137, S138, K139, and R140, wherein the amino acid numbering is based on SEQ ID NO:1.

In some embodiments, the inhibitory CD93 polypeptide comprises an amino acid sequence of residues T22-Y262 of SEQ ID NO: 1 or variant thereof having at least about 80% (such as about 85%, 90%, 91%, 92%, 93%, 94%, 95%, 96%, 97%, 98%, or 99%) sequence identity to residues T22-Y262 of SEQ ID NO: 1. In some embodiments, the inhibitory CD93 polypeptide further comprises a F238 residue based upon SEQ ID NO: 1. In some embodiments, the inhibitory CD93 polypeptide further comprises at least one or more (such as about at least 10, 15, 20, 25, 30, 35 or all) of residues selected from G96, Q98, R99, E100, K101, G102, K103, C104, L105, D106, P107, S108, L109, K112, S115, V117, G118, G120, E121, D122, T123, P124, Y125, S126, N127, H129, K130, E131, L132, R133, N134, S135, C136, I137, S138, K139, and R140 based upon SEQ ID NO: 1.

In some embodiments, the inhibitory CD93 polypeptide comprises a F238 residue, wherein the amino acid numbering is based on SEQ ID NO 1.

In some embodiments, the inhibitory CD93 polypeptide comprises one, two, three, four or five of the five EGF-like regions of CD93 (such as human CD93) or a variant thereof. In some embodiments, the inhibitory CD93 polypeptide comprises an amino acid sequence of residues C257-M469 or P260-T468 of SEQ ID NO: 1 or variant thereof having at least about 80% (such as about 85% 90%, 91%, 92%, 93% 94%, 95%, 96%, 97%, 98% or 99%) sequence identity to residues C257-M469 or P260-T468 of SEQ ID NO: 1.

In some embodiments, the variant described herein is a natural variant. In some embodiments, the variant does not comprise a non-conservative substitution. In some embodiments, the variant only comprises one or more conservative substitution. In some embodiments, the one or more conservative substitutions comprise or consist of the substitutions shown in Table 1 below under the heading of “Preferred substitutions.”

TABLE 1
Amino acid substitutions

| Original | | |
|---|---|---|
| Residue | Exemplary Substitutions | Preferred Substitutions |
| Ala (A) | Val: Leu: Ile | Val |
| Arg (R) | Lys: Gln: Asn | Lys |
| Asn (N) | Gln: His: Asp: Lys: Arg | Gln |
| Asp (D) | Glu: Asn | Glu |
| Cys (C) | Ser: Ala | Ser |
| Gln (Q) | Asn: Glu | Asn |
| Glu (E) | Asp: Gln | Asp |
| Gly (G) | Ala | Ala |
| His (H) | Asn: Gln: Lys: Arg | Arg |
| Ile (I) | Leu: Val: Met: Ala: Phe: Norleucine | Leu |
| Leu (L) | Norleucine: Ile: Val: Met: Ala: Phe | Ile |
| Lys (K) | Arg: Gln: Asn | Arg |
| Met (M) | Leu: Phe: Ile | Leu |
| Phe (F) | Trp: Leu: Val: Ile: Ala: Tyr | Tyr |
| Pro (P) | Ala | Ala |
| Ser (S) | Thr | Thr |
| Thr (T) | Val: Ser | Ser |
| Trp (W) | Tyr: Phe | Tyr |
| Tyr (Y) | Trp: Phe: Thr: Ser | Phe |
| Val (V) | Ile: Leu: Met: Phe: Ala: Norleucine | Leu |

In some embodiments, the inhibitory CD93 polypeptide binds to IGFBP7 with a greater affinity than for MMNR2. In some embodiments, the inhibitory CD93 polypeptide binds to IGFBP7 with a K_{D} of at most half one-fifth, one-tenth, one-twentieth, one-fiftieth, one-hundredth, one-thousandth of that of the binding between the inhibitory CD93 polypeptide and MMNR2.

In some embodiments, the inhibitory CD93 polypeptide binds to IGFBP7 with a greater affinity than CD93. In some embodiments, the inhibitory CD93 polypeptide binds to IGFBP7 with a K_{D} of at most half one-fifth, one-tenth, one-twentieth, one-fiftieth, one-hundredth, one-thousandth of that of the binding between wildtype CD93 (such as the polypeptide set forth in SEQ ID NO: 1) and IGFBP7.

In some embodiments, the inhibitory CD93 polypeptide further comprises a stabilizing domain. The stabilizing domain can be any domain that stabilizes the inhibitory IGFBP7 polypeptide (for example, extending half-life of the inhibitory IGFBP7 polypeptide in vivo). In some embodiments, the stabilizing domain is an Fc domain. Exemplar Fc domains include those described under “Fc fragment” section.

In some embodiments, the inhibitory polypeptide is about 50 to about 1000 amino acids in length, such as about 50-800, 50-500, 50-400, 50-300 or 50-200 amino acids in length. In some embodiments, the inhibitory polypeptide is about 50 to about 100 amino acids, about 100 to about 150 amino acids, or about 150 amino acids to about 200 amino acids in length.

B. Inhibitory IGFBP Polypeptides

The methods described herein in some embodiments involve use of polypeptides that block the interaction between CD93 and IGFBP7 comprising a variant of IGFBP7 (“inhibitory IGFBP7 polypeptide”). The present application in one aspect provides novel and non-naturally occurring inhibitory IGFBP7 polypeptides described herein.

In some embodiments, the inhibitory IGFBP7 polypeptide binds to CD93 but does not activate CD93.

In some embodiments, the inhibitory IGFBP7 polypeptide binds to CD93 with a greater affinity than for IGF-1, IGF-2, and/or IGF1R. In some embodiments, the inhibitory IGFBP7 polypeptide binds to IGFBP7 with a K_{D} of at most half, one-fifth, one-tenth, one-twentieth, one-fiftieth, one-hundredth, one-thousandth of that of the binding between the inhibitory IGFBP polypeptide and IGF-1, IGF-2, and/or IGF1R.

In some embodiments, the inhibitory IGFBP7 polypeptide binds to CD93 with a greater affinity than IGFBP7. In some embodiments, the inhibitory IGFBP7 polypeptide hinds to CD93 with a K_{D} of at most half, one-fifth, one-tenth, one-twentieth, one-fiftieth, one-hundredth, one-thousandth of that of the binding between the wildtype IGFBP7 (such as the polypeptide set forth in SEQ ID NO:2) and CD93.

In some embodiments, the inhibitory IGFBP7 polypeptide comprises the IB domain of IGFBP7 (such as human IGFBP7) or a variant thereof. In some embodiments, the inhibitory IGFBP7 polypeptide comprises an amino acid sequence of residues S28-G106 of SEQ ID NO: 2 or variant thereof having at least about 80% (such as about 85%, 90%, 91%, 92%, 93%, 94%, 95%, 96%, 97%, 98%, or 99%) sequence identity to residues S28-G106 of SEQ ID NO: 2.

In some embodiments, the inhibitory IGFBP7 polypeptide comprises or further comprises the Kazal-like domain of the IGFBP7 (such as a human IGFBP7) or a variant thereof. In some embodiments, the inhibitory IGFBP7 polypeptide comprises or further comprises an amino acid sequence of residues P105-Q158 of SEQ ID NO:2 or variant thereof having at least about 80% (such as about 85%, 90%, 91%, 92%, 93%, 94%, 95%, 96%, 97%, 98%, or 99%) sequence identity to residues P105-Q158 of SEQ ID NO:2.

In some embodiments, the inhibitory IGFBP7 polypeptide comprises or further comprises the Ig-like C2 domain of the IGFBP7 (such as a human IGFBP7) or a variant thereof. In some embodiments, the inhibitory IGFBP7 polypeptide comprises or further comprises an amino acid sequence of residues P160-T264 of SEQ ID NO:2 or variant thereof having at least about 80% (such as about 85%, 90%, 91%, 92%, 93%, 94%, 95%, 96%, 97%, 98%, or 99%) sequence identity to residues P160-T264 of SEQ ID NO:2.

In some embodiments, the variant described herein is a natural variant. In some embodiments, the variant does not comprise a non-conservative substitution. In some embodiments, the variant only comprises one or more conservative substitution. In some embodiments, the one or more conservative substitutions comprise or consist of the substitutions shown in Table 1 under the heading of “Preferred substitutions.”

In some embodiments, the inhibitory IGFBP7 polypeptide also blocks interaction between CD93 and MMNR2. In some embodiments, the inhibitory IGFBP7 polypeptide binds to the same epitope of CD93 from the epitope that MMNR2 binds to. In some embodiments, the inhibitory IGFBP7 polypeptide binds to a distinct epitope of CD93 from the epitope that MMNR2 binds to.

In some embodiments, the inhibitory IGFBP7 polypeptide does not block the interaction between CD93 and MMNR2.

In some embodiments, the inhibitory IGFBP7 polypeptide is a soluble polypeptide.

In some embodiments, the inhibitory IGFBP7 polypeptide is membrane bound. In some embodiments, the membrane bound inhibitory IGFBP7 polypeptide binds to CD93 but does not trigger, or attenuates CD93/IGFBP7 signaling. In some embodiments, the membrane bound inhibitory IGFBP7 polypeptide is introduced by a gene editing system or an mRNA delivery vehicle.

In some embodiments, the inhibitory IGFBP polypeptide further comprises a stabilizing domain. The stabilizing domain can be any domain that stabilizes the inhibitory IGFBP7 polypeptide (for example, extending half-life of the inhibitory IGFBP7 polypeptide in vivo). In some embodiments, the stabilizing domain is an Fc domain. Exemplary Fc domains include those described under “Fc fragment” section.

In some embodiments, the inhibitory polypeptide is about 50 to about 1000 amino acids in length, such as about 50-800, 50-500, 50-400, 50-300 or 50-200 amino acids in length. In some embodiments, the inhibitory polypeptide is about 50 to about 100 amino acids, about 100 to about 150 amino acids, or about 150 amino acids to about 200 amino acids in length.

Other Agents that Inhibit the IGFBP3/CD93 Signaling Pathway

Other agents that can inhibit the IGFBP3/CD93 other than those described above are also contemplated to be used in methods described herein. In some embodiments, the agent comprises a peptide, a polypeptide, a peptide analog, a fusion peptide an aptamer, an avimer, an anticalin, a speigelmer, or a small molecule compound.

In some embodiments, the agent reduces the expression of CD93 (such as a human CD93). In some embodiments, the agent reduces the expression of CD93 (such as a human CD93) by at least 5%, 10%, 20%, 30%, 40%, 50%, 60%, 70%, 80%, 90%, or 95% as compared to the level of CD93 without the agent. In some embodiments, the agent renders the expression of CD93 comparable as a reference level. In some embodiments, the reference level is the level of CD93 expression in a non-tumor organ in the subject. In some embodiments, the reference level is the level (or average level) of CD93 expression in a subject or group of subjects that do not have the disease or condition or abnormal vascular structure.

In some embodiments, the agent reduces the expression of IGFBP7 (such as a human IGFBP7). In some embodiments, the agent reduces the expression of IGFBP7 (such as a human IGFBP7) by at least 5%, 10%, 20%, 30%, 40%, 50%, 60%, 70%, 80%, 90%, or 95% as compared to the level of IGFBP7 without the agent. In some embodiments, the agent renders the expression of IGFBP7 comparable as a reference level. In some embodiments, the reference level is the level of IGFBP7 expression in a non-tumor organ in the subject. In some embodiments, the reference level is the level (or average level) of IGFBP7 expression in a subject or group of subjects that do not have the disease or condition or abnormal vascular structure.

In some embodiments, the agent comprises a siRNA, a shRNA, a miRNA, or an antisense RNA that targets CD93 (such as a human CD93). In some embodiments, the siRNA, shRNA miRNA or antisense RNA that specifically targets IGFBP7 (such as a human IGFBP7).

In some embodiments, the agent comprises a genome-editing system that targets CD93 or IGFBP7. In some embodiments, the genome-editing system comprises a DNA nuclease such as an engineered (e.g., programmable or targetable) DNA nuclease to induce genome editing of a target DNA sequence of CD93 or IGFBP7. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, the genome editing comprises modifying CD93 so that the modified CD93 no longer binds to IGFBP7 or binds to IGFBP7 to a less extent than wildtype CD93. In some embodiments, the modification comprises inserting a transgene comprising a variant of CD93. In some embodiments, the variant CD93 has a mutation at F238 based upon SEQ ID NO: 1. In some embodiments, the variant CD93 has a F238T mutation based upon SEQ ID NO: 1.

In some embodiments, the gnome editing comprises modifying IGFBP7 so that the modified IGFBP7 no longer binds to CD93 or binds to CD93 to a lesser extent than wildtype IGFBP7. In some embodiments, the modification comprises inserting a transgene comprising a variant of IGFBP7. In some embodiments, the variant of IGFBP7 has a c-type lectin domain, and the c-type lection domain of IGFBP7 is not derived from IGFBP7.

### Vascular Maturation/Normalization

The successful functioning of all tissues depends on the establishment of a hierarchically structured, mature vascular network. In contrast to the healthy state, a number of human diseases show a dysregulated excess of new blood vessel formation. Solid tumors are one characterized example. Much more than a mass of proliferating cancer cells, a solid tumor is an assembly of cancer cells, a blood vessel network, lymphatic vessels, and a variety of other cells all of which contribute to the local microenvironment. Angiogenesis within solid tumors is driven largely by hypoxia. This hypoxia, a hallmark of the tumor microenvironment, leads directly to the production of proangiogenic factors such as VEGF via modulation of oxygen sensing molecules. See Goel et al., Cold Spring Harb Perspect Med 2012:2:a006486.

The microenvironmental abundance of VEGF and other proangiogenic factors drives continual angiogenesis and the production of an abnormal blood vessel network. Structurally, vessels are often dilated, weave a tortuous path, and show heterogeneity of distribution such that certain areas within a tumor are hypovascular and others hypervascular. At the cellular level, proangiogenic factors induce weakening of VE-Cadherin-mediated endothelial cell (EC) junctions and EC migration, altering vessel wall architecture. Similarly, the perivascular cells (PVCs, comprised of pericytes and vascular smooth muscle cells (VSMCs)) are often only loosely attached to ECs and are reduced in number. Finally, the perivascular basement membrane (BM) is also structurally abnormal in tumors-excessively thin or absent in certain regions and abnormally thick in others. See Goel et al., Cold Spring Harb Perspect Med 2012:2:a006486.

A direct consequence of these structural derangements is marked aberration of tumor vascular function. The haphazard and bizarre distribution of vessels leads to heterogeneous blood flow, sluggish in some regions and excessive in others. In addition, reduced PVC coverage, EC dissociation, and an excess of vesiculo-vaculor organelles (VVOs) results in marked tumor vessel permeability, with excess extravasation of fluid and protein into the extracellular compartment. This leakiness, together with a relative absence of functional intratumoral lymphatic vessels, leads to a marked increase in the tumor interstitial fluid pressure (IFP) to a level that equilibrates with intravascular pressure, which results in reduced transvascular flow. Furthermore, the compressive forces applied by the proliferating mass of cancer cells can cause vascular compression and collapse. The net result is a heterogeneous blood supply, and resultant hypoxia and acidosis. The physiological changes described have a direct effect on solid tumor behavior, hypoxic tumor cells often show a more aggressive phenotype, activating oncogenes and passing through an “epithelial to mesenchymal transition” (EMT), which heightens their metastatic potential. Moreover, the hostile microenvironment impairs the function of antitumor immune cells, the delivery of which into the tumor is also impaired. Importantly, tumor response to therapy is also impacted. Hypoxia is known to reduce tumor cell sensitivity to radiation and chemotherapy, and the delivery of systeIGFBP7ly administered cytotoxics into tumors is dramatically impeded, especially in areas of low blood flow and raised tumor IFP. See Goel et al., Cold Spring Harb Perspect Med 2012:2:a006486.

The present application provides methods and compositions that are useful in normalizing vascular (i.e., promoting maturation of the abnormal vasculature) in diseases or conditions (such as cancer, such as solid tumor). In some embodiments, the abnormal vascular is associated with hypoxia.

“Normalization of vasculature,” “normalizing immature and leaky blood vessel,” “vascular maturation.” or “promoting the formation of a functional vascular network.” and “promoting a favorable tumor microenvironment” generally refer to or comprises conversion of a network of leaky, tortuous, disorganized vessels (e.g., tumor vessels) to a more organized network of vessels that are less permeable, less dilated and/or less tortuous. In some embodiments, vascular normalization is characterized by more mature vessels (e.g., longer vessels, circular vessels). In some embodiments, vascular normalization is characterized by increased association of pericytes and/or smooth muscle cells with the endothelial cells lining the walls of the vessels, formation of a more normal basement membrane (e.g., having a more physiological thickness) and/or closer association of vessels with the basement membrane. Normalization of vasculature can also involve pruning of immature vessels, along with increased integrity and stability of the remaining vasculature. In some embodiments, the normalization of vascular described herein is characterized by maintenance of vessel density.

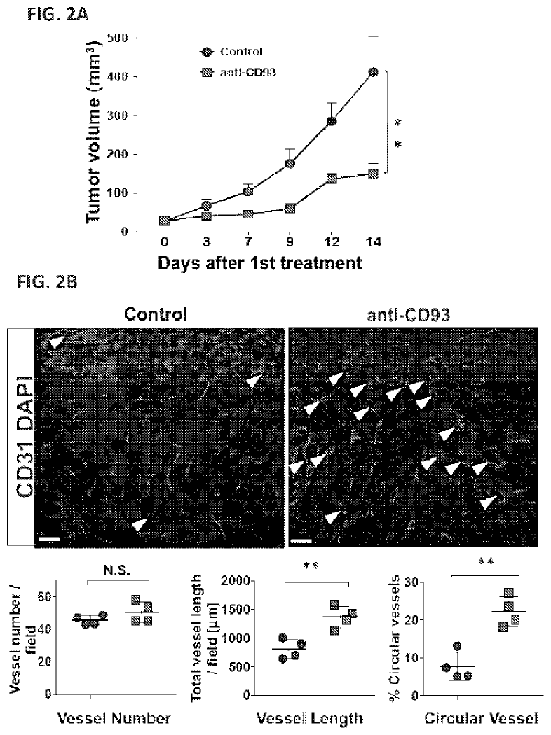

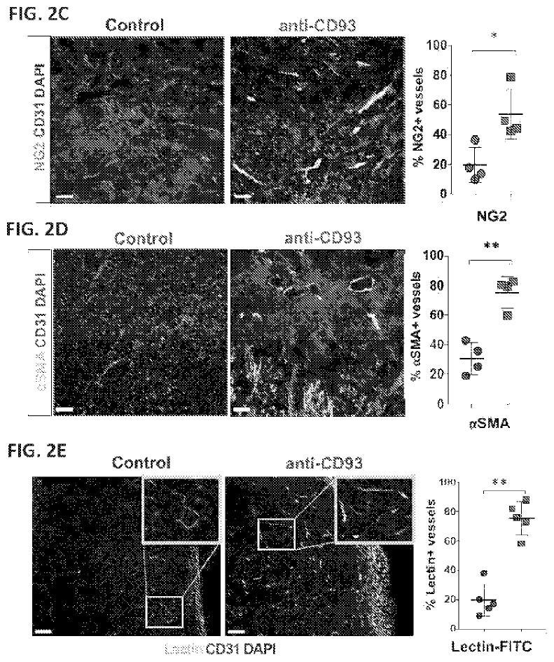

In some embodiments, matureness of vessels (or vascular normalization) can be characterised by the morphology of vessels. In some embodiments, the vascular normalization is characterized by an increase of length of the vessels in the tissue. The length of vessels can be measured in the unit of total vessel length per field (e.g., μm) as described in Examples (see for example, FIG. 2B). In some embodiments, the length of vessels (e.g., the total length per field) is increased by at least about 10%, 20%, 30% 40%, 50%, 60%, 70%, 80%, 90%, or 100% post administration of the IGFBP7/CD93 blocking agent. In some embodiments, the vessels are identified by CD31 expression.

In some embodiments, the vascular normalization is characterized by an increase of circular vessel percentage (% of circular vessel/total vessel) in the tissue. Circular vessel percentage can be measured by dividing circular vessel numbers by total vessels such as described in Examples (see for example, FIG. 2B). In some embodiments, the circular vessel percentage is increased by at least about 10%, 20%, 30%, 40%, 50%, 60%, 70%, 80% 90%, or 100% post administration of the IGFBP7/CD93 blocking agent In some embodiments, the vessels are identified by CD31 expression.

In some embodiments, the vascular normalization is characterized by a maintenance of vessel density of the vessels in the tissue. The density of vessels can be measured in the unit of vessel number per field as described in Examples (see for example, FIG. 2B). In some embodiments, vessel density is not decreased by more than about 30%, 20%, 10%, or 5% post administration of the IGFBP7/CD93 blocking agent. In some embodiments, vessel density is not increased by more than about 30%, 20%, 10%, or 5% post administration of the IGFBP7/CD93 blocking agent. In some embodiments, vessel density is neither increased, nor decreased by more than about 30%, 20%, 10%, or 5% post administration of the IGFBP7/CD93 blocking agent. In some embodiments, the vessels are identified by CD31 expression.

In some embodiments, matureness of vessels (or vascular normalization) can be characterized by a denser let el of pericytes (e.g., NG2 pericytes) and/or a denser level of smooth muscle cells (e.g., α-SMA− smooth muscle cells). In some embodiments, the vascular normalization is characterized by an increase of NG2 expression on vessels. In some embodiments, the NG2 expression on vessels is increased by at least about 25%, 50%, 75%, 100%, 125%, 150%, 175%, or 200% post administration of the IGFBP7/CD93 blocking agent. In some embodiments, the vascular normalization is characterized by an increase of α-SMA− expression on vessels. In some embodiments, the α-SMA+ expression on vessels is increased by at least about 25%, 50%, 75%, 100%, 125%, 150%, 175%, 200% 225%, or 250% post administration of the IGFBP7/CD93 blocking agent. In some embodiments, the vascular normalisation is characterized by an increase of ICAM expression on vessels. In some embodiments, the ICAM− expression on vessels is increased by at least about 10%, 20%, 30%, 40%, 50%, 60%, or 70% post administration of the IGFBP7/CD93 blocking agent. In some embodiments, the vascular normalization is characterized by a decrease of activated integrin β1 expression on vessels. In some embodiments, the activated integrin β1 expression on vessels is decreased by at least about 10%, 20%, 30%, 40%, or 50% post administration of the IGFBP7/CD93 blocking agent. In some embodiments, the vessels are identified CD31 expression.

In some embodiments, matureness of vessels (or vascular normalization) can be characterized by the vascular perfusion and/or permeability. In some embodiments, the vascular normalization is characterized by an increased vascular permeability or perfusion. Permeability or perfusion can be assessed, for example, as described in Examples (e.g., FIG. 2E) by assessing if the distribution of administered drug (such as lectin) in vessels. In some embodiments, the vascular perfusion is increased by at least about 25%, 30%, 75%, 100%, 125%, 150%, 175%, 200%, 225%, 250%, 275%, or 300% post administration of the IGFBP7/CD93 blocking agent.

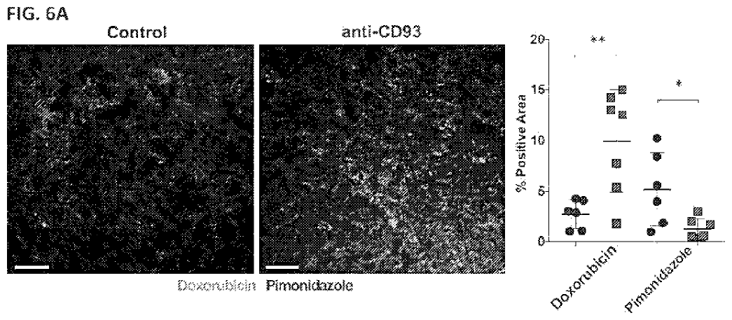

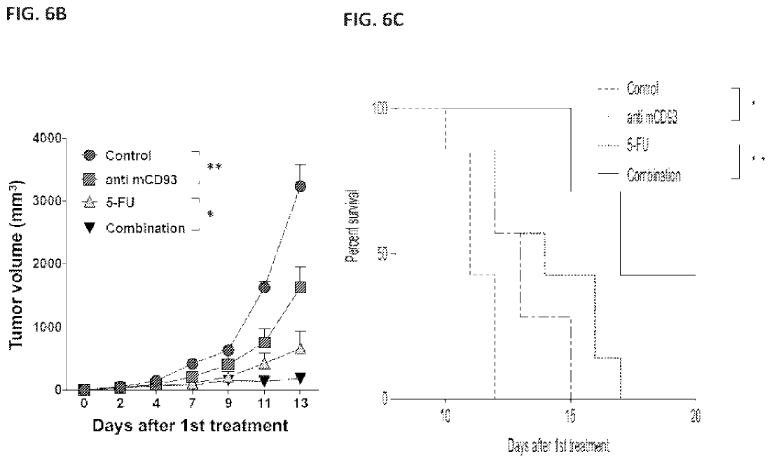

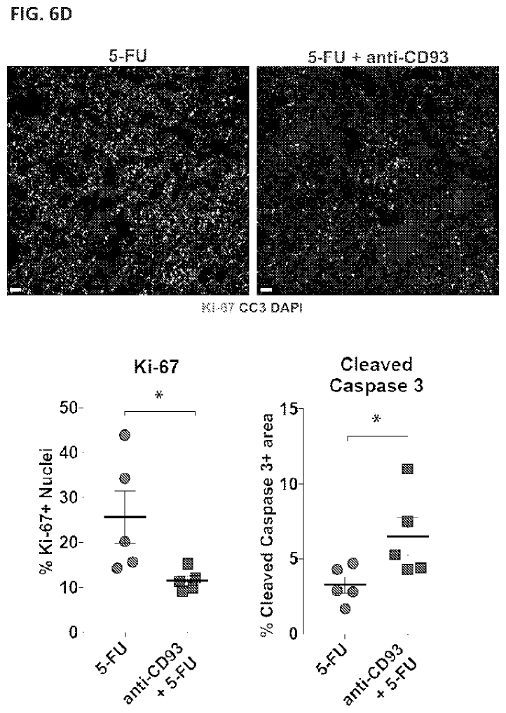

In some embodiments, the vascular normalization is characterized by decreased hypoxia in the tissue. Tumor hypoxia can be assessed, for example, as described in the Examples (such as FIG. 6A). In some embodiments, the tumor hypoxia is assessed by a pimonidazole positive percentage (i.e., pimonidazole positive area divided by total tumor area). In some embodiments, the tumor hypoxia is decreased by at least about 10%, 20%, 30%, 40%, 50%, 60%, 70%, 80%, or 90% post administration of the IGFBP7/CD93 blocking agent.

In some embodiments, the vascular normalization is characterized by a more effective drug delivery. Effectiveness of drug delivery can be determined, for example, by assessing the distribution of drug in the tissue (such as tumor tissue) post drug delivery (e.g., as described in the Examples (e.g., FIG. 6A)). In some embodiments, the presence/distribution of a drug (such as a chemotherapeutic drug) in the tissue after delivery is increased by at least about 25%, 50%, 75%, 100%, 125%, 150%, 175%, 200%, 225%, 250%, 275%, or 300% post administration of the IGFBP7/CD93 blocking agent.

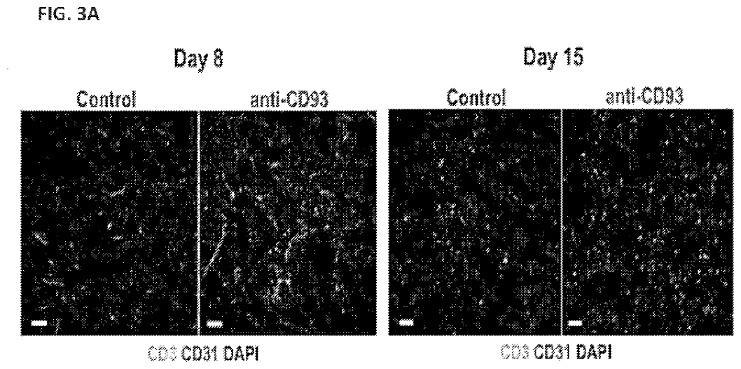

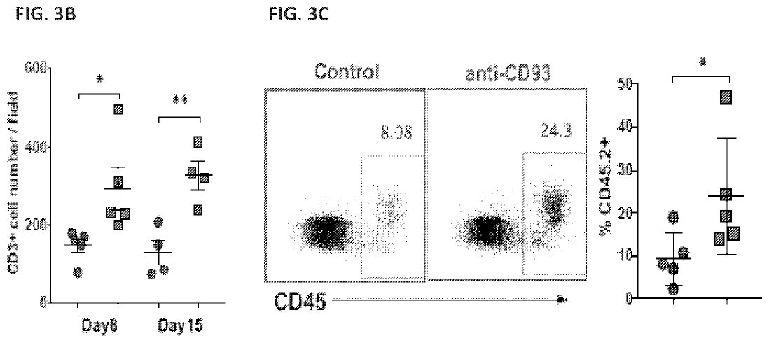

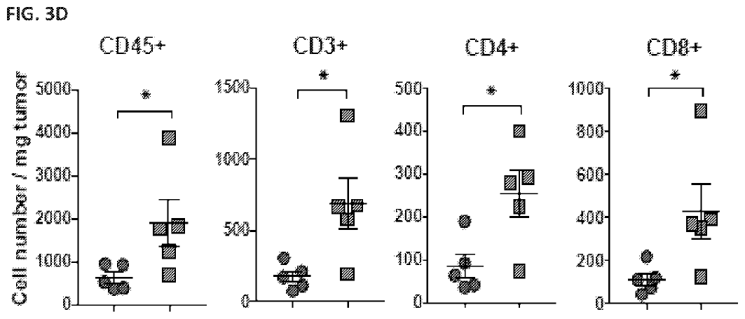

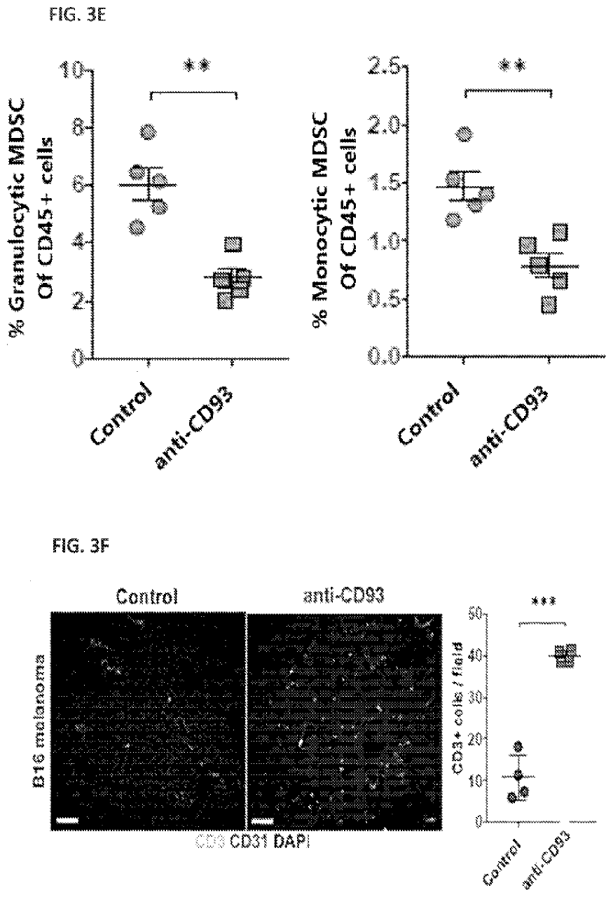

In some embodiments, the vascular normalization is characterized by an increased infiltration of immune cells in the tissue (e.g., tumor tissue) The infiltration of immune cells in the tissue can be measured by assessing the percentage of immune cells in the tissue (e.g., tumor tissue) (e.g., by measuring the number of immune cells in the tissue divided by a tumor eight unit (e.g., mg) or by measuring the numbering of immune cells in the tissue divided by a field unit as described in FIGS. 3A and 3D). In some embodiments, the immune cells are tumor-infiltrating lymphocytes. In some embodiments, the immune cells comprise CD45− leukocytes. In some embodiments, the immune cells comprise CD3− T cells. In some embodiments, the immune cells comprise CD4− cells. In some embodiments, the immune cells comprise CD8+ T cells. In some embodiments, the immune cells are endogenous immune cells. In some embodiments, the immune cells are exogenous immune cells. In some embodiments, the immune cells are engineered immune cells derived from the subject (for example, CAR T cells). In some embodiments, the percentage of immune, cells in the tissue (e.g., tumor tissue) is increased by at least about 25%, 50%, 75%, 100%, 125%, 150%, 175%, 200%, 225%, 250%, 275%, or 300% post administration of the IGFBP7/CD93 blocking agent.

In some embodiments, the ratio of suppressor immune cells in the infiltrated immune cells are decreased post administration of the IGFBP7/CD93 blocking agent. In some embodiments, the suppressor immune cells comprise myeloid-derived suppressor cells (MDSC). In some embodiments, the MDSC comprise granulocytic MDSCs (e.g., CD3− CD11c-CD11b+Ly6G−Ly6C−CD45+ leukocytes). In some embodiments, the MDSC comprise monocytic MDSCs (e.g. CD3−CD11c−CD11b+Ly6G−Ly6C+CD45+ leukocytes). In some embodiments, the MDSC comprise both granulocytic MDSCs and monocytic MDSCs. In some embodiments, the ratio of the suppressor immune cells in the infiltrated immune cells is decreased by at least 10%, 20%, 30%, 40%, or 50% post administration of the IGFBP7/CD93 blocking agent.

The different parameters described in the above section (such as vessel length, morphology, hypoxia, perfusion, infiltration of immune cells, drug delivery) can be assessed at different time points post one or more administration of the IGFBP7CD93 blocking agent. In some embodiments, the parameter is assessed after 14 days of administration of the IGFBP7/CD93 blocking agent, wherein the agent is administered at a frequency of about twice a week for two weeks.

### Endpoints

Any parameters described in the “Vascular maturation normalization” section (such as vessel length, morphology, hypoxia, perfusion, infiltration of immune cells, drug delivery) can be used as a characteristic of the methods described above (such as methods of treating a cancer). The “Vascular maturation/normalization” section is incorporated here in its entirely for the discussion of features of Various embodiments of the methods described above.

In some embodiments, the subject has a decreased proliferation of tumor cells and/or an increased apoptosis of tumor cells. Proliferation and apoptosis of tumor cells can be assessed by a proliferation marker or apoptotic marker (such as Ki-67 and cleaved caspase 3 (CC3) as described in the Examples). In some embodiments, the proliferation of tumor cells is characterized by Ki-67-positive cells in the tumor. In some embodiments, the Ki-67 positive cells in the tumor is decreased by at least about 10%, 20%, 30%, 40%, 50%, or 60% post administration of the IGFBP7/CD93 blocking agent. In some embodiments, the apoptosis of tumor cells is characterized by CC3-positive cells in the tumor tissue. In some embodiments, the CD3-positive cells in tumor tissue is increased by at least about 10%, 20%, 30%, 40%, 50%, 60%, 70%, 80%, or 90% post administration of the IGFBP7/CD93 blocking agent.

In some embodiments, the subject has a decrease of the size of a tumor, decrease of the number of cancer cells, or decrease of the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

### Disease or Condition

The methods described herein are applicable to any disease or conditions associated with an abnormal vascular structure. In some embodiments, the disease or condition is an age-related macular degeneration (ARMD). In some embodiments, the disease or condition is a cutaneous psoriasis. In some embodiments, the disease or condition is a benign tumor. In some embodiments, the disease or condition is a cancer.

### Cancer

In some embodiments, the disease or condition described herein is a cancer. Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the cancer is a solid tumor.

In some embodiments, the cancer comprises CD93− tumor endothelial cells. In some embodiments, at least 10%, 20%, 30%, 40%, 50%, 60%, 70%, 80%, or 90% of the endothelial cells in the tumor are CD93 positive. In some embodiments, the cancer comprises at least 20%, 40%, 60%, 80% or 100% more CD93− endothelial cells than that of a normal tissue in the subject. In some embodiments, the cancer comprises at least 20%, 40%, 60%, 80%, or 100% more CD3+ endothelial cells than that of a corresponding organ in a subject or a group of subjects who do not have the cancer.

In some embodiments, the cancer comprises IGFBP7− blood vessels. In some embodiments, the cancer comprises at least 20%, 40%, 60%, 80%, or 100% more IGFBP7− blood vessels than that of a normal tissue in the subject. In some embodiments, the cancer comprises at least 20%, 40%, 60%, 80%, or 100% more IGFBP7+ blood vessels than that of a corresponding organ in a subject or a group of subjects who do not have the cancer.

In some embodiments, the cancer (e.g., a solid tumor) is characterized by tumor hypoxia. Tumor hypoxia can be assessed, for example, as described in the Examples (such as FIG. 6A). In some embodiments, the cancer is characterized by a pimonidazole positive percentage (i.e., pimonidazole positive area divided by total tumor area) of at least about 1%, 2%, 3%, 4%, or 5%.

Examples of cancers that may be treated by the methods of this application include, but are not limited to, anal cancer, astrocytoma (e.g., cerebellar and cerebral), basal cell carcinoma, bladder cancer, hone cancer (e.g., osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer (e.g., TNBC), cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy. 19th Edition. § on Hematology and Oncology, published by Merck Sharp &, Dohme Corp., 2011 (ISBN 978-0-911910-19-3); The Merck Manual of Diagnosis and Therapy, 20th Edition. § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer is triple-negative breast cancer (TNBC, for example TNBC with high IGFBP or CD93 expression). In some embodiments, the cancer is melanoma. In some embodiments, the patient is resistant to a prior therapy comprising administration of an immune checkpoint inhibitor, e.g., an anti-PD1 antibody, an anti-PD-L1 antibody, an anti-CTLA4 antibody, or a combination thereof.

### Subject

In some embodiments, the subject is a mammal (such as a human).

In some embodiments, the subject has a tissue comprising abnormal vascular comprising CD93, endothelial cells. In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93− endothelial cells than that of a normal tissue in the subject. In some embodiments, the tissue with abnormal vascular comprises at least 20%, 40%, 60%, 80%, or 100% more CD93+ endothelial cells than that of a corresponding organ in a subject or a group of subjects who do not hay e the abnormal vascular.

In some embodiments, the subject has a tissue comprising abnormal vascular comprising IGFBP7− blood vessels. In some embodiments, the tissue comprises at least 20%, 40%, 60%, 80%, or 100% more IGFBP71 blood vessels than that of a normal tissue in the subject. In some embodiments, the tissue comprises at least 20%, 40%, 60%, 80% or 100% more IGFBP7− blood vessels than that of a corresponding organ in a subject or a group of subjects who do not hay e the abnormal vascular.

In some embodiments, the subject is selected for treatment based upon an abnormal vascular structure. In some embodiments, the abnormal vascular structure is characterized by CD93+ endothelial cells (for example, by measuring CD93+ CD31− cells). In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93+ endothelial cells than that of a normal tissue in the subject. In some embodiments, the tissue with abnormal vascular comprises at least 20%, 40%, 60% 80%, or 100% more CD93+ endothelial cells than that of a corresponding organ in a subject or a group of subjects who do not have the abnormal vascular.

In some embodiments, the abnormal vascular structure is characterized by an abnormal level of IGFBP7+ blood vessels. In some embodiments, the tissue comprises at least 20%, 40%, 60%, 80%, or 100% more IGFBP7+ blood vessels than that of a normal tissue in the subject. In some embodiments, the tissue comprises at least 20%, 40%, 60% 80%, or 100% more IGFBP7− blood vessels than that of a corresponding organ in a subject or a group of subjects who do not have the abnormal vascular.

In some embodiments, the subject has at least one prior therapy. In some embodiments, the prior therapy comprises a radiation therapy, a chemotherapy and/or an immunotherapy. In some embodiments, the subject is resistant, refractory, or recurrent to the prior therapy. In some embodiments, the prior therapy comprises administration of an immune checkpoint inhibitor, e.g., an anti-PD1 antibody, an anti-PD-L1 antibody, an anti-CTLA4 antibody, or a combination thereof.

### Combination Therapy

The present application also provides methods administering an agent that inhibits the IGFBP7/CD93 signaling pathway as described herein (“the IGFBP7/CD93 blocking agent”) into a subject for treating a disease or condition (such as cancer), wherein the method further comprises administering a second agent or therapy. In some embodiments, the second agent or therapy is a standard or commonly used agent or therapy for treating the disease or condition. In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a cell therapy (such as a cell therapy comprising an immune cell (e.g., CAR T cell)). In some embodiments, the second agent or therapy comprises an angiogenesis inhibitor.

In some embodiments, the second agent is a chemotherapeutic agent. In some embodiments, the second agent is antimetabolite agent. In some embodiments, the antimetabolite agent is 5-FU.

In some embodiments, the second agent is an immune checkpoint modulator. In some embodiments, the immune checkpoint modulator is an inhibitor of an immune checkpoint protein selected from the group consisting of PD-L1, PD-L2, CTLA4, PD-L2, PD-1, CD47, TIGIT, GITR, TIM3, LAG3, CD27, 4-1BB, and B7H4. In some embodiments, the immune checkpoint protein is PD-1. In some embodiments, the second agent is an anti-PD-1 antibody or fragment thereof. In some embodiments, the second agent is an anti-CTLA4 antibody or fragment thereof. In some embodiments, the second agent is a combination of an anti-PD1 antibody or fragment thereof and an anti-CTLA4 antibody or fragment thereof.

In some embodiments, the IGFBP7/CD93 blocking agent administered simultaneously with the second agent or therapy. In some embodiments, the IGFBP7/CD93 blocking agent that inhibits the IGFBP7/CD93 signaling pathway is administered concurrently with the second agent or therapy. In some embodiments, the IGFBP7/CD93 blocking agent is administered sequentially with the second agent or therapy. In some embodiments, the IGFBP7/CD93 blocking agent is administered in the same unit dosage form as the second agent or therapy. In some embodiment, the IGFBP7/CD93 blocking agent is administered in a different unit dosage form from the second agent or therapy.

### Dosing Regimen and Routes of Administration

The dose of the IGFBP7/CD93 blocking agent and, in some embodiments, the second agent as described herein, administered to a subject (such as a human) may vary with the particular composition, the method of administration, and the particular kind and stage of disease or condition (such as a cancer) being treated. The amount should be sufficient to produce a desirable response, such as a therapeutic response against the disease or condition (such as a cancer). In some embodiments, the amount of the IGFBP7/CD93 blocking agent and/or the second agent is a therapeutically effective amount.

In some embodiments, the amount of the IGFBP7/CD93 blocking agent is an amount sufficient to promote normalization of vessels (such as increasing the length of vessels, increasing the number of circular vessels, maintaining the density of vessels, and/or increasing the pericytes and/or smooth muscle cells), an increase in the perfusion of tissue (such as tumor tissue), a decrease in hypoxia, an increase in the amount of drug delivered into the tissue, an increase in immune cell infiltration in the tissue, and/or inhibition of tumor cell growth.

In some embodiments, the amount of the IGFBP7/CD93 blocking agent is an amount sufficient to produce an increase in the length of the vessels in the tissue (e.g., the total length per field) by at least about 10%, 20%, 30%, 40%, 50%, 60%, 70%, 80%, 90%, or 100% post administration of the IGFBP7/CD93 blocking agent. In some embodiments, the amount of the IGFBP7/CD93 blocking agent is an amount sufficient to produce an increase in the circular vessel percentage (% of circular vessel total vessels) in the tissue by at least about 10%, 20%, 30%, 40%, 50%, 60%, 70%, 80%, 90% or 100% post administration of the IGFBP7/CD93 blocking agent. In some embodiments, the amount of the IGFBP7/CD93 blocking agent is an amount sufficient to maintain the density of vessels in the tissue post administration of the IGFBP7/CD93 blocking agent.

In some embodiments, the amount of the IGFBP7/CD93 blocking agent is an amount sufficient to produce an increase in pericytes in the tissue (e.g., NG2 positive expression on vessels) by at least about 25%, 50%, 75%, 100%, 125%, 150%, 175%, or 200% post administration of the IGFBP7/CD93 blocking agent. In some embodiments, the amount of the IGFBP7/CD93 blocking agent is an amount sufficient to produce an increase in smooth muscle cells in the tissue (e.g., α-SMA expression on vessels) by at least about 25%, 30%, 75%, 100%, 125%, 150%, 175%, 200%, 225%, or 250% post administration of the IGFBP7/CD93 blocking agent. In some embodiments, the amount of the IGFBP7/CD93 blocking agent is an amount sufficient to produce an increase in ICAM+ expression by at least about 10%, 20%, 30%, 40%, 50%, 60%, or 70% post administration of the IGFBP7/CD93 blocking agent. In some embodiments, the amount of IGFBP7/CD93 blocking agent is an amount sufficient to produce a decrease in the activated integrin β1 expression by at least about 10%, 20%, 30%, 40%, or 50% 6 post administration of the IGFBP7% CD93 blocking agent.

In some embodiments, the amount of the IGFBP7/CD93 blocking agent is an amount sufficient to produce an increase in the vascular permeability or perfusion in the tissue by at least about 25%, 50%, 75%, 100%, 125%, 150%, 175%, 200%, 225%, 250%, 275%, or 300% post administration of the IGFBP7/CD93 blocking agent.

In some embodiments, the amount of the IGFBP7/CD93 blocking agent is an amount sufficient to produce a decrease of hypoxia in the tissue by at least about 10%, 20%, 30%, 40%%, 50%, 60%, 70%, 80%, or 90% post administration of the IGFBP7/CD93 blocking agent.

In some embodiments, the amount of the IGFBP7/CD93 blocking agent is an amount sufficient to produce an increase in the presence/distribution of a drug (such as a chemotherapeutic drug) in the tissue after delivery by at least about 25%, 50%, 75%, 100%, 125%, 150%, 175%, 200%, 225%, 250%, 275%, or 300% post administration of the IGFBP7/CD93 blocking agent.

In some embodiments, the amount of the IGFBP7/CD93 blocking agent is an amount sufficient to produce an increase in the infiltration of immune cells (such as the percentage of immune cells in the tissue) in the tissue by at least about 25%, 50%, 75%, 100%, 125%, 150%, 175%, 200%, 225%, 250%, 275%, or 300% post administration of the IGFBP7/CD93 blocking agent. In some embodiments, the amount of the IGFBP7/CD93 blocking agent is an amount sufficient to produce a decrease in the ratio of the suppressor immune cells in the infiltrated immune cells in the tissue by at least about 10%, 20%, 30% 40%, or 50% post administration of the IGFBP7/CD93 blocking agent.

In some embodiments, the amount of the IGFBP7/CD93 blocking agent is an amount sufficient to produce a decrease in proliferation of cells (e.g., tumor cells) in the tissue by at least about 10%, 20%, 30%, 40%, 50%, or 60% post administration of the IGFBP7/CD93 blocking agent. In some embodiments, the amount of the IGFBP7. C. D93 blocking agent is an amount sufficient to produce an increase in apoptosis of cells (e.g., tumor cells) in the tissue by at least about 10%, 20%, 30%, 40%, 50%, 60%, 70%, 80% or 90% post administration of the IGFBP7/CD93 blocking agent.

In some embodiments, the amount of the IGFBP7/CD93 blocking agent is an amount sufficient to produce a decrease of the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor sin, number of cancer cells, or tumor growth rate in the same subject prior to treatment or compared to the corresponding activity in other subjects not receiving the treatment.

In some embodiments, the IGFBP7/CD93 blocking agent comprises an anti-CD93 antibody. In some embodiments, the subject is a human, and the amount of anti-CD93 antibody for each administration is equivalent to a dose of about 300 μg for a mouse. In some embodiments, the subject is a human, and the amount of anti-CD93 antibody for each administration is no more than about 2 g (such as about 50-75 mg). In some embodiments, the subject is a human, and the amount of anti-CD93 antibody for each administration is no more than about 30 mg/kg (such as about 0.8 mg/kg to about 1.2 mg/kg). In some embodiments, the subject is a human, and the amount of anti-CD93 antibody for each administration 30-45 mg/m². In some embodiments, the subject is a human, and the amount of anti-CD93 antibody for each administration is no more than about 75 mg (or about 1.25 mg/kg, or about 45 mg/m²).

In some embodiments, the IGFBP7/CD93 blocking agent comprises an anti-IGFBP7 antibody. In some embodiments, the subject is a human, and the amount of anti-IGFBP7 antibody for each administration is equivalent to a dose of about 300 μg for a mouse. In some embodiments, the subject is a human, and the amount of anti-IGFBP7 antibody for each administration is no more than about 2 g (such as about 50-75 mg). In some embodiments, the subject is a human, and the amount of anti-IGFBP7 antibody for each administration is no more than about 30 mg/kg (such as about 0.8 mg/kg to about 1.2 mg/kg). In some embodiments, the subject is a human, and the amount of anti-IGFBP7 antibody for each administration 30-45 mg/m². In some embodiments, the subject is a human, and the amount of anti-IGFBP7 antibody for each administration is no more than about 75 mg (or about 1.25 mg/kg, or about 45 mg/m²).

In some embodiments, the anti-IGFBP7 antibody or anti-CD93 antibody is administered for a period of at least about 1, 3, 7, 10, 12, or 14 days. In some embodiments, the anti-IGFBP7 antibody or anti-CD93 antibody is administered at a frequency of at least about twice a week.

In some embodiments, the methods comprise administering a second agent, wherein the second agent is 5-FU. In some embodiments, the subject is a human, and the amount of 5-FU antibody for each administration is equivalent to a dose of about 3 mg to about 4 mg for a mouse.

In some embodiments according to any one of the methods described herein, the IGFBP7/CD93 blocking agent and/or the second agent composition is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the IGFBP7/CD93 blocking agent and/or the second agent is administered intravenously.

### III. Methods of Diagnosis and Prognosis

Provided herein also include methods of diagnosing or prognosing a subject, including, determining the suitability of a subject for the treatment as described in section II or a different therapy, determining the likelihood of responsiveness of a subject to the methods as described in section II or a different therapy, and determining the matureness status of vascular in a tissue in a subject.

In some embodiments, there is provided a method of determining the suitability of a subject for a treatment, comprising measuring levels of CD93 expression in a tissue of a subject. In some embodiments, there is provided a method of determining the suitability of a subject for a treatment, comprising measuring levels of IGFBP7 expression in a tissue of a subject. In some embodiments, the subject has a cancer, and the tissue is a tumor tissue. In some embodiments, the treatment comprises a CD93/IGFBP7 blocking agent. In some embodiments, the treatment comprises a cancer therapy (such as a cell therapy, such as a chemotherapeutic agent). In some embodiments, a higher CD93 or IGFBP7 expression level as compared to a reference level indicates a lower suitability for the treatment.

In some embodiments, there is provided a method of prognosis in a subject having cancer (such as a solid tumor), comprising measuring levels of CD93 expression in a tumor sample in vitro or in vivo, wherein a higher CD93 expression level as compared to a reference level indicates a higher possibility of not responding or responding poorly to a therapy. In some embodiments, the reference level is a level of CD93 expression (such as an average CD93 expression) in a non-tumor sample in the subject or a corresponding tissue in a different subject (or a group of subjects) who does not have cancer.

In some embodiments, there is provided a method of prognosis in a subject having cancer (such as a solid tumor), comprising measuring levels of IGFBP7 expression in a tumor sample in vitro or in vivo, wherein a higher IGFBP7 expression level as compared to a reference level indicates a higher possibility of not responding or responding poorly to a therapy. In some embodiments, the reference level is a level of IGFBP7 expression (such as an average IGFBP7 expression) in a non-tumor sample in the subject or a corresponding tissue in a different subject (or a group of subjects) who does not have cancer.

In some embodiments, the therapy comprises a cell therapy. In some embodiments, the therapy comprises an agent selected from a chemotherapeutic agent (such as antimetabolite agent, such as an immune checkpoint modulator), a radiation agent, or an immunotherapeutic agent. In some embodiments, the agent has a size of no more than 1 μm, 0.5 μm, 0.2 μm, or 0.1 μm.

In some embodiments, there is provided a method of determining matureness status of vascular in a tissue (such as a cancer tissue) in a subject comprising administering an imaging agent comprising an anti-CD93 antibody labeled with an imaging molecule. In some embodiments, the imaging molecule is a radionuclide.

In some embodiments, there is provided a method of determining matureness status of vascular in a tissue (such as a cancer tissue) in a subject comprising administering an imaging agent comprising an anti-IGFBP7 antibody labeled with an imaging molecule. In some embodiments, the imaging molecule is a radionuclide.

### IV. Methods of Identifying Agents that Disrupt Interaction Between CD93 and IGFBP7

The agents described herein can be identified by assessing the ability of the agent to disrupt the interaction between CD93 and IGFBP7. Provided herein are methods of identifying agents (such as antibodies, peptides, polypeptides, peptide analogs, fusion peptides, aptamers, an avimer, an anticalin, a speigelmer, and small molecule compounds) that are useful for treating cancer or one or more aspects of cancer treatment, including, but not limited to: blocking abnormal tumor vascular angiogenesis, normalizing immature and leaks tumor blood vessel, promoting functional vascular network in a tumor, promoting vascular maturation, promoting a favorable tumor microenvironment, increasing immune cell infiltration in a tumor, increasing tumor perfusion, reducing hyperplasia in a tumor, sensitizing tumor to a second therapy, and facilitating delivery of a second agent. The methods generally involve determining whether the candidate agent specifically disrupts the CD93/IGFBP7 interaction, wherein the candidate agent is useful for treating cancer and aspects of cancer treatment if it is shown to specifically disrupt the CD93/IGFBP7 interaction.

The agent can be an antibody, an antibody-like scaffold, a small molecule, fusion protein, peptide, mimetic, or inhibitory nucleotide (e.g., RNAi) directed against (i) CD93, (ii) IGFBP7; (iii) a novel site (e.g., a newly created epitopic determinant) created by the CD93/IGFBP7 interaction, or (iv) a protein complex comprising any of the same.

Thus, for example, in some embodiments, there is provided a method of determining whether a candidate agent is useful for treating cancer, comprising: determining whether the candidate agent specifically disrupts the CD93/IGFBP7 interaction, wherein the candidate agent is useful for treating cancer if it is shown to specifically disrupt the CD93/IGFRP interaction. In some embodiments, the method further comprises determining whether the candidate agent specifically disrupts the CD93/MMRN2 interaction. In some embodiments, the method further comprises determining whether the candidate agent preferentially disrupts binding of CD93/IGFBP7 over CD93/MMRN2. In some embodiments, the method further comprises determining whether the candidate agent specifically disrupts binding the interaction between IGFBP7 and IGF-1, IGF-2, and/or IGF1R. In some embodiments, the method further comprises determining whether the candidate agent preferentially disrupts binding of CD93/IGFBP7 over IGFBP7/IGF-1, IGFBP-7/IGF-2, and/or IGFBP-7/IGF1R.

In some embodiments, there is provided a method of screening for an agent that is useful for treating cancer, comprising: a) providing a plurality of candidate agents; and b) identifying the candidate agent that specifically disrupts the CD93/IGFBP7 interaction, thereby obtaining an agent that is useful for treating cancer.

In some embodiments, there is provided a method of identifying an agent that specifically disrupts the CD93/IGFBP7 interaction, comprising: a) contacting a candidate agent with a CD93/IGFBP7 complex, and b) evaluating the effect of the candidate agent on the CD93/IGFBP7 complex, thereby identifying the agent that specifically disrupts the CD93/IGFBP7 interaction. In some embodiments, the method further comprises providing a CD93/IGFBP7 complex. In some embodiments, the method further comprises forming a CD93/IGFBP7 complex. In some embodiments, the CD93/IGFBP7 complex is present on a cell surface. In some embodiments, the CD93/IGFBP7 complex is present in an in vitro system.

In some embodiments, the CD93/IGFBP7 complex is non-naturally occurring. For example, the complex can comprise a variant of CD93 and/or a variant of IGFBP7. In some embodiments, the variant CD93 has a higher binding affinity to IGFBP7 than a wildtype CD93. In some embodiments, the variant IGFBP7 has a higher binding affinity to CD93 than a wildtype IGFBP7. Suitable CD93 variants and IGFBP7 variants include those described in the sections above. The present application in some embodiments also provides a non-naturally occurring CD93/IGFBP7 complex comprising any of the CD93 and/or IGFBP7 variants described herein. Such complex is useful for identifying candidate agents that disrupt the interaction of CD93 and IGFBP7.

In some embodiments, there is provided a method of identifying an agent that specifically disrupts the CD93/IGFBP7 interaction, comprising: a) contacting a candidate agent with CD93, and b) evaluating the interaction between the IGFBP7 and CD93, herein a reduced interaction as compared to a CD93 not contacted with the candidate agent is indicative that the agent specifically disrupts the CD93/IGFBP7 interaction. In some embodiments, the method further comprises providing a CD93. In some embodiments, the method further comprises providing an IGFBP7. Suitable CD93 include wildtype CD93 and variants thereof. Suitable IGFBP7 include wildtype IGFBP93 and variants thereof. Any of the CD93 and/or IGFBP7 variants described herein can be used for the identification method.

In some embodiments, there is provided a method of identifying an agent that specifically disrupts the CD37/IGFBP7 interaction, comprising: a) contacting a candidate agent with IGFBP7, and b) evaluating the interaction between the IGFBP7 and CD93, wherein a reduced interaction as compared to an IGFBP7 not contacted with the candidate agent is indicative that the agent specifically disrupts the CD93/IGFBP7 interaction. In some embodiments, the method further comprises providing an IGFBP7. In some embodiments, the method further comprises providing a CD93. In some embodiments, the method further comprises providing an IGFBP7. Suitable CD93 include wildtype CD93 and variants thereof. Suitable IGFBP7 include wildtype IGFBP93 and variants thereof. Any of the CD93 and/or IGFBP7 variants described herein can be used for the identification method.

Disruption in CD93/IGFBP7 binding activity, and/or CD93/IGFBP7 pathway activity may be measured by PCR. Taqman PCR, phage display systems, gel electrophoresis, reporter gene assay, yeast-two hybrid assay. Northern or Western analysis, immunohistochemistry, a conventional scintillation camera, a gamma camera, a rectilinear scanner, a PET scanner, a SPECT scanner, an MRI scanner, an NMR scanner, or an X-ray machine. The disruption may also be measured by using a method selected from label displacement, surface plasmon resonance, fluorescence resonance enemy transfer (FRET) or bioluminescence resonance energy transfer (BRET), fluorescence quenching, and fluorescence polarization.

The change in CD93/IGFBP7 binding activity and/or CD93/IGFBP7 pathway activity may be detected by detecting a change in the interaction between CD93 and IGFBP7, by detecting a change in the level of CD93 and/or IGFBP7, or by detecting a change in the level of one or more of the proteins in the CD93/IGFBP7 pathway. Cells in which the above described may be detected can be of a tumor origin, may be cultured cells, or may be obtained from or may be within a transgenic organism. Such transgenic organisms include, but are not limited to a mouse, rat, rabbit, sheep, cow or primate.

Screening assays of this application can include methods amenable to high-throughput screening of chemical libraries, making them particularly suitable for identifying small molecule drug candidates. The assays can be performed in a variety of formats, including protein-protein binding assays, biochemical screening assays, immunoassays, and cell-based assays, which are well characterized in the art. For in vitro screening, the agents can be identified by, e.g., phage display, GST-pull down, FRET (fluorescence resonance energy transfer), or BIAcore (surface plasmon resonance: Biacore AB, Uppsala, Sweden) analysis. For in vivo screening, agents can be identified by, e.g., yeast two-hybrid analysis, co-immunoprecipitation, co-localization by immunofluorescence, or FRET.

For screening experiments involving disruptions in the CD93/IGFBP7 interaction, cells expressing CD93 or IGFBP7 may be incubated in binding buffer with labeled IGFBP7 or CD93, respectively, in the presence or absence of increasing concentrations of a candidate agent. To validate and calibrate the assay, control competition reactions using increasing concentrations of unlabeled IGFBP7 or CD93, respectively, can be performed. After incubation, a washing step is performed to remove unbound IGFBP7 or CD93. Bound, labeled CD93 or IGFBP7 is measured as appropriate for the given label (e.g., scintillation counting, fluorescence, antibody-dye etc.). A decrease of at least 10% (e.g., at least 20%, 30%, 40%, 50%, or 60%) in the amount of labeled CD93 or IGFBP7 bound in the presence of candidate agent indicates displacement of binding by the candidate agent.

In some embodiments, candidate agent is considered to bind specifically in this or other assays described herein if they displace at least 10%, 20%, 30%, 40%, 50%, or preferably 60%, 70%, 80%, 90% or more of labeled CD93 or IGFBP7 at a concentration of 1 may or less. Of course, the roles of CD93 and IGFBP7 may be switched; the skilled person may adapt the method so CD93 is applied to IGFBP7 in the presence of various concentrations of candidate agent to determine disruptions in the CD93/IGFBP7 interaction.

Disruptions of the CD93/IGFBP7 interaction can be monitored by surface plasmon resonance (SPR). Surface plasmon resonance assays can be used as a quantitative method to measure binding between two molecules by the change in mass near an immobilized sensor caused by the binding or loss of binding of IGFBP7 from the aqueous phase to CD93 immobilized on the sensor (or vice versa). This change in mass is measured as resonance units versus time after injection or removal of the IGFBP7 or candidate agent and is measured using a Biacore Biosensor (Biacore AB). CD93 can be immobilized on a sensor chip (for example, research grade CM5 chip: Biacore AB) according to methods described by Salamon et al. (Salamon et al., 1996. Biophys J. 71: 283-294; Salamon et al., 2001. Biophys. J. 80: 1557-1567; Salamon et al., 1999. Trends Biochem. Sci. 24: 213-219, each of which is incorporated herein by reference for all purposes). Sarrio et al. demonstrated that SPR can be used to detect ligand binding to the GPCR A(1) adenosine receptor immobilized in a lipid layer on the chip (Sarrio et al., 2000, Mol. Cell. Biol. 20, 5164-5174, incorporated herein by reference for all purposes). Conditions for IGFBP7 binding to CD93 in an SPR assay can be fine-tuned by one of skill in the art using the conditions reported by Sarrio et al. as a starting point.

SPR can assay for inhibitors of binding in at least two ways. First. IGFBP7 can be pre-bound to immobilized CD93, followed by injection of candidate agent at a concentration ranging from 0.1 nM to 1 pM. Displacement of the bound IGFBP7 can be quantitated, permitting detection of inhibitor binding. Alternatively, the chip bound CD93 can be pre-incubated with candidate agent and challenged with IGFBP7. A difference in IGFBP7 binding to CD93 exposed to inhibitor relative to that on a chip not pre-exposed to inhibitor will demonstrate binding or displacement of IGFBP7 in the presence of CD93. In either assay, a decrease of 10% (e.g., 20%, 30%, 40%, 50%, 60%, 70%, 80%, 90%) or more in the amount of IGFBP7 bound in the presence of candidate agent, relative to the amount of an IGFBP7 bound in the absence of candidate agent that the candidate agent inhibits the interaction of CD93 and IGFBP7. While CD93 is immobilized in the above, the skilled person may readily adapt the method so that IGFBP7 is the immobilized component.

Another method of detecting agents that inhibit binding of CD93/IGFBP7 interaction uses fluorescence resonance energy transfer (FRET). FRET is a quantum mechanical phenomenon that occurs between a fluorescence donor (D) and a fluorescence acceptor (A) in close proximity to each other (usually 100 angstroms of separation) if the emission spectrum of D overlaps with the excitation spectrum of A. The molecules to be tested, e.g., CD93 and IGFBP7, are labeled with a complementary pair of donor and acceptor fluorophores. While bound closely together by the CD93/IGFBP7 interaction, the fluorescence emitted upon excitation of the donor fluorophore will have a different wavelength than that emitted in response to that excitation wavelength when the CD93 and IGFBP7 are not bound, providing for quantitation of bound versus unbound molecules by measurement of emission intensity at each wavelength. Donor fluorophores with which to label the CD93 or IGFBP7 are well known in the art. Examples include variants of the A. victoria GFP known as Cyan FP (CFP, Donor (D)) and Yellow FP (YFP, Acceptor(A)).

In some embodiments, the addition of a candidate agent to the mixture of labeled IGFBP7 and YFP-CD93 will result in an inhibition of energy transfer evidenced by, for example, a decrease in YIP fluorescence relative to a sample without the candidate agent. In an assay using FRET for the detection of CD93/IGFBP7 interaction, a 10% or greater (e.g. equal to or more than 20%, 30%, 40%, 50%, 60%, 70%, 80%, or 90%) decrease in the intensity of fluorescent emission at the acceptor wavelength in samples containing a candidate agent, relative to samples without the candidate agent, indicates that the candidate agent inhibits the CD93/IGFBP7 interaction. Conversely, a 10% or greater (e.g., equal to or more than 20%, 30%, 40%, 50%, 60%, 70%, 80%, 90%) increase in the intensity of fluorescent emission at the acceptor wavelength in samples containing a candidate agent, relative to samples without the candidate agent indicates that the candidate agent induces a conformational change and enhance the CD93/IGFBP7 interaction.

A variation on FRET uses fluorescence quenching to monitor molecular interactions. One molecule in the interacting pair can be labeled with a fluorophore, and the other with a molecule that quenches the fluorescence of the fluorophore when brought into close apposition with it. A change in fluorescence upon excitation is indicative of a change in the association of the molecules tagged with the fluorophore quencher pair. Generally, an increase in fluorescence of the labeled CD93 is indicative that the IGFBP7 molecule hearing the quencher has been displaced. Of course, a similar effect would arise when IGFBP7 is fluorescently labeled and CD93 bears the quencher. For quenching assays, a 10% or greater increase (e.g., equal to or more than 20%, 30%, 40%, 50%, 60%, 70%, 80%, or 90%) in the intensity of fluorescent emission in samples containing a candidate agent, relative to samples without the candidate agent, indicates that the candidate agent inhibits CD93/IGFBP7 interaction. Conversely, a 10% or greater decrease (e.g., equal to or more than 20%, 30%, 40%, 50%, 60%, 70%, 80%, or 90%) in the intensity of fluorescent emission in samples containing a candidate agent, relative to samples without the candidate agent, indicates that the candidate induces a conformational change and enhance the CD93/IGFBP7 interaction.

In addition to the surface plasmon resonance and FRET methods fluorescence polarisation measurement is useful to quantitate binding. The fluorescence polarisation value for a fluorescently-tagged molecule depends on the rotational correlation time or tumbling rate. Complexes, such as those formed by CD93 or IGFBP7 associating with a fluorescently labeled IGFBP7 or CD93, respectively, have higher polarization values than uncomplexed, labeled IGFBP7 or CD93, respectively. The inclusion of a candidate agent of the CD93/IGFBP7 interaction results in a decrease in fluorescence polarization, relative to a mixture without the candidate agent, if the candidate agent disrupts or inhibits the interaction of CD93/IGFBP7. Fluorescence polarization is well suited for the identification of small molecules that disrupt the formation of complexes. A decrease of 10% or more (e.g., equal to or more than 20%, 30%, 40%, 50%, 60%) in fluorescence polarization in samples containing a candidate agent, relative to fluorescence polarization in a sample lacking the candidate agent, indicates that the candidate agent inhibits CD93/IGFBP7 interaction.

Another detection system is bioluminescence resonance energy transfer (BRET), which uses light transfer between fusion proteins containing a bioluminescent luciferase and a fluorescent acceptor. In general, one molecule of the CD93/IGFBP7 interacting pair is fused to a luciferase (e.g. Renilla luciferase (Rluc))—a donor which emits light in the wavelength of −395 nm in the presence of luciferase substrate (e.g. DeepBlueC). The other molecule of the pair is fused to an acceptor fluorescent protein that can absorb light from the donor, and emit light at a different wavelength. An example of a fluorescent protein is GFP (green fluorescent protein) which emits light at ˜5 10 nm. The addition of a candidate agent to the mixture of donor fused-IGFBP7 and acceptor-fused-CD93 (or vice versa) will result in an inhibition of energy transfer evidenced by, for example, a decrease in acceptor fluorescence relative to a sample without the candidate agent. In an assay using BRET for the detection of CD93/IGFBP7 interaction, a 10% or greater (e.g. equal to or more than 20%, 30% 40%, 50%, 60%, 70%, 80%, or 90%) decrease in the intensity of fluorescent emission at the acceptor wavelength in samples containing a candidate agent, relative to samples without the candidate agent, indicates that the candidate agent inhibits the CD93/IGFBP7 interaction. Conversely, a 10% or greater (e.g. equal to or more than 20%, 30%, 40%, 50%, 60%, 70%, 80%, or 90%) increase in the intensity of fluorescent emission at the acceptor wavelength in samples containing a candidate agent, relative to samples without the candidate agent, indicates that the candidate agent induces a conformational change and enhance the CD93/IGFBP7 interaction.

It should be understood that any of the binding assays described herein can be performed with any ligand other than CD93 and IGFBP7 (for example, agonist, antagonist, etc.) that binds to CD93 or IGFBP7, e.g., a small molecule identified as described herein or CD93 or IGFBP7 mimetics including but not limited to any of natural or synthetic peptide, a polypeptide, an antibody or antigen-binding fragment thereof, a lipid, a carbohydrate, and a small organic molecule.

Any of the binding assays described can be used to determine the presence of an inhibitor in a sample, e.g., a tissue sample, that binds to CD93 or IGFBP7, or that affects the binding of CD93 and IGFBP7. To do so, CD93 is reacted with IGFBP7 in the presence or absence of the sample, and binding is measured as appropriate for the binding assay being used. A decrease of 10% or more (e.g., equal to or more than 20%, 30%, 40%, 50%, 60%, 70%, 80% or 90%) in the binding of CD93/IGFBP7 indicates that the sample contains an inhibitor that blocks CD93/IGFBP7 interaction.

Any of the binding assays described can also be used to determine the presence of an inhibitor in a library of compounds. Such screening techniques using, for example, high throughput screening are well known in the art.

The present application also provides methods for identifying an agent capable of inhibiting the CD93/IGFBP7 signaling pathway, wherein the method comprises measuring the signaling response induced by the CD93/IGFBP7 interaction in the presence of said agent, and comparing it with the signaling response induced by the CD93/IGFBP7 interaction in the absence of said agent. In some embodiments, said method comprises the steps of: a) contacting CD93 with IGFBP7 in the presence and absence of a test agent under conditions permitting the interaction of CD93 and IGFBP7; and b) measuring a signaling response induced by the CD93/IGFBP7 interaction, wherein a change in response in the presence of the test agent of at least about 10% (such as at least about 10%, 20%, 30%, 40%, 50%, 60%, 70%, 80%, or 90%) compared with the response in the absence of the test agent indicates the test agent is identified as capable of inhibiting the CD93/IGFBP7 interaction.

The present application provides a method for identifying a CD93 or IGFBP7 mimetic, which mimetic has the same, similar or improved functional effect as CD93 or IGFBP7 in the interaction with IGFBP7 or CD93, wherein the method comprises measuring the interaction with IGFBP7 or CD93 by a candidate mimetic. In some embodiments, said method comprises: a) contacting CD93 or IGFBP7 with a candidate mimetic under conditions permitting the interaction of the mimetic with CD93 or IGFBP7; and b) measuring interaction of the mimetic with CD93 or IGFBP7, wherein the interaction is at least about 10% (such as about 20%, 30%, 40%, 50%, 60%, 70%, 80%, or 90%) of that observed for the CD93/IGFBP7 interactions, distinguishes the candidate mimetic as a CD93 or IGFBP7 mimetic of the application.

Furthermore, the present application also provides a method for identifying a CD93 or IGFBP7 mimetic, which mimetic has the same, similar or improved functional effect as CD93 or IGFBP7 in interaction with IGFBP7 or CD93 respectively, wherein the method comprises measuring the signaling response induced by the CD93 or IGFBP7-mimetic interaction and comparing it with the signaling response induced by CD93/IGFBP7 interaction. In some embodiments, said method comprises: a) contacting CD93 or IGFBP7 with a candidate mimetic under conditions permitting the interaction of the mimetic with CD93 or IGFBP7; and b) measuring a signaling response induced by the CD93 or IGFBP7-mimetic interaction, wherein a signaling response that is at least about 10% (such as about 20%, 30%, 40%, 50%, 60%, 70%, 80%, or 90%) of that observed for the CD93/IGFBP7 interactions, distinguishes the candidate mimetic as a CD93 or IGFBP7 mimetic of the application.

The measuring of mimetic signaling activity of interaction with CD93 or IGFBP7 can be performed by methods described herein for other assays, such as SPR and FRET. Any of the binding assays described can be used to determine the presence of a mimetic in a sample, e.g., a tissue sample that binds to CD93 or IGFBP7. To do so, CD93 or IGFBP7 is reacted in the presence or absence of the sample, and signaling is measured as appropriate for the assay being used. An increase of about 10% or more (e.g., equal to or more than about 20%, 30%, 40% 50%, 60%, 70%, 80%, or 90%) in the signaling of CD93 or IGFBP7 indicates that the sample contains a mimetic that binds to CD93 or IGFBP7.

Any of the signaling assays described can also be used to determine the presence of a mimetic in a library of compounds. Such screening techniques using, for example, high throughput screening are well known in the art.

The candidate or test compounds or agents of or employed by the present application can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al. (1997) Anticancer Drug Des. 12; 145, incorporated by reference in its entirety for all purposes).

Examples of methods for the synthesis of molecular libraries can be found in the art, for example in: DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233, each of which are incorporated by reference in their entirety for all purposes. Libraries of compounds may be presented in solution (e.g. Houghten (1992) Biotechniques 13: 412), or on beads (Lam (1991) Nature 354: 82), chips (Fodor (1993) Nature 364: 555), bacteria (Ladner, U.S. Pat. No. 5,223,409), spores (Ladner '409), plasmids (Cull et al. (1992) Proc Natl Acad Sci USA 89: 1865) or on phage (Scott and Smith (1990) Science 249: 386); (Devlin (1990) Science 249: 404); (Cwirla et al. (1990) Proc. Natl. Acad. Sci. 87: 6378); (Felici (1991) J. Mol. Biol. 222: 301); (Ladner, supra), each of which are incorporated by reference in their entirety for all purposes.

In some embodiments, there is provided a cell-based assay comprising contacting a cell expressing a CD93 or IGFBP7 with a candidate or test compound or agent, and determining the ability of the test compound to inhibit the activity of said CD93 or IGFBP7. Determining the ability of the test compound to inhibit the CD93/IGFBP7 interaction can be accomplished, for example, by determining the ability of the candidate or test compound or agent to inhibit CD93/IGFBP7 interaction.

Determining the ability of candidate or test compounds or agents to inhibit a CD93/IGFBP7 signaling pathway can be accomplished by determining direct binding. These determinations can be accomplished, for example, by coupling the CD93 or IGFBP7 with a radioisotope or enzymatic label such that binding of the protein to a candidate or test compound or agent can be determined by detecting the labeled protein in a complex. For example, molecules, e.g., proteins, can be labeled with ¹²⁵I, ³⁵S, ¹⁴C, or ³H, either directly or indirectly, and the radioisotope detected by direct counting of radio emmission or by scintillation counting. Alternatively, molecules can be enigmatically labeled with, for example, horseradish peroxidase, alkaline phosphatase, or luciferase, and the enzymatic label detected by determination of conversion of an appropriate substrate to product.

It is also within the scope of the application to determine the ability of candidate or test compounds or agents to inhibit the CD93/IGFBP7 interaction, without the labeling of any of the interactants. For example, a microphysiometer can be used to detect the interaction of test compounds with CD93 or IGFBP7 without the labeling of any of the interactants (McConnell et al. (1992) Science 257: 1906 incorporated by reference in its entirety for all purposes). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In some embodiments, there is provided a cell-free assay in which a protein or biologically active portion thereof is contacted with a candidate or test compound or agent (e.g., or a compound tested for its ability to inhibit the CD93/IGFBP7 interaction) and the ability of the test compound to bind to CD93 or IGFBP7, or biologically active portions thereof, is determined. Binding of the test compound to CD93 or IGFBP7 can be determined either directly or indirectly as described above.

Such a determination may be accomplished using a technology such as real-time Biomolecular Interaction Analysis (BIA). Sjolander et al. 1991 Anal. Chem. 63:2338-2345 and Szabo et al., 1995 Curr. Opin. Struct. Biol. 5:699-705, each of which are incorporated by reference in their entirety for all purposes. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In some embodiments of the above assay methods of the present application, it may be desirable to immobilize CD93 or IGFBP7 to facilitate separation of complexed from uncomplexed forms of the protein, as well as to accommodate automation of the assay. Binding of a test compound to CD93 or IGFBP7 can be accomplished in any vessel suitable for containing the reactants. Examples of such vessels include microtitre plates, test tubes, and microcentrifuge tubes. In some embodiments, a fusion protein can be provided which adds a domain that allows the protein to be bound to a matrix. For example, glutathione-S-transferase/kinase fusion proteins or glutathione-S-transferase/target fusion proteins can be adsorbed onto glutathione sepharose beads (Sigma Chemical. St. Louis. Mo.) or glutathione derivatized microtitre plates, which are then combined with the test compound or the test compound and the non-adsorbed CD93 or IGFBP7, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding determined using standard techniques.

Other techniques for immobilizing proteins on matrices can also be used in the screening assays of the application. For example, CD93 or IGFBP7 can be immobilized utilizing conjugation of biotin and streptavidin, Biotinylated CD93 or IGFBP7 or target molecules can be prepared from biotin-NHS (N hydroxy-succinimide) using techniques well known in the art (e.g., biotinylation kit, Pierce Chemicals, Rockford, Ill.), and immobilized in the wells of streptavidin-coaled 96 well plates (Pierce Chemical). Alternatively, antibodies reactive with CD93 or IGFBP7 or target molecules can be derivatized to the wells of the plate, and unbound CD93 or IGFBP7 trapped in the wells by antibody conjugation. Methods for detecting such complexes, in addition to those described above for the GST-immobilized complexes, include immunodetection of complexes using antibodies reactive with CD93 or IGFBP7 or target molecules.

In some embodiments, the CD93 or IGFBP7 can be used as “bait proteins” in a two-hybrid assay or three-hybrid assay (see, e.g., U.S. Pat. No. 5,283,317; Zervos et al., 1993 Cell 72:223-232; Madura et al., 1993 J. Biol. Chem. 268:12046-12054; Bartel et al., 1993 Biotechniques 14:920-924; Iwahuchi et al., 1993 Oncogene 8:1693-1696; and Brent WO94/10300), each of which are incorporated by reference in their entirety for all purposes, to identify other proteins which bind to CD93 or IGFBP7.

The two-hybrid system is based on the modular nature of most transcription factors, which consist of separable DNA-binding and activation domains. Briefly, the assay utilizes two different DNA constructs. In one construct, the gene that codes for CD93 or IGFBP7 is fused to a gene encoding the DNA binding domain of a known transcription factor (e.g., GAL-4). In the other construct, a DNA sequence from a library of DNA sequences, that encode an unidentified protein (“prey” or “sample”) is fused to a gene that codes for the activation domain of the known transcription factor. If the “bait” and the “prey” proteins are able to interact, in viva, forming a kinase dependent complex, the DNA-binding and activation domains of the transcription factor are brought into close proximity. This proximity allows transcription of a reporter gene LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with CD93 or IGFBP7.

It is to be understood that the protein-protein interaction assays described herein can also be useful for determining if an agent blocks interaction between CD93 or IGFBP7 and other binding partners, for example the interaction between CD93 and MMNR2 and the interaction between IGFBP7 and IGF-1, IGF-2, or IGF1R.

Also provided are agents identified by any of the methods described herein. Accordingly, it is within the scope of the application to further use an agent identified as described herein in an appropriate animal model. For example, an agent identified as described herein (e.g., an agent capable of blocking the CD93/IGFBP7 interaction) can be used in an animal model to determine the efficacy, toxicity, or side effects of treatment with such an agent. Alternatively, an agent identified as described herein can be used in an animal model to determine the mechanism of action of such an agent. Furthermore, this application pertains to uses of novel agents identified by the above-described screening assays for treatments as described herein.

### V. Methods of Preparation, Nucleic Acids, Vectors, Host Cells, and Culture Medium

In some embodiments, there is provided a method of preparing the CD93/IGFBP7 blocking agents (such as anti-CD93 antibodies, anti-IGFBP7 antibodies, inhibitory CD93 polypeptides, inhibitory IGFBP7 polypeptides as described herein) and composition comprising the agents, nucleic acid construct, vector, host cell, or culture medium that is produced during the preparation of the agents.

### Polypeptide Expression and Production

The polypeptides (e.g., anti-CD93 or anti-IGFBP7 antibodies, e.g., inhibitory CD93 or IGFBP7 polypeptides) described herein can be prepared using any known methods in the art, including those described below and in the Examples.

### Monoclonal Antibodies

Monoclonal antibodies are obtained from a population of substantially homogeneous antibodies, i.e., the subject antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature. 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103, incorporated by reference in its entirety for all purposes.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalised myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif., USA, and SP-2 cells (and derivatives thereof, e.g., X63-Ag8-653) available from the American Type Culture Collection. Manassas, Va. USA. Human myeloma and mouse-human heteromyeloma cell lines also have been described for the production of human monoclonal antibodies (Kozbor. J. Immunol., 133:3001 (1984); Brodeur et al., Monoclonal Antibody Production Techniques and Applications, pp. 51-63 (Marcel Dekker, Inc. New York, 1987), each of which are incorporated by reference in their entirety for all purposes).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

Alter hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitable separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies) The hybridoma cells serve as a preferred source of such DNA. Once isolated, the DNA may be placed into expression sectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al. Curr. Opinion in Immunol., 5, 256-262 (1993) and Pluckthun, Immunol. Revs. 130:151-188 (1992).

In a further embodiment, antibodies can be isolated from antibody phage libraries generated using the techniques described in McCafferty el al., Nature. 348:552-554 (1990). Clackson et al., Nature. 352:624-628 (1991) and Marks et al., J. Mol. Biol., 222:581-597 (1991), each of which are incorporated by reference in their entirety for all purposes, describe the isolation of murine and human antibodies, respectively, using phage libraries. Subsequent publications describe the production of high affinity (nM range) human antibodies by chain shuffling (Marks et al., Bio/Technology, 10:779-783 (1902)), as well as combinatorial infection and in vivo recombination as a strategy for constructing yen large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA. 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Acid Molecules Encoding Polypeptides

In some embodiments, there is provided a polynucleotide encoding any one of the antibodies (such as anti-CD93 or anti-IGFBP7 antibodies) or polypeptides (such as inhibitory CD93 or IGFBP7 polypeptides) described herein. In some embodiments, there is provided a polynucleotide prepared using any one of the methods as described herein. In some embodiments, a nucleic acid molecule comprises a polynucleotide that encodes a heavy chain or a light chain of an antibody (e.g., anti-CD93 or anti-IGFBP7 antibody). In some embodiments, a nucleic acid molecule comprises a polynucleotide that encodes an inhibitory CD93 polypeptide or an inhibitory IGFBP7 polypeptide. In some embodiments, a nucleic acid molecule comprises both a polynucleotide that encodes a heavy chain and a polynucleotide that encodes a light chain, of an antibody (e.g., anti-CD93 or anti-IGFBP7 antibody). In some embodiments, a first nucleic acid molecule comprises a first polynucleotide that encodes a heavy chain and a second nucleic acid molecule comprises a second polynucleotide that encodes a light chain. In some embodiments, a nucleic acid molecule encoding a scFv (e.g., anti-CD93 or anti-IGFBP7 scFv) is provided. In some embodiments, a nucleic acid molecule comprises a polynucleotide that encodes an inhibitory CD93 polypeptide or an inhibitory IGFBP7 polypeptide.

In some such embodiments, the heavy chain and the light chain of an antibody (e.g., anti-CD93 or anti-IGFBP7 antibody) are expressed from one nucleic acid molecule, or from two separate nucleic acid molecules, as two separate polypeptides. In some embodiments, such as when an antibody is a scFv, a single polynucleotide encodes a single polypeptide comprising both a heavy chain and a light chain linked together.

In some embodiments, a polynucleotide encoding a heavy chain or light chain of an antibody (e.g., anti-CD93 or anti-IGFBP7 antibody) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

In some embodiments, there is provided a vector comprising any polynucleotides that encode the heavy chains and/or light chains of any one of the antibodies (e.g., anti-CD93 or anti-IGFBP7 antibodies) described herein. In some embodiments, there is provided a vector comprising any polynucleotides that encode polypeptides (e.g., inhibitory CD93 or IGFBP7 polypeptides) described herein. In some embodiments, there is provided a vector comprising any nucleic acid construct described herein. In some embodiments, there is provided a vector prepared using am method described herein. Vectors comprising polynucleotides that encode any of polypeptides (such as anti-CD93 or anti-IGFBP7 antibodies or inhibitory CD93 or IGFBP7 polypeptides) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides.

In some embodiments, a first vector comprises a polynucleotide that encodes a heavy chain of an antibody (e.g., anti-CD93 or anti-IGFBP7 antibody) and a second vector comprises a polynucleotide that encodes a light chain of an antibody (e.g., anti-CD93 or anti-IGFBP7 antibody). In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a Vector is selected that is optimised for expression of polypeptides in CHO or CHO-derived cells, or in NSO cells. Exemplary such vectors are described, e.g., in Running Deer et al. Biotechnol. Prog. 20:880-889 (2004).

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. The viral vector may be a plasmid or a transposon (such as a PiggyBac- or a Sleeping Beauty transposon),

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of polypeptides (such as antibodies or inhibitory polypeptides) described herein under a fermentation condition.

In some embodiments, the polypeptides described herein (e.g., anti-CD93 or anti-IGFBP7 antibodies or inhibitory CD93 or IGFBP7 polypeptides) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6F, cells; CHO cells, including CHO-S, DG44, Lec13 CHO cells, and FUT8 CHO cells; PER.C6® cells (Crucell); and NSO cells. In some embodiments, the polypeptides described herein (e.g., anti-CD93 or anti-IGFBP7 antibodies or inhibitory CD93 or IGFBP7 polypeptides) may be expressed in yeast See, e.g., U S. Publication No. US 2006/0270045 A1. In some embodiments, a particular eukaryotic host cell is selected based on its ability to make desired post-translational modifications to the heavy chains and/or light chains of the desired antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning. A Laboratory Manual. 3rd ed. Cold Spring Harbor Laboratory Press (2001), incorporated by reference in its entirety for all purposes. Nucleic acids may be transiently or stably transfected in the desired host cells, according to any suitable method.

The invention also provides host cells comprising any of the polynucleotides or vectors described herein. In some embodiments, the invention provides a host cell comprising an anti-CD93 or anti-IGFBP7 antibody. Any host cells capable of over-expressing heterologous DNAs can be used for the purpose of isolating the genes encoding the antibody, polypeptide or protein of interest. Non-limiting examples of mammalian host cells include but not limited to COS. HeLa and CHO cells. See also PCT Publication No. WO 87/04462. Suitable non-mammalian host cells include prokaryotes (such as E. coli or B. subtilis) and yeast (such as S. cerevisae, S. pombe; or K. lactis).

In some embodiments, the polypeptide is produced in a cell-free system. Non-limiting exemplary cell-free systems are described, e.g., in Sitaraman et al., Methods Mol. Biol. 498: 220-44 (2009); Spirin, Trends Biotechnol. 22: 538-45 (2004); Endo et al., Biotechnol. Adv. 21: 603-713 (2003).

### Culture Medium

In some embodiments, there is provided a culture medium comprising any polypeptide, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g. HAT medium). In some embodiments, the medium does not comprise serum. In some embodiments, the medium comprises serum. In some embodiments, the medium is a D-MEM or RPMI-1640 medium.

### Purification of Polypeptides

The polypeptides (e.g., anti-CD93 or anti-IGFBP7 antibodies, e.g., inhibitory CD93 or IGFBP7 polypeptides) may be purified by am suitable method Such methods include, but are not limited to, the use of affinity matrices or hydrophobic interaction chromatography. Suitable affinity ligands include the ROR1 ECD and ligands that bind antibody constant regions. In some embodiments, a Protein A, Protein G, Protein A/G, or an antibody affinity column may be used to bind the constant region and to purify an antibody comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some pot peptides such as antibodies. Many methods of purifying polypeptides are known in the art.

### VI. Compositions, Kits, and Articles of Manufacture

The present application also provides compositions, kits, medicines, and unit dosage forms for use in any of the methods described herein.

### Compositions

Any of the CD93/IGFBP7 blocking agents described herein can be present in a composition (such as a formulation) that includes other agents, excipients, or stabilizers.

In some embodiments, the composition further comprises a target agent or a carrier that promotes the delivery of the CD93/IGFBP7 blacking agent to a tumor tissue or a tissue associated with abnormal vascular or hypoxia. Exemplary carriers include liposomes, micelles, nanodisperse albumin and its modifications, polymer nanoparticles, dendrimers, inorganic nanoparticles of different compositions.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the CD93/IGFBP7 blocking agent. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcry stalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing. In addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxy benzoates, talc, magnesium stearate, and mineral oil. In some embodiments, the composition comprising the CD93/IGFBP7 blocking agents with a carrier as discussed herein is present in a dry formulation (such as lyophilized composition). The formulations can additionally include lubricating agents, welting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilisers, and preservatives. The formulations can be presented in unit-dose or multi-dose sealed containers, such as ampules and vials, and can be stored in a freeze-dried (lyophilised) condition requiring only the addition of the sterile liquid excipient, for example, water, for injections, immediately prior to use. Extemporaneous injection solutions and suspensions can be prepared from sterile powders, granules, and tablets of the kind preciously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits

Kits provided herein include one or more containers comprising the CD93/IGFBP7 blocking agent or a pharmaceutical composition comprising the CD93/IGBP7 blocking agent described herein and/or other agent(s), and in some embodiments, further comprise instructions for use in accordance with any of the methods described herein. The kit may further comprise a description of selection of subject suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising a CD93/IGFBP7 blocking agent comprising an anti-CD93 antibody, or a pharmaceutically acceptable salt thereof and a pharmaceutically acceptable carrier; and optionally b) instructions for administering the CD93/IGFBP7 blocking agent for treatment of a disease or condition. In some embodiments, the kit comprises a) a composition comprising a CD93/IGFBP7 blocking agent comprising an anti-IGFBP7 antibody, or a pharmaceutically acceptable salt thereof and a pharmaceutically acceptable carrier; and optionally b) instructions for administering the CD93/IGFBP7 blocking agent for treatment of a disease or condition. In some embodiments, the kit comprises a) a composition comprising a CD93/IGFBP7 blocking agent comprising an inhibitory CD93 polypeptide, or a pharmaceutically acceptable salt thereof and a pharmaceutically acceptable carrier; and optionally b) instructions for administering the CD93/IGFBP7 blocking agent for treatment of a disease or condition. In some embodiments, the kit comprises a) a composition comprising a CD93/IGFBP7 blocking agent comprising an inhibitory IGFBP7 polypeptide, or a pharmaceutically acceptable salt thereof and a pharmaceutically acceptable carrier; and optionally b) instructions for administering the CD93/IGFBP7 blocking agent for treatment of a disease or condition.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In some embodiments, the kits comprise one or more components that facilitate delivery of the CD93/IGFBP7 blocking agent, or a composition comprising the agent, and/or additional therapeutic agents to the subject. In some embodiments, the kit comprises, e.g., syringes and needles suitable for delivery of cells to the subject, and the like. In such embodiments, the CD93/IGFBP7 blocking agent, or a composition comprising the agent may be contained in the kit in a bag, or in one or more vials. In some embodiments, the kit comprises components that facilitate intravenous or intra-arterial delivery of the CD93/IGFBP7 blocking agent, or a composition comprising the agent to the subject. In some embodiments, the CD93/IGFBP7 blocking agent, or a composition comprising the agent may be contained, e.g., within a bottle or bag (for example, a blood bag or similar bag able to contain up to about 1.5 L solution comprising the cells), and the kit further comprises tubing and needles suitable for the delivery of the CD93/IGFBP7 blocking agent, or a composition comprising the agent to the subject.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the zinc as disclosed herein to provide effective treatment of a subject for an extended period, such as any of 1 day, 2 days, 3 days, 4 days, 5 days, 6 days, 7 days, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

### EXAMPLES

The examples below are intended to be purely exemplary of the application and should therefore not be considered to limit the invention in any way. The following examples and detailed description are offered by way of illustration and not by way of limitation.

### Example 1

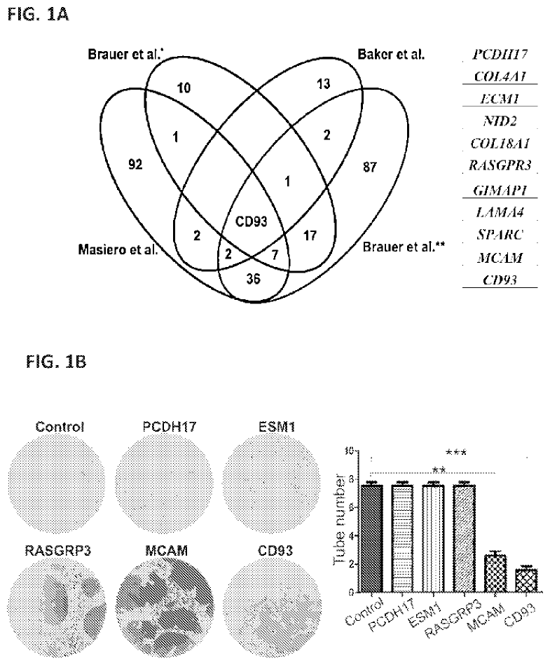

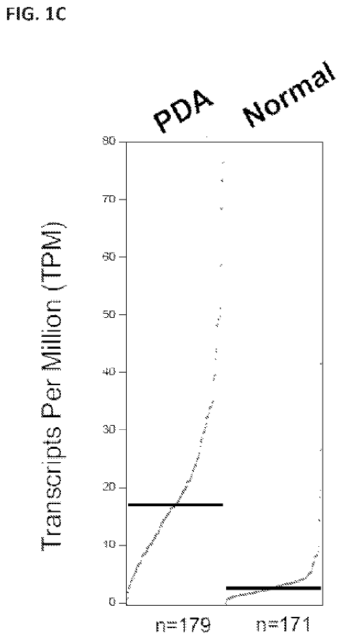

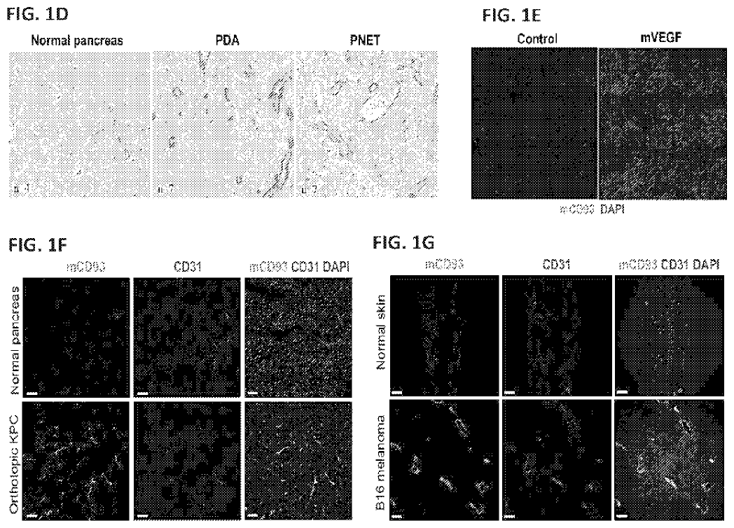

To identify new targets which could be responsible for VEGF inhibitor-induced vascular normalisation, gene expression profiles were studied in tumor ECs under the treatment of VEGF inhibitors in viva from four recently published RNA-Seq datasets (28-31). Three databases were from xenograft tumor models treated with VEGF inhibitors, and one was from human neuroendocrine tumors. Genes which were consistently reduced across multiple datasets with a cutoff log₂ fold change <−0.5 were sorted out. Eleven genes whose expressions were significantly reduced by VEGF inhibitors in at least three datasets were identified (FIG. 1A). Most of them are transmembrane proteins or extracellular matrix proteins (Sec Table 2). Five candidate genes upregulated in tumor ECs were selected their functions were tested in a tube formation assay using freshly isolated human endothelial cells from blood vessels (HUVEC). Among them, knockdown of CD93 genes led to significant reductions of tube formation in HUVEC cells (FIG. 1B).

TABLE 2

| | Additional | Location | EC | Tumor EC | |
|---|---|---|---|---|---|
| Gene | name | (Uniprot) | specificity | expression | Reference |
| PCDH17 | Protocadherin 17 | Plasma | Yes | Upregulated | Ghilardi C., |
| | | membrane | | | et al, 2010 |
| COL4A1 | | ECM | No | No | |
| ESM1 | | ECM | Yes | Upregulated | Leroy X., |
| | | | | | et al, 2010 |
| | | | | | Abid M R., |
| | | | | | et al, 2006 |
| NID2 | Osteonidogen. | ECM | No | unclear | |
| | Nidogen-2 | | | | |
| COL18A1 | Endostatin | ECM | No | No | |
| RASGRP3 | GRP3 | Plasma | Yes | Upregulated | Roberts |
| | | membrane | | | D M., |
| | | | | | et al, 2004 |
| GIMAP1 | GIMAP | Golgi | Yes | unclear | |
| LAMA4 | | ECM | No | Upregulated | |
| SPARC | Osteonectin | ECM | No | unclear | |
| MCAM | CD146. | Plasma | Yes | Upregulated | Wragg J W, |
| | MUC18 | membrane | | | 2016 |
| CD93 | C1qR. AA4.1 | Plasma | Yes | Upregulated | Lugano R., |
| | | membrane | | | et al. 2018 |

Analysis of the Cancer Genome Atlas (“the TCGA”) database for pancreatic cancer revealed that CD93 transcription is significantly higher in pancreatic ductal adenocarcinoma (PDA) than in normal pancreas (FIG. 1C). Furthermore, CD93 protein was clearly upregulated on blood vessels within PDA and pancreatic neuroendocrine tumors (PNET), two main tumor types in pancreas (FIG. 1D).

CD93 expression was also evaluated in mouse normal tissues and tumors. Freshly isolated aortic endothelial cells (MAECs) express negligible CD93 but it could be upregulated by incubation with VEGF, confirming that VEGF signaling directly regulates CD93 expression (FIG. 1E). In mouse normal pancreas and skin, blood s vessels express very low levels of CD93, as revealed by co-immunofluorescence staining of CD93 and CD31. Interestingly, the expression of CD93 in tumor vasculatures was drastically increased in an orthotopic KPC model and in a B16 melanoma model (FIGS. 1F and 1G). These results show that CD93 is upregulated in tumor vasculature selectively and this could be due to the exposure to VEGF in the tumor microenvironment (“the TME”).

### Example 2

To evaluate the possible effect of CD93 in vivo, a mAb (clone 7C10, rat IgG) specific for mouse CD93 was generated by immunizing a rat with mouse CD93 fusion protein. C57BL/6 mice were implanted with KPC tumor line derived from KPC transgenic mice (36). When tumors became palpable, mice were treated with 7C10 twice a week for two weeks. The 7C10 alone was able to slow KPC tumor growth by about 60% (FIG. 2A). The IF staining of tumor tissues did not show a clear change of CD31− microvessel density upon 7C10 treatment. However, the vascular length was increased significantly more than 1.8-fold, and there was a 3-fold increase in the percentages of blood vessels with circular shape in tumors treated with 7C10 (FIG. 2B). Moreover, after the treatment, there was approximately a 3.5-fold increase than the control of pericyte-covered blood vessels, based on co-staining of NG2 and CD31 (FIG. 2C). In line with this observation, there were over twice as many as alpha smooth muscle actin (α-SMA)-positive cells associated with blood vessels within 7C10-treated tumors (FIG. 2D).

To determine whether the structural changes in tumor vasculature can translate into functional improvement, tumor vessel perfusion in response to CD93 blockade was examined. Tumor-bearing mice mentioned above undergoing one week of antibody treatment w ere intravenously (i.v.) injected with lectin-FITC before sacrificing. It was found that in control tumors, few blood vessels located at the edge of the tumors were FITC-positive, while in tumors treated with 7C10, the majority of vessels in both the center and edge of tumors were stained with FITC-lectin (FIG. 2E). There were significantly more FITC-positive micros vessels in 7C10-treated tumors than the control (75% vs 20%). In summary, the results support that targeting CD93 could normalize tumor vasculature and promotes vascular maturation and perfusion in tumors.

### Example 3

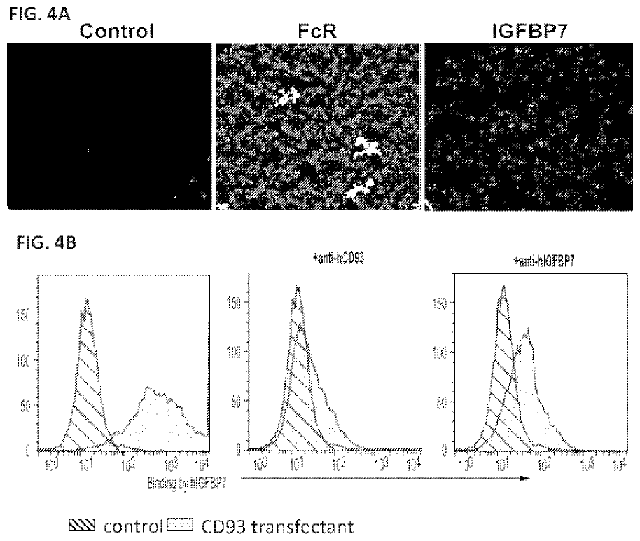

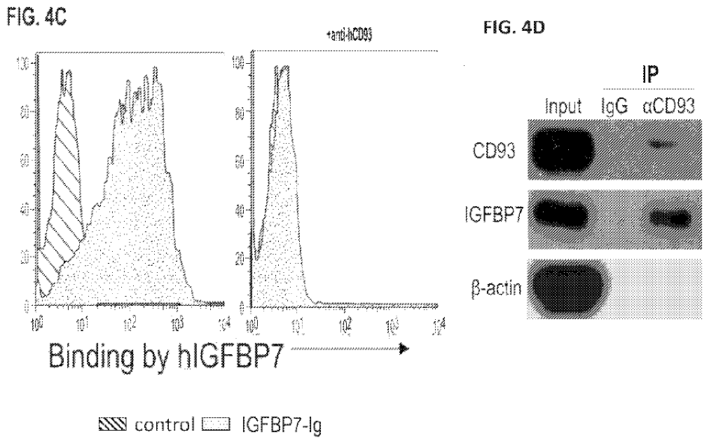

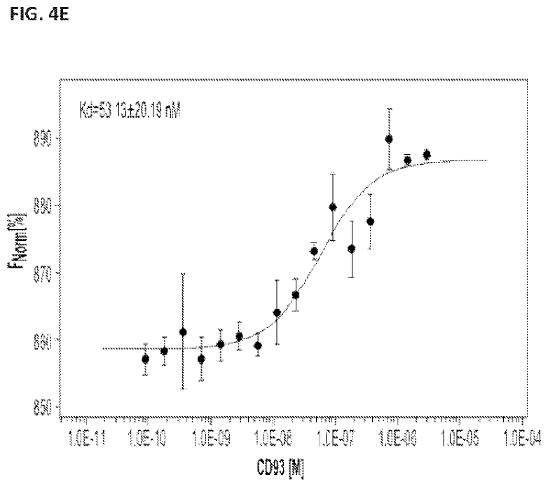

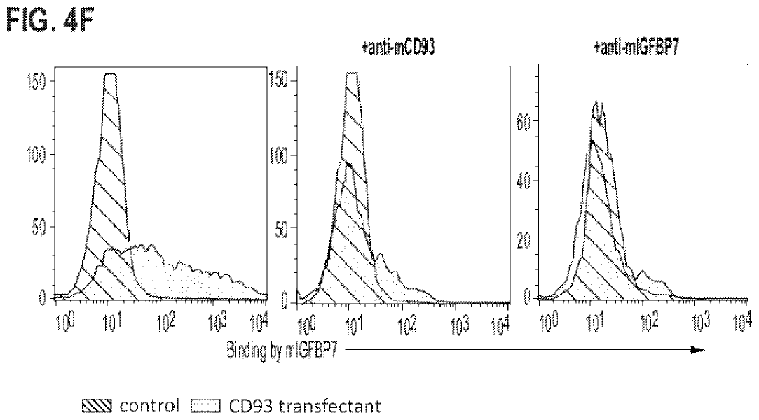

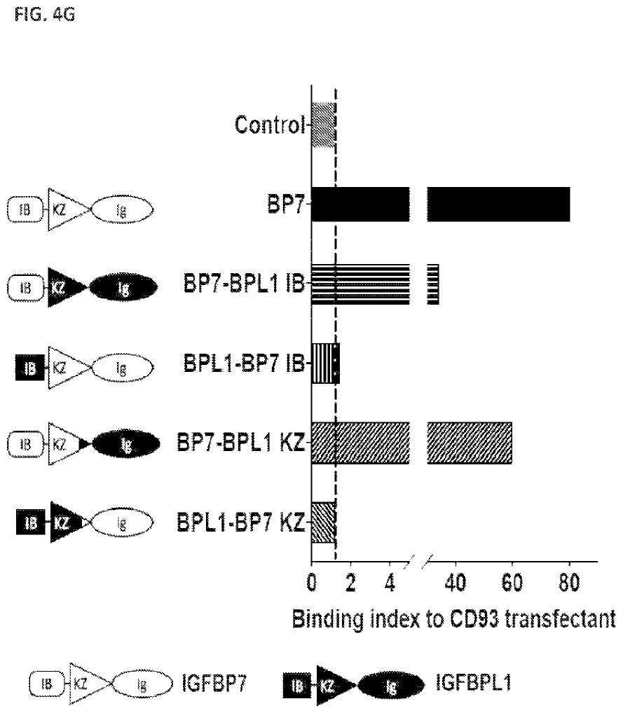

A human genome-scale receptor array (GSRA) was employed to search for counter-receptor of CD93. IGFBP7, a secreted protein of the insulin growth factor binding protein (IGFBP) family, is the only positive hit out of ˜6.600 human transmembrane and secreted proteins in the library (FIG. 4A). The addition of a human CD93 mAb (clone MM01) or IGFBP7 mAb (clone R003) significantly reduced the binding IGFBP7 protein to CD93− transfected 293 cells (FIG. 4B). Recombinant IGFBP7 protein bound HUVEC line positively and the CD93 mAb MM01 completely eliminated this binding activity (FIG. 4C), demonstrating CD93 mediates the binding of IGFBP7 protein to HUVEC line. Furthermore, IGFBP7 could be immunoprecipitated from HUVEC cell lysates with a CD93 mAb, indicating the CD93-IGFBP7 interaction occurs naturally in endothelial cells (ECs) (FIG. 4D). The affinity measurement of the IGFBP7/CD93 interaction by microscale thermophoresis (MST) showed a K_{D} Value at 53.13±20.19 nM (FIG. 4E). The interaction between CD93 and IGFBP7 is also conserved in mouse and this could be blocked by an anti-mouse IGFBP7 mAb (clone 2C6) (FIG. 4F) or an anti-mouse CD93 mAb (clone 7C10) (FIG. 4F) which was used for in vivo functional studies mentioned above. The results suggest that CD93 mAb 7C10 mediates its function in tumor vascular normalization by blocking the IGFBP7/CD93 interaction.

Chimeric proteins of CD93 by replacing its C-lectin domain (˜1-190 aa) with one of its family members were generated. Neither chimeric protein can bind IGFBP7 (data not shown). It suggests the binding site of IGFBP7 on CD93 is the uncharacterized sequence between C-lectin and LAW-like domain (e.g., F182-Y262 of SEQ ID NO: 1).

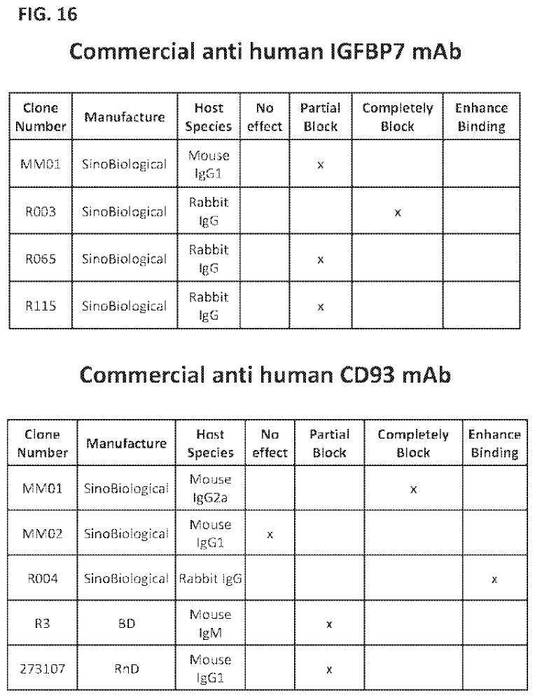

Various commercial anti-human CD93 monoclonal antibodies and anti-human IGFBP7 monoclonal antibodies were tested for their capacity to block the CD93/IGFBP7 interaction. Results were shown in FIG. 16.

### Example 4

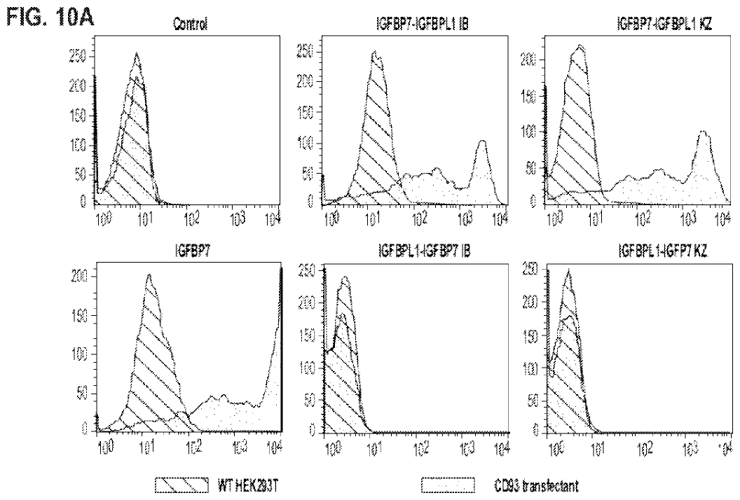

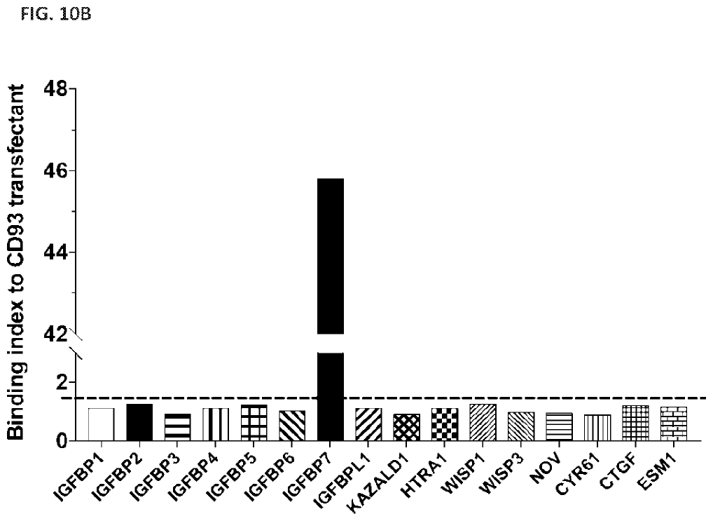

IGFBP7 contains an 16F-binding (IB) domain at its N-terminus, a Kazal-type serine proteinase inhibitor domain (Kazal) in its central region, and an immunoglobulin-like C2-type (IgC2)-domain at its C-terminus (43). To further investigate the binding interaction between IGFBP7 and CD93, a series of chimeric proteins were generated for analysis by replacing each domain of IGFBP7 with a corresponding portion from IGFBPL1 (44), a IGFBP-related protein that does not bind CD93 (FIG. 4G). As expected. IGFBP7, but not IGFBPL1, binds to CD93− 293 cells strongly. Chimeric proteins with the replacement of the IB domain lose the ability to bind CD93+293 cells while the replacements of the Kazal or IgC2 domains have either no or minimal effect. (FIG. 4G and FIG. 10A). To exclude the possibility that other IB domain-containing human protein could also interact with CD93, mouse Fc-tagged fusion proteins were constructed and produced from the majority of the human IB domain-containing genes (n=15). No significant bindings of these recombinant proteins to CD93 were detected except IGFBP7 (FIG. 10B). Therefore, the IB domain on IGFBP7 is highly specific for the interaction with CD93.

### Example 5

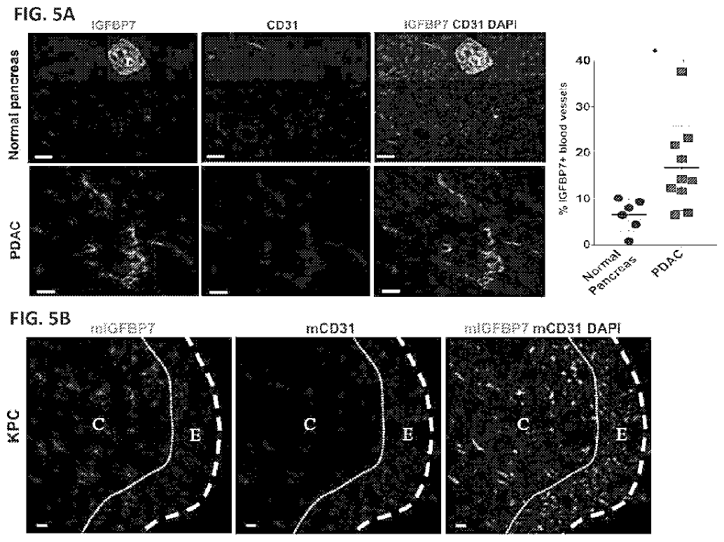

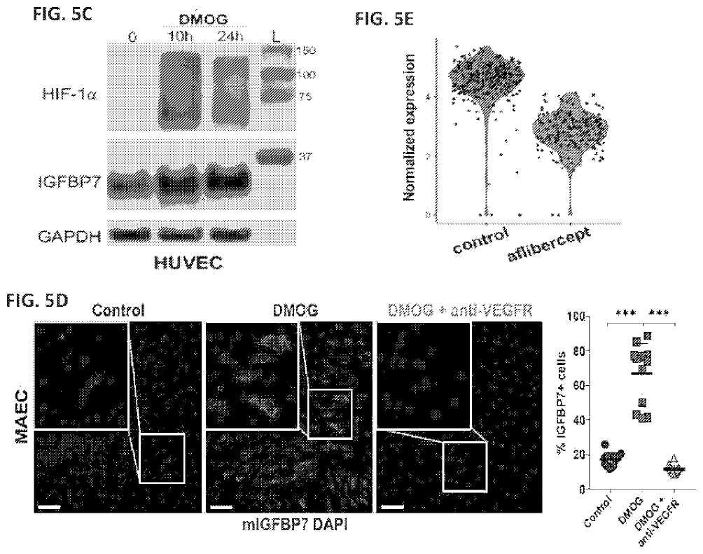

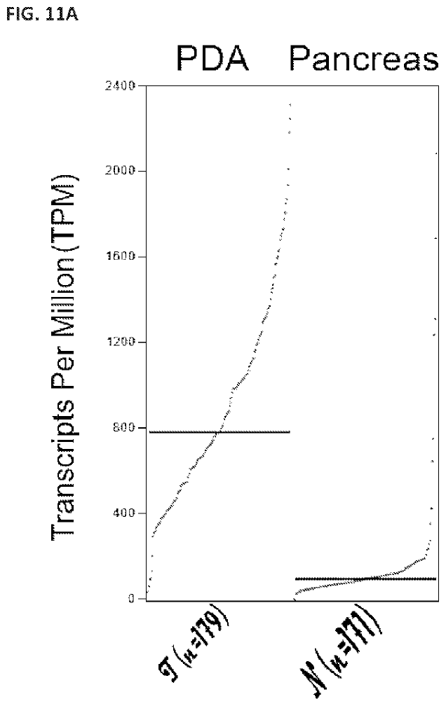

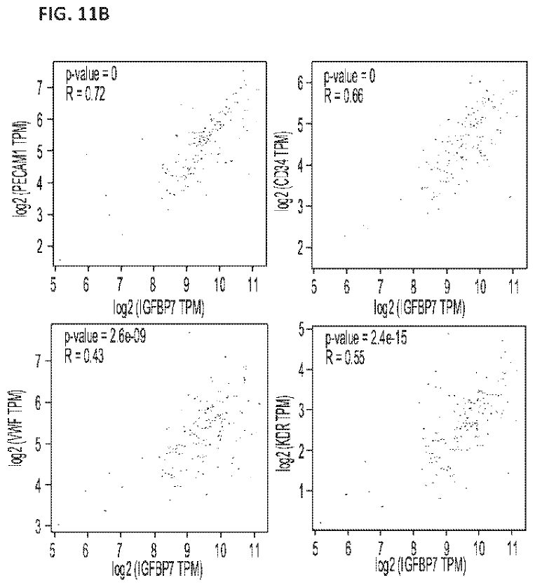

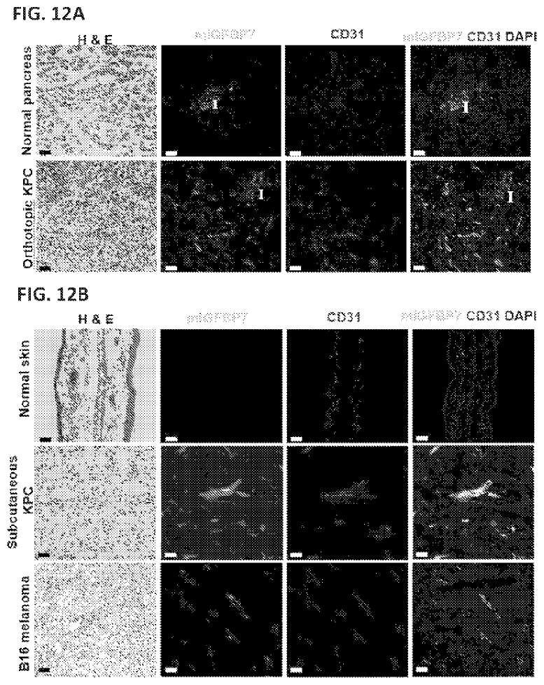

IGFBP7 expression in tissue samples from PDAC patients were analyzed by IF. In adjacent normal pancreas tissues, IGFBP7 protein was mainly present in islet cells, and few blood vessels had detectable IGFBP7 protein. CD31 staining was also scarce in human PDAC tissues. However, there were over twice as many blood vessels which were IGFBP7-positive, compared to adjacent normal pancreas (FIG. 5A). In line with that, analysis of TCGA pancreatic cancer dataset revealed that IGFBP7 was significantly upregulated in human PDAC, compared to normal pancreas (FIG. 11A); the expression IGFBP7 gene is well correlated with EC signature genes, such as PECAM1 (CD31). CD34, and von Willebrand factor (VWF) in PDA, further supporting IGFBP7 as a gene enriched in tumor ECs (FIG. 11B). In mouse cancer tissues, a similar expression pattern of IGFBP7 was observed. In tumor blood vessels, the expression of IGFBP7 was greatly upregulated in orthotopically-implanted KPC (pancreatic adenocarcinoma) tumors, compared to normal pancreas (FIG. 12A). IGFBP7 expression was virtually undetectable in blood vessels of normal mouse skin tissues whereas IGFBP7 was highly expressed in CD31− ECs in subcutaneously implanted mouse KPC and B16 tumors (FIG. 12B).

It was noticed that microvessels within the center of implanted mouse tumor expressed significantly higher level of IGFBP7, compared to those around the edge of the tumor (FIG. 5B), suggesting that IGFBP7 upregulation could be induced by hypoxia within the tumor. To test that. ECs were cultured in dimethyloxalylglycine (DMOG) to mimic hypoxic conditions and examined for IGFBP7 expression by western blot. Indeed, it was found that HUVEC cells cultured in DMOG had increased IIIF-1α levels, accompanied with higher expression of IGFBP7 (FIG. 5C).

Because IGFBP7 gene does not have a consensus hypoxia response element (HRE, the 5′-RCGTG-3′ motif) (47) in the promoter region, its upregulation in ECs may not be directly triggered by hypoxia. It was hypothesized that hypoxia-induced VEGF, a strong inducer of IGFBP7 in ECs (48), could be responsible for IGFBP7 upregulation. This hypothesis was tested in mouse endothelial cells. Similar to HUVEC cells. IGFBP7 expression could be upregulated in mouse ECs in the presence of DWOG to mimic hypoxic condition. Inclusion of a VEGFR blocking mAb to the culture completely prevented hypoxia-induced IGFBP7 expression in mouse ECs (FIG. 5D), suggesting that hypoxia-induced IGFBP7 is fully dependent on VEGF signaling in this system. Interestingly, analysis of the RNA-Seq data (GSE110501) from a xenograft colon cancer mouse model (49) indicated that IGFBP7 was also significantly inhibited in tumor ECs by aflibercept, a VEGF inhibitor (FIG. 5E). Taken together, these results support that IGFBP7 is a hypoxia-induced ECM protein in tumor-associated vasculature by VEGF signaling.

### Example 6

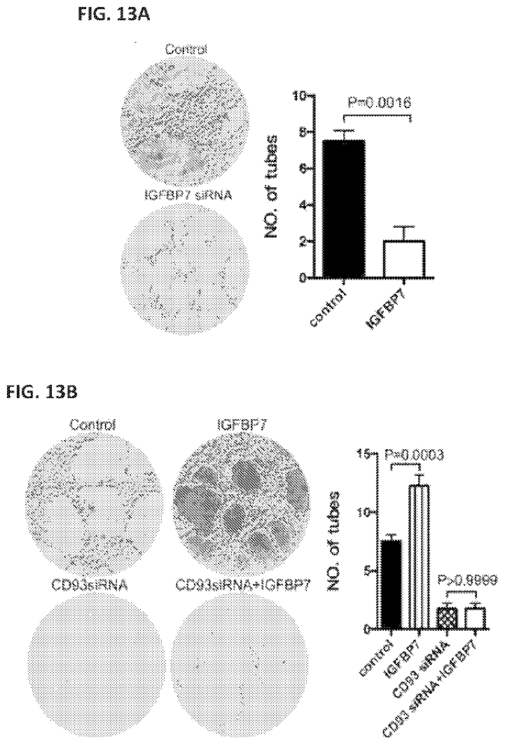

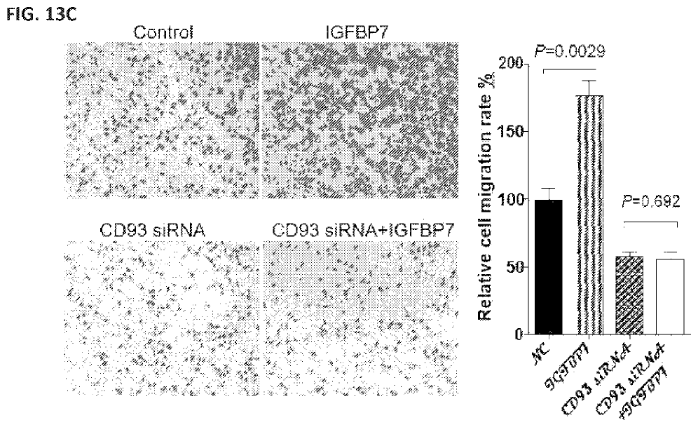

IGFBP7 protein was constitutively expressed in HUVEC cells and further upregulated by DMOG, accompanied by the induction of HIF-1α (FIG. 5C). The knockdown of IGFBP7 gene expression significantly inhibited the tube formation in HUVEC cells (FIG. 13A). To determine whether IGFBP7 mediates vascular angiogenesis via CD93, HUVEC cells were transfected with CD93 siRNA to knockdown CD93 expression as an in vitro model to test the effect of IGFBP7 protein. The addition of exogenous IGFBP7 protein increased wild type HUVEC cell tube formation and proliferation. However, in the CD93-knowndowned HUVEC cells. IGFBP7 protein lost its effect on the tube formation or EC migration in a transwell migration assay (FIGS. 13B and 13C). These studies indicate that CD93 mediates the proangiogenic effect of IGFBP7 protein on ECs.

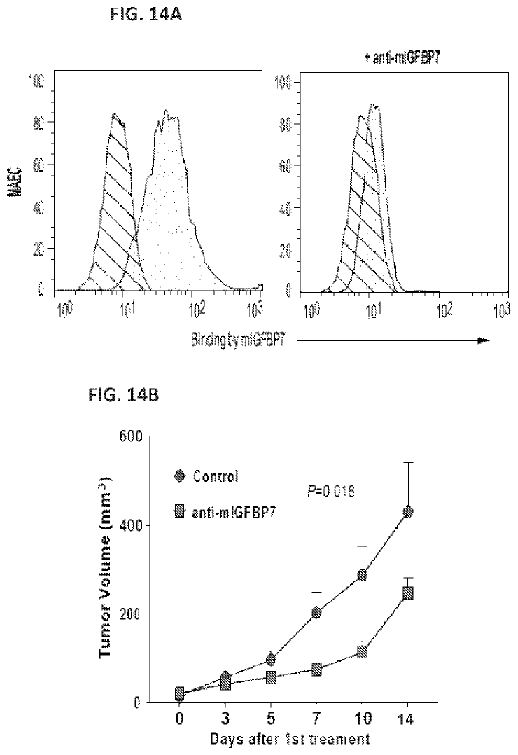

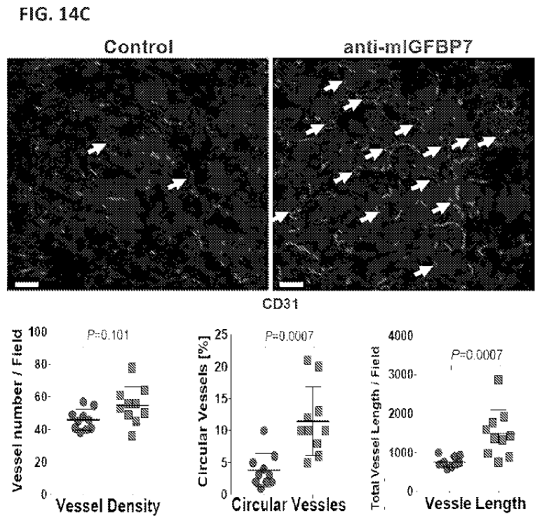

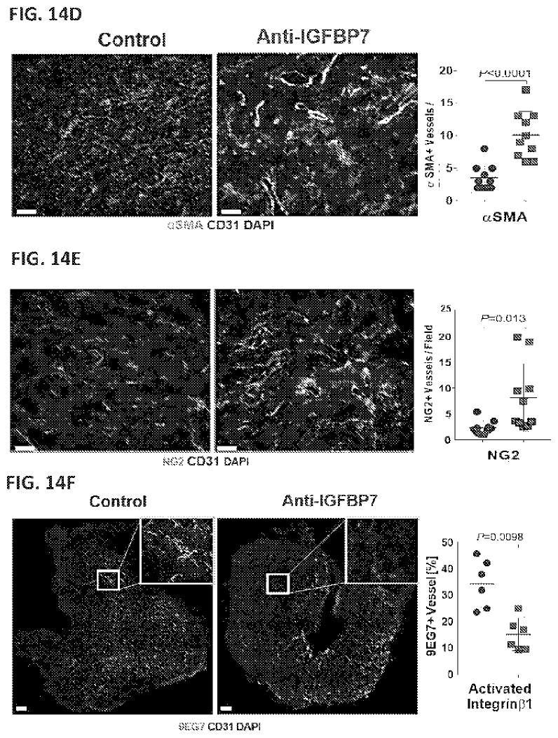

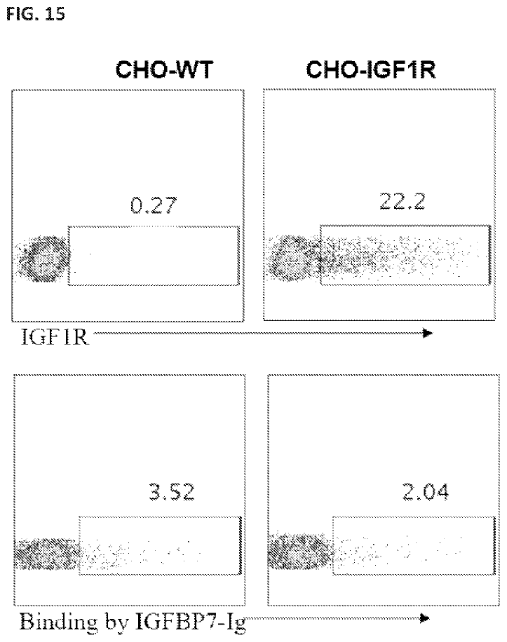

An IGFBP7 mAb (clone 2C6. FIG. 14A), which blocks the binding of IGFBP7 to CD93, was utilized to test its effect on tumor growth and tumor vascular maturation in vivo. Administration of 2C6 significantly inhibited KPC tumor growth as described above by over 40% relative to the control (FIG. 14B). IF staining of tumor tissues revealed that blockade of the IGFBP7/CD93 interaction by 2C6 greatly increased circular vessels and length of tumor microvessels but did not affect the density of CD31− tumor vessels (FIG. 14C). Similar to the effect on vascular maturation by the CD93 mAb, IGFBP7 mAb improved the coverage of NG2− pericytes alongside tumor vessels (FIG. 14D), and increased α-SMA coverage over tumor vessels (FIG. 14E). Tumor tissues from mice treated with 2C6 mAb displayed a clear reduction of β1 integrin activation by over 50% (FIG. 14F), further supporting that anti-IGFBP7 affects integrins to normalize tumor vessels (51). These results support that blockade of the IGFBP7/CD93 interaction promotes vascular normalization and attenuates tumor growth.

Additionally, high dosage of IGFBP7 and CD93 mAbs (15 mg/kg, or 300 μg) did not reduce tumor vascular density in vivo. The results suggest that main effect of altered CD93/IGFBP7 in the TME is on vascular abnormality but not increased angiogenesis. This indicates that the IGFBP7/CD93 axis could be a better therapeutic target for vascular normalization. Both IGFBP7 and CD93 are selectively upregulated on tumor blood vessels of mouse and human tumors. These limited expression patterns are contrary to broad display of VEGFR-1, -2 and -3 in microvessel in normal tissues.

### Example 7

With the profound effects of the CD93/IGFBP7 interaction in abnormalities of tumor angiogenesis, it was further tested whether blockade of this interaction by mAb could improve tumor perfusion so as to promote drug delivery as a result of vascular normalization. In the KPC model, the delivery efficacy of doxorubicin, an anthracycline chemotherapeutic with intrinsic autofluorescence was tested. Mice were i.v. infected with doxorubicin 20 min before sacrificing. At the same time, mice were treated with pimonidazole as a hypoxyprobe to evaluate possible changes in tumor hypoxia. Greater penetration of doxorubicin into tumors was observed in CD93 mAb-treated mice; in the meantime, hypoxia was also significantly reduced in tumors (FIG. 6A). It was also evaluated the antitumor effect of anti-CD93 in B16 tumor model with 5-fluorouracil (5-FU) treatment. Mice were s.c. implanted with B16 melanoma and started with the treatment of CD93 mAb twice a week, followed with two doses of 5-FU once tumor became palpable. As expected, the treatment of CD93 mAb or 5-FU alone only modestly inhibited tumor growth, and eventually tumor outgrew in both groups. The combinatory treatment of 5-FU and CD93 mAb was able to dramatically inhibit tumor growth (FIG. 6B) and extended survival of a significant portion (about 40%) of mice over 20 days (FIG. 6C). Tissue staining indicated that CD93 blockade enhanced 5-FU-induced suppression of tumor proliferation, based on Ki-67 staining of implanted B16 melanoma (FIG. 6D). Taken together, these experiments demonstrate that blockade of the CD93/IGFBP7 interaction reduces hypoxia, promotes drug delivery, and therefore facilitates chemotherapy in cancer.

### Example 8

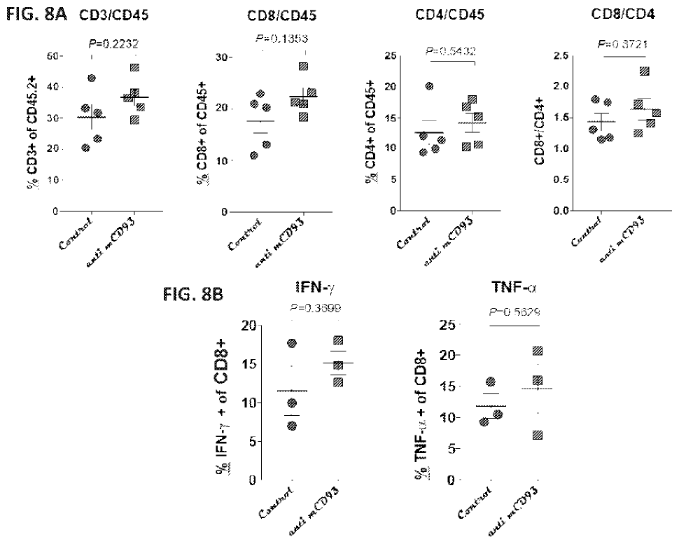

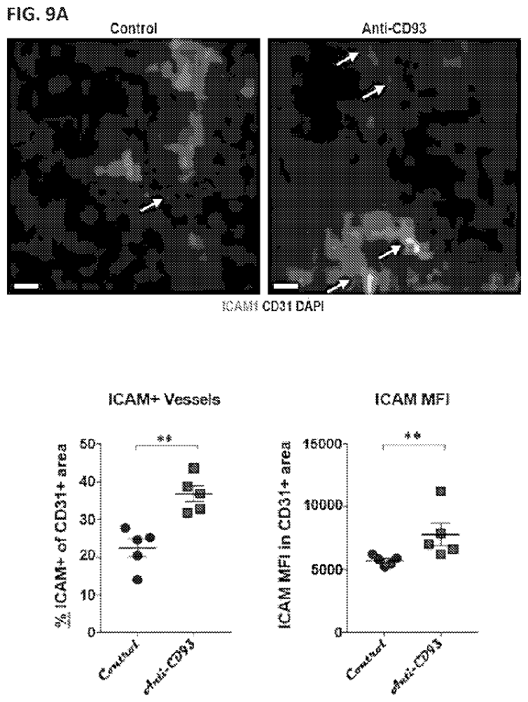

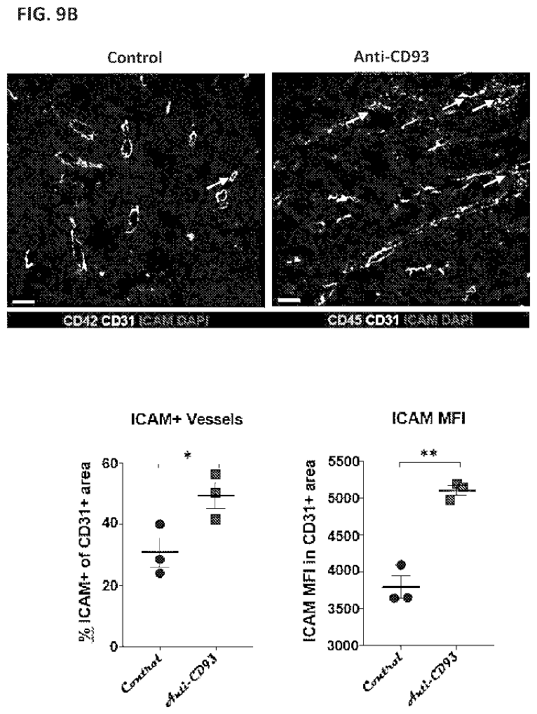

Normalization of tumor vasculature could enhance immune cell trafficking into the tumors, which may be due to upregulated adhesion molecules (16, 40, 41). It was found that anti-CD93 treatment increased ICAM1 expression on tumor blood vessels in both s.c. KPC and B16 tumor models (FIGS. 9A and 9B). In line with that, IF staining of CD3 revealed ˜3-fold increases in TILs in KPC tumor tissues from anti-CD93 treated mice, compared to those from the controls in day 8 and 15 (FIGS. 3A and 3B). Further analysis of TIL compositions by flow cytometry reveals that anti-CD93 greatly increased the percentage and absolute number of CD45− leukocytes in tumors: ˜3-fold more CD4+ and CD8− T cells in CD93 mAb-treated tumors than the controls (FIGS. 3C and 3D). Anti-CD93 did not alter the proportions of CD8+ or CD4− TIL subset within the CD45+ hematopoietic cell compartments (FIG. 8A), as well as functions as shown by similar levels of IFN-gamma and TNF-alpha of TILs (FIG. 8B). However, anti-CD93 significantly reduced the percentages of myeloid-derived suppressor cells (MDSCs) within tumors (FIG. 3E), further supporting a favorable inflammatory TME. A similar effect of anti-CD93 on promoting TILs in B16 melanoma was observed, though there were generally fewer TILs within tumors in this model (FIG. 3F). Taken together, these results support that blockade of CD93/IGFBP7 interaction conditions an inflammatory TME by improving T cell infiltration.

### Example 9

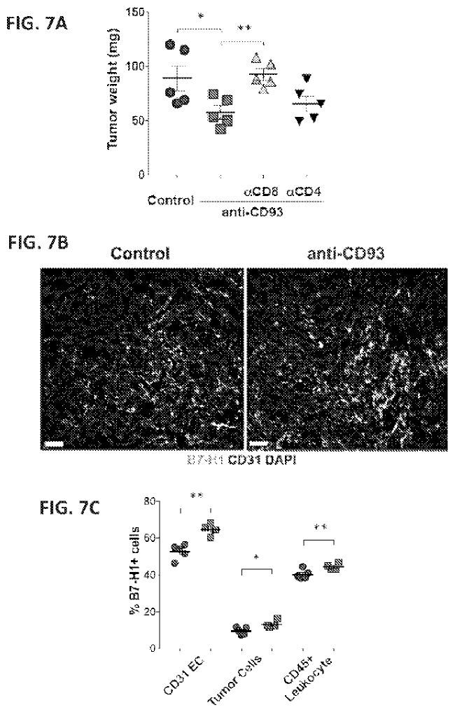

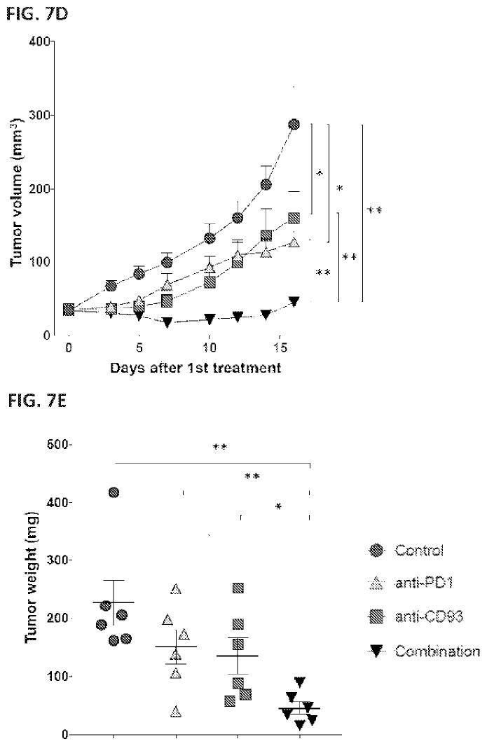

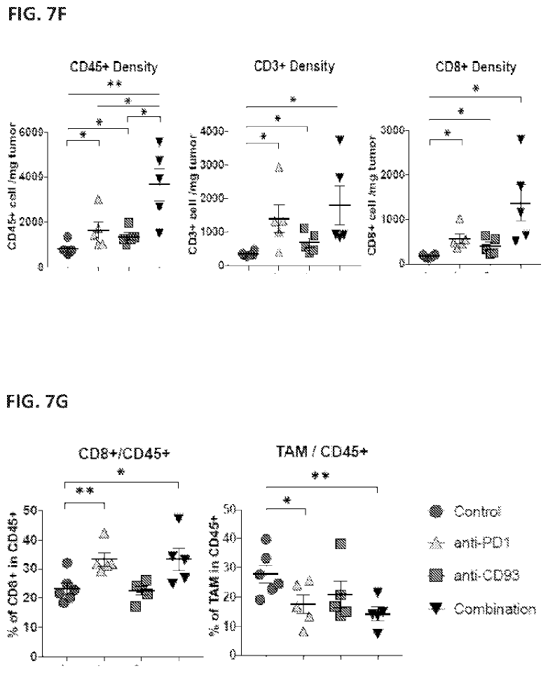

Whether blockade of the CD93/IGFBP7 could facilitate cancer immunotherapy based on immune normalization of tumor microenvironment was tested. It was first determined whether the effect of anti-CD93 on inhibiting tumor growth is dependent on T cell-mediated immune responses. Depleting CD8+ cells by mAb at the beginning of anti-CD3 treatment completely diminished the antitumor effect, while depletion of CD4+ T cells had only a small effect (FIG. 7A), supporting a major role of CD8− cells in anti-CD93 mediated tumor suppression in this model.

It was hypothesized that B7-H1 induction may be responsible for a limited antitumor effect by anti-CD93. Indeed, an upregulation of B7-H1 expression on tumor tissues was observed upon anti-CD93 treatment (FIG. 7B). In addition to increased B7-H1 expression in CD31+ tumor ECs, significant increases of B7-H1 expression was also observed in both tumor cells and CD45+ leukocytes in anti-CD93-treated tumors than the controls (FIG. 7C). Therefore, upregulation of B7-H1 in the TME by anti-CD93 may limit antitumor immunity and these findings justify a combined therapy of anti-CD93 with anti-PD-1/PD-L1 therapy and this possibility was subsequently tested in the KPC model. While the treatment by anti-CD93 or anti-PD-1 mAb alone partially retarded turner growth, a combination by anti-CD93/PD-1 mAb profoundly inhibited tumor growth in this model (FIG. 7D). As a result, tumor weights in the combination group were reduced to only about 20% of the control group (FIG. 7E). Consistent with a better antitumor effect analysis of immune cells within the tumors with the combinatory therapy indicated a vastly increase of absolute numbers of both CD8+ and CD4− T cells (FIG. 7F). Accompanied with that, the proportion of CD8+ T cells was significantly increased while tumor-associated macrophages (TAMs) were greatly reduced in the combinatory group (FIG. 7G). These results indicate that blockade of the CD93/IGFBP7 could normalize tumor vasculature which could amplify the effect of anti-PD-1/PD-L1 cancer immunotherapy.

### Example 10

This Example demonstrates that CD93 on nonhematopoietic cells mediates the antitumor immunity shown by anti-CD93. It was found that anti-CD93 mAb accumulated on tumor vasculature of B16 tumors upon injection (FIG. 17A). In addition to ECs. CD93 is known to be expressed on several hematopoietic cell types, including monocytes, macrophages, and immature B cells (71). To fully reveal the cellular source of CD93 responsible for the antitumor effect of anti-CD93 treatment. CD93 chimeric mice were made by reconstituting lethally-irradiated WT 136 mice with hone marrow (BM) from WT or CD93KO mice. As expected, the treatment of anti-CD93 inhibited tumor growth in chimeric mice, regardless of the source of BM (FIG. 17B). As ECs are the only cellular source for CD93 in nonhematopoietic cells, the results confirmed that anti-CD93 is a blocking mAb to target tumor vasculature.

### Example 11

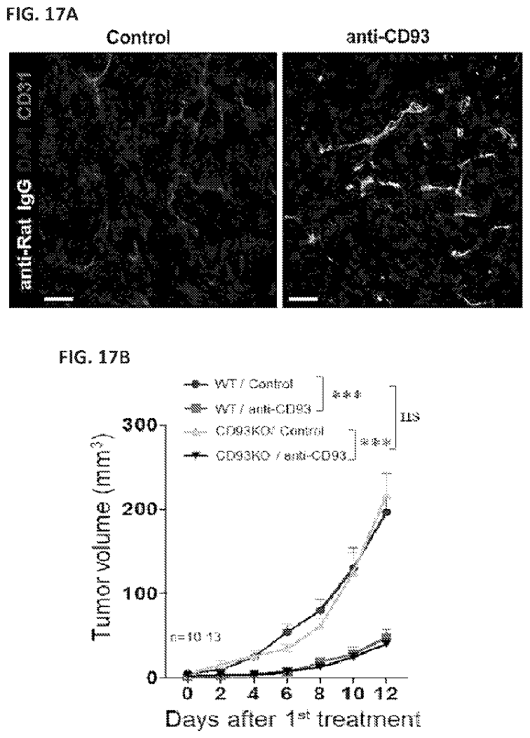

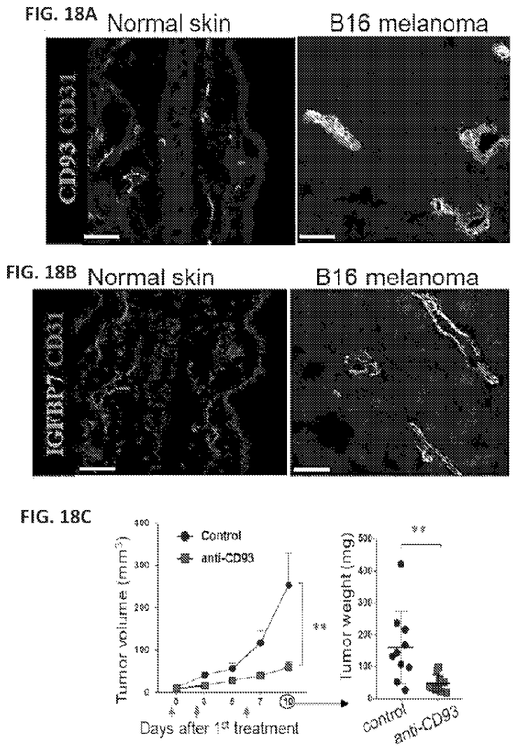

This Example demonstrates that CD93 blockade inhibits B16 melanoma tumor growth. CD93 overexpression in tumor vasculatures has been observed in many solid tumors (32-34). Similarly. CD93 (FIG. 18A) and IGFBP7 (FIG. 18B) in tumor vasculature are both markedly upregulated in subcutaneous B16 melanoma. When tumor-bearing mice were treated with the blocking mCD93 mAb (Clone 7C10), CD93 blockade significantly inhibited tumor growth and reduced tumor weight in B16 tumors (FIG. 18C). The treatment with the Fab of anti-CD93 was still effective in inhibiting B16 tumor growth, excluding the possibility of Fe-mediated depletion (data not shown). These data are consistent with retarded tumor growth seen in CD93−/− mice.

### Example 12

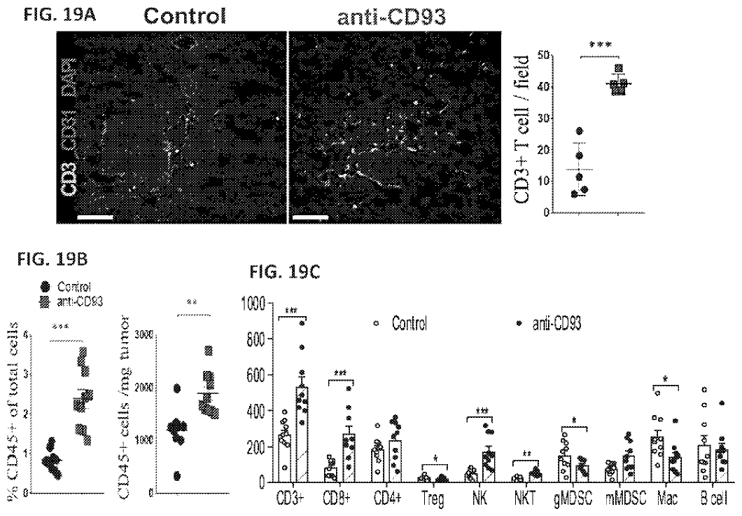

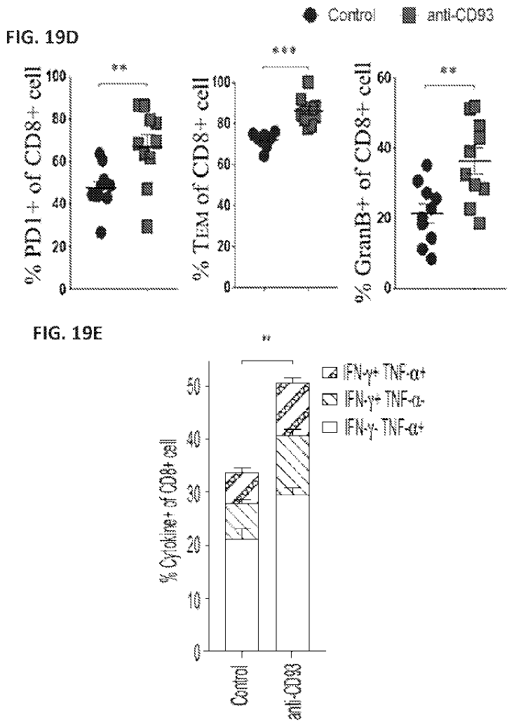

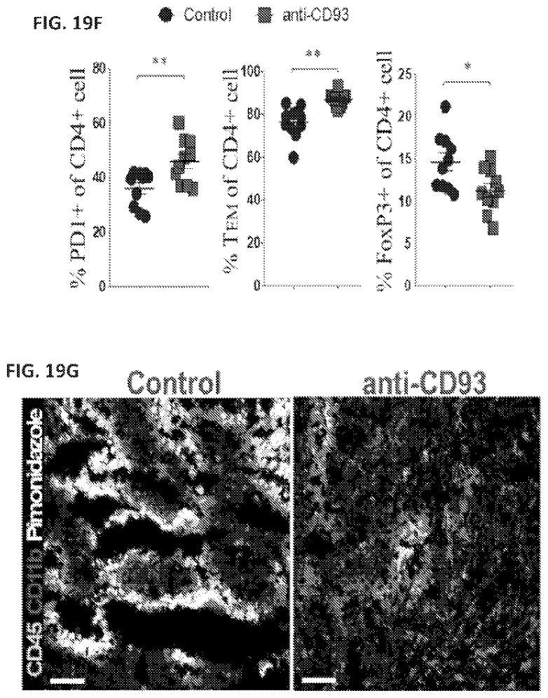

This Example demonstrates that CD93 blockade greatly increases T cell infiltration and function in mouse melanoma. Normalization of tumor vasculature enhances immune cell trafficking into the tumors (16, 74). It was found that anti-CD93 treatment led to about three fold increase of CD3+ TILs in B16 tumors (FIG. 19A). Flow cytometry analysis revealed that anti-CD93 greatly increased both the percentage and density of CD45− immune cells in the tumor (FIG. 19B). Detailed analysis of immune cell composition indicated that NK and T cells, particularly CD8− T cells, are the major cell types increased within anti-CD93− treated B16 tumors (FIG. 19C). Anti-CD93 significantly increased the percentages of effector memory T cells (TEM) in CD8− T cell subsets, as further confirmed by increased PD1 and Granzyme B expressions (FIG. 19D); consistently, CD8− TILs within CD93− treated tumors produced significantly more effector cytokines including IFN-γ and TNF (FIG. 19E). Though CD93 blockade did not affect the density of CD4− TILs, there were proportionally more effector T cells (TEM and PD1-positive) and fewer Treg cells in anti-CD93− treated tumors (FIG. 19F). The analysis also revealed that man immunosuppressive cells, including Treg granulocytic myeloid-derived suppressor cells (gMDSC) and tumor-associated macrophages (Mac), were significantly reduced in tumors treated with anti-CD93 (FIG. 19C). MDSCs and macrophages (CD11b+) preferentially localized to hypoxic areas: since MDSCs and macrophages do not express CD93 themselves, their reductions in anti-CD93-treated tumors could be caused by reduced hypoxia. (FIG. 19G) Taken together, the results support that blockade of the CD93 pathway conditions an immune-favorable TME in B16 melanoma.

### Example 13

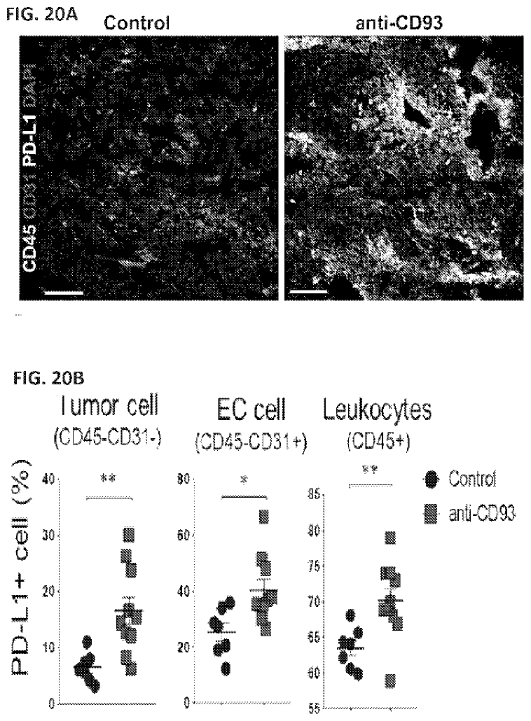

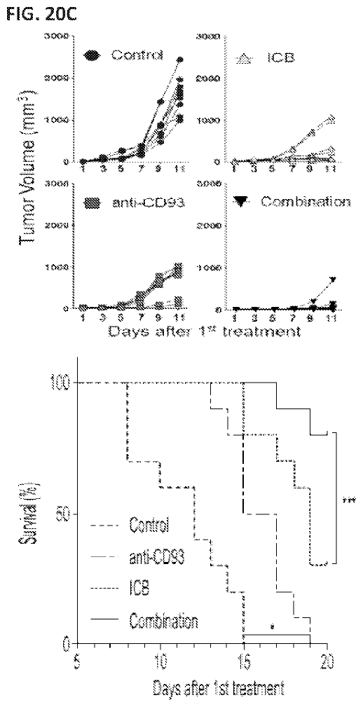

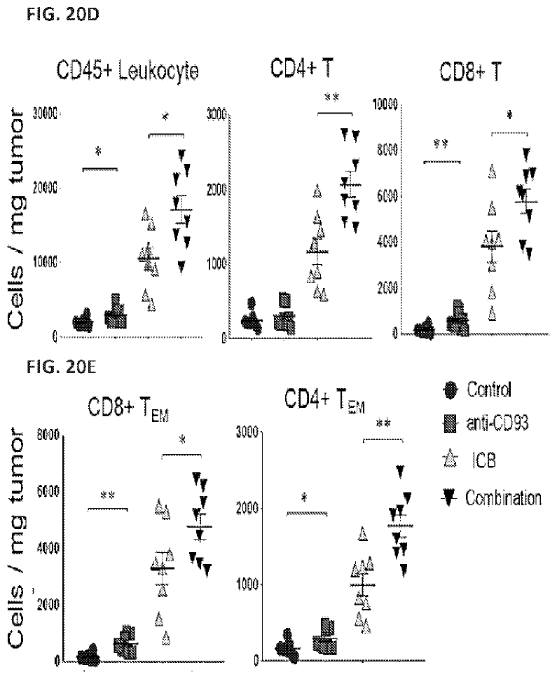

This Example demonstrates that CD93 blockade sensitizes B16 melanoma to immunotherapy. PD-L1 is often upregulated in tumor tissues in response to IFN-γ as a result of increased TILs (52). Indeed, an upregulation of PD-L1 expression was observed on tumor tissues upon anti-CD93 treatment (FIG. 20A). In addition to CD31− ECs, a significant increase of PD-L1 expression was observed in both tumor cells and CD45+ leukocytes by anti-CD93 (FIG. 20B). Furthermore, PD1-positive TILs were more abundant in B16 tumors under anti-CD93 treatment (FIGS. 19E and 19G). This observed upregulation of the PD1/PD-L1 pathway in the TME may limit antitumor immunity by anti-CD93. In the B16 melanoma model, the treatment of anti-CD93 or ICB (PD1 plus CTLA4 blocking mAbs) alone modestly retarded tumor growth. However, combination of anti-CD93/ICB profoundly inhibited tumor growth in this model; over 80% of mice in the combination group survived over 20 days, while all mice of the control group died before 15 days (FIG. 20C). Consistent with a better antitumor effect, analysis of immune cells within the tumors of the combinatory therapy indicated vastly increased numbers of CD45− immune cells, including both CD4− and CD8+ T cells (FIG. 20D). Concurrently, the numbers of T cells with effector memory phenotype (T_{EM} CD44^{hi}CD62L−) were significantly increased in both CD4− and CD8+ T cells in the combinatory group (FIG. 20E). Together, the results support that blockade of CD93 signaling sensitizes tumors to ICB therapy.

### Example 14

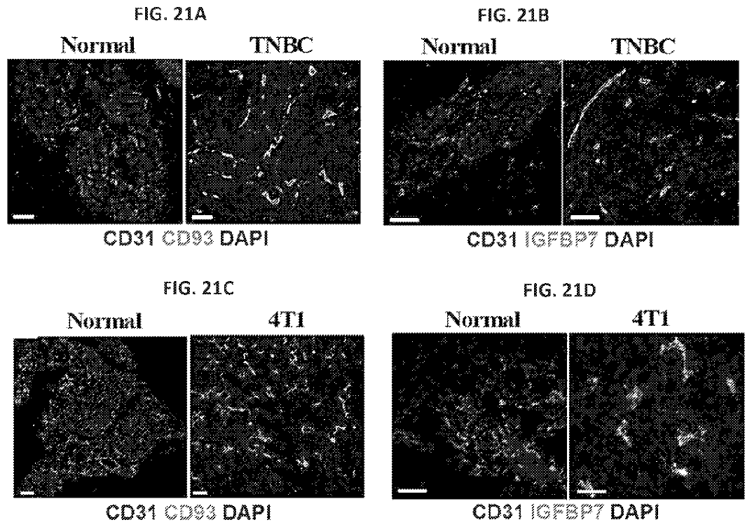

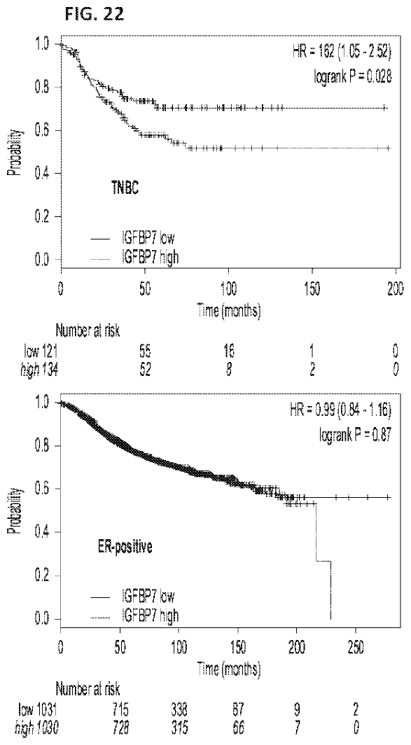

This Example demonstrates that expression of the IGFBP7/CD93 pathway is upregulated in TNBC vasculature. CD93 is one of the top genes in a previously reported human primary tumor angiogenesis gene signature (45), and CD93 overexpression in tumor vasculatures has been observed in main solid tumors (30, 74-76). It was found that CD93 was clearly upregulated on blood vessels within human TNBCs (n=5), compared to those in adjacent normal breast tissues (FIG. 21A). IGFBP7 protein was barely detectible in blood vessels of adjacent normal breast tissue, however, its expression in human TNBC vasculatures was markedly increased (FIG. 21B). Similarly, in an orthotopic 4T1 mouse beast tumor model, the expressions of CD93 (FIG. 21C) and IGFBP7 (FIG. 21D) in tumor vasculature were both drastically upregulated. To assess the clinical relevance of IGFBP7 in BCs, the TCGA breast cancer dataset was analyzed. Interestingly, high IGFBP7 is associated with poor prognosis in TNBC, but not in ER-positive breast cancer (FIG. 22).

### Example 15

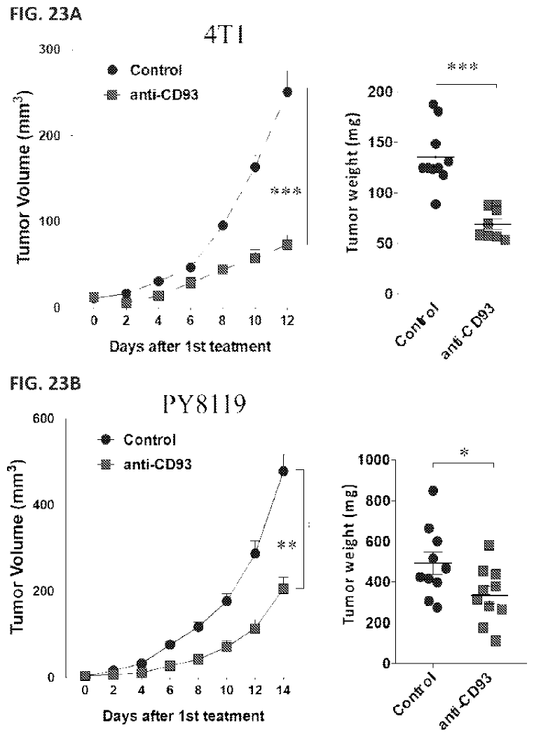

This Example demonstrates that blockade of the IGFBP7/CD93 interaction inhibits TNBC tumor growth in vivo. 4T1 tumor-hearing mice were treated with the blocking mCD93 mAb (Clone 7C10) when 4T1 tumors became palpable. Tumor growth curves indicated that administration of anti-CD93 blocking mAb significantly inhibited tumor growth and thus reduced tumor weight (FIG. 23A). Similarly, the same CD93 blocking mAb had a comparable antitumor effect on orthotopically-implanted PY8119 (FIG. 23B), another mouse TNBC model.

### Example 16

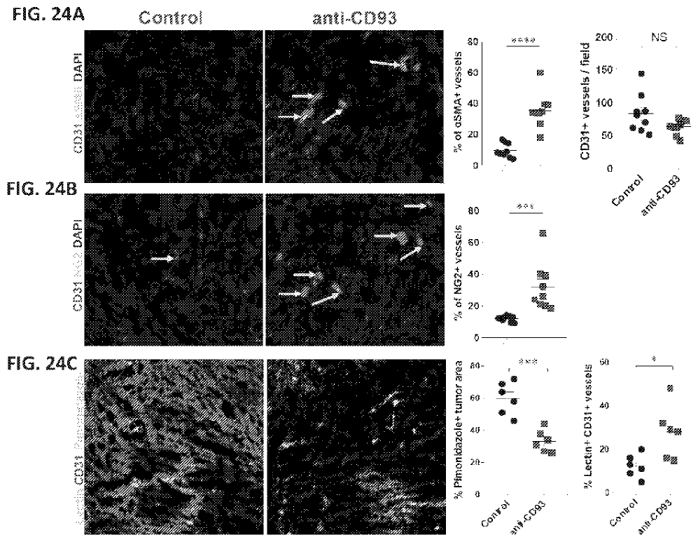

This Example demonstrates that CD93 blockade promotes vascular maturation to improve perfusion in TNBC. Blockade of the IGFBP7/CD93 interaction by CD93 mAb did not affect vessel density (FIG. 24A). The effect of CD93 mAb on tumor vascular normalization was confirmed by increased α-SMA staining on tumor vascular vessels (FIG. 24A) and pericyte coverage (NG2+ vessels. FIG. 24B). A similar result was found for anti-CD93 on vascular maturation in PY8119 tumor model (data not shown). CD93 blockade increased tumor perfusion, as there were over two-fold increase of FITC-lectin-positive blood vessels in tumors treated with CD93 mAb; accompanied with that, there were significantly less hypoxic area (pimonidazole+) in 4T1 tumors with anti-CD93 treatment (FIG. 24C).

### Example 17

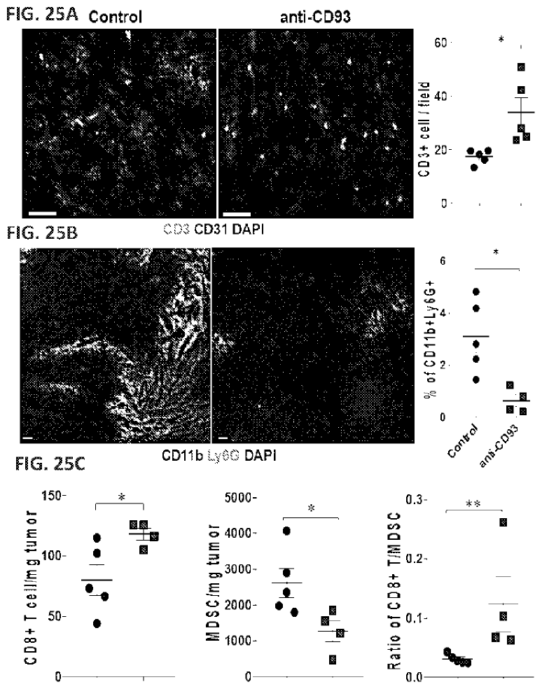

This Example demonstrates that increased TILs and reduced MDSCs in 4T1 upon CD93 blockade. Upon two weeks of antibody treatment, infiltrating immune cells were examined in 4T1 tumors by IF staining. It was found that there were significantly more CD3+ T cells in tumors treated with CD93 mAb (FIG. 25A). The CD11b+Ly6G+ MDSCs are abundant in 4T1 tumors. Interesting, the treatment of anti-CD93 greatly reduced its number in tumors (FIG. 25B). The IF results of tumor cell suspension were further confirmed via FACS analysis (FIG. 25C). Thus CD93 blockade can create a favorable TME for immunotherapy in TNBC.

### Example 18

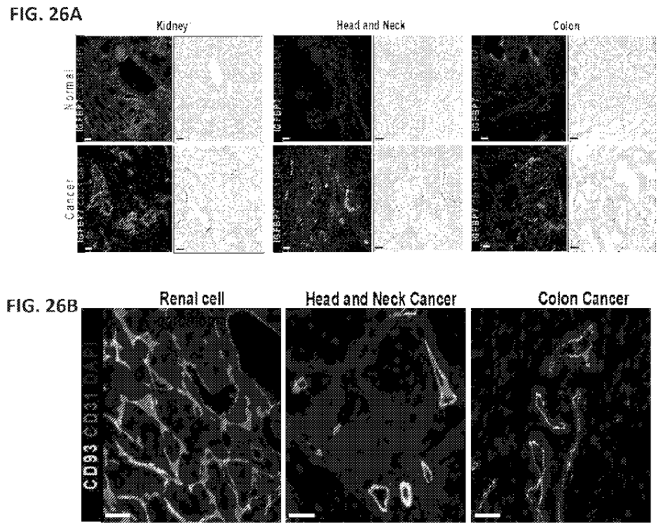

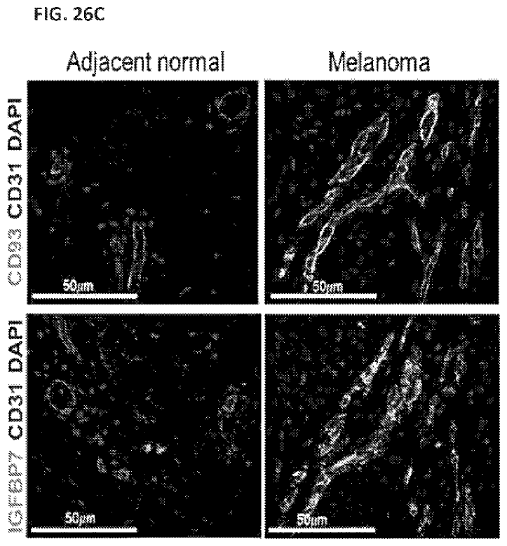

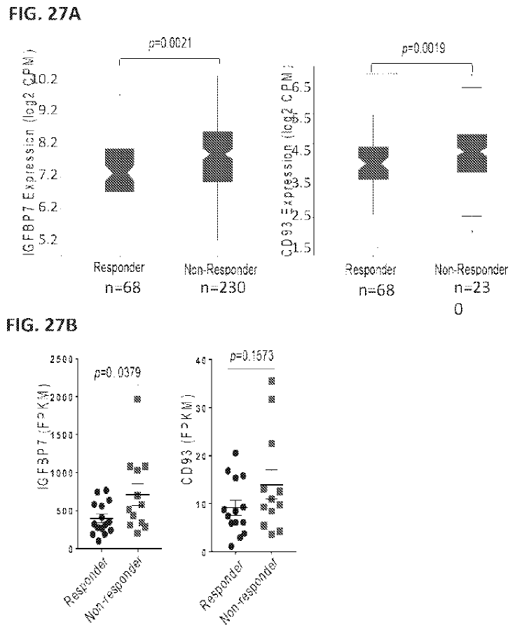

This Example demonstrates that IGFBP7 and CD93 are upregulated in vasculatures within human cancers. The expressions of IGFBP7 are upregulated in human cancers, compared to adjacent normal tissues (FIG. 26A). CD93 expression in human cancers is mainly present on tumor vasculature, based on immunofluorescent staining (FIG. 26B). Both CD93 and IGFBP7 are upregulated in blood vessels within human melanoma (FIG. 26C).

### Example 19

This Example demonstrates that enrichment of the IGFBP7/CD93 pathway in human cancers resistant to anti-PD therapy. Tumor vascular dysfunction limits antitumor immunity and poses a great threat to immunotherapy (19). Gene expressions of IGFBP7 and CD93 was examined in cancer patients under anti-PD therapy. In a phase II trial of patients with metastatic urothelial cancer receiving atezolizumab (anti-PD-L1 mAb) treatment (77), baseline levels of IGFBP7 and CD93 expressions were both significantly higher in tumor tissues from non-responders compared to those from responders (FIG. 27A). Consistently, in a small cohort of metastatic melanoma patients under anti-PD1 treatment (78), baseline IGFBP7 levels tended to be lower in patients who were responsive to anti-PD1 therapy compared to patients who did not benefit (FIG. 27B). A trend toward increased mean CD93 expression in non-responders was observed, although this association did not reach statistical significance (FIG. 27B). In summary, the IGFBP7/CD93 pathway in the TME may contribute to cancer resistance of anti-PD therapy in clinic.

### Example 20

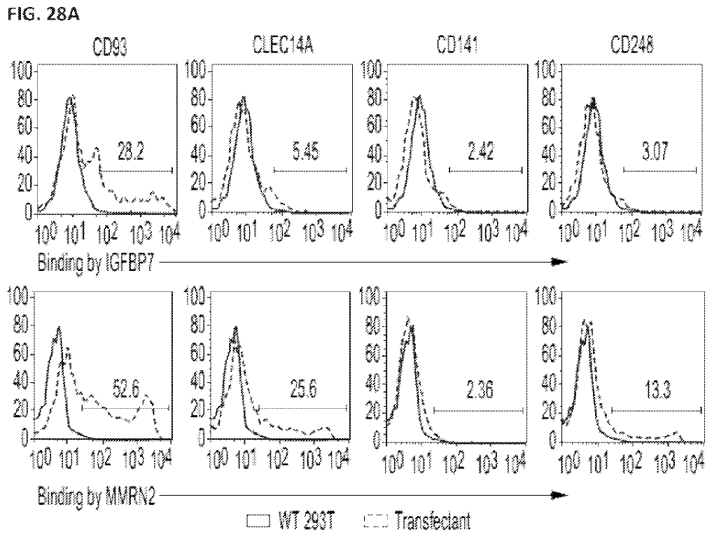

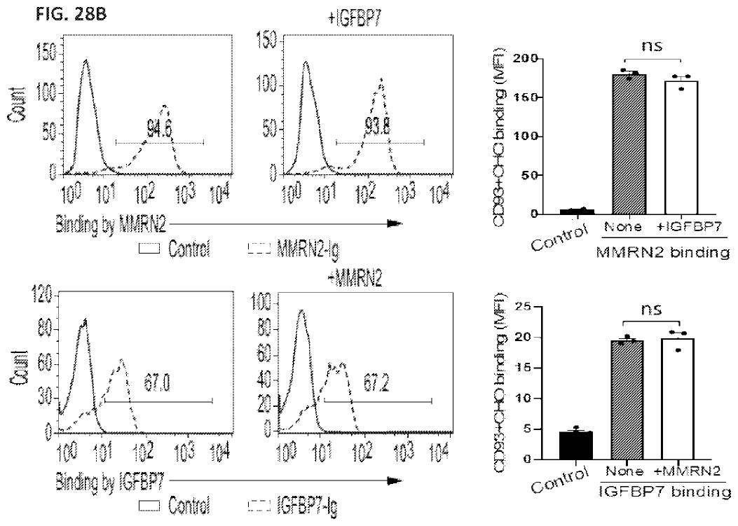

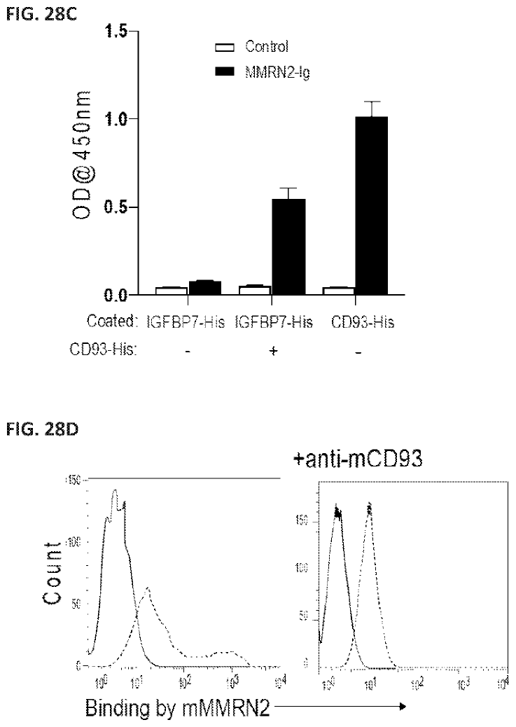

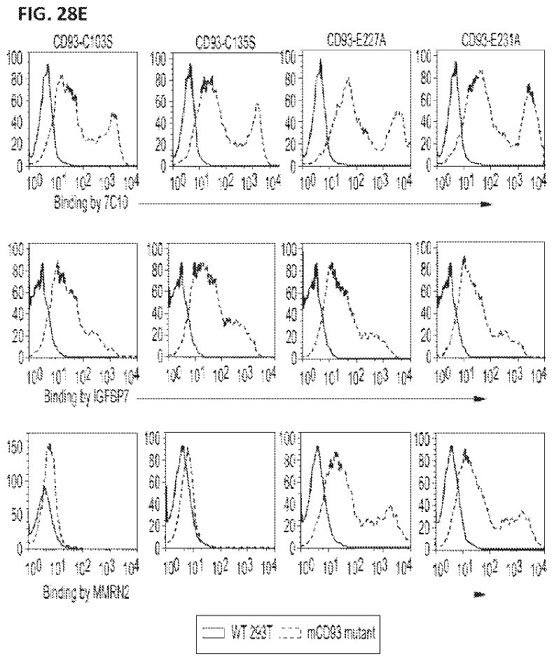

This Example demonstrates that IGFBP7 and MMRN2 bind to different motif of CD93. MMRN2, an ECM protein which happens not be present in the GSRA library (42), is another known ligand for CD93. Besides CD93, MMRN2 also interacts with CLEC14A and CD248, two additional group 14 C-type lectin members; in contrast to MMRN2. IGFBP7 only bound to CD93 but not any other C-type Lectin molecule (FIG. 28A). MMRN2 and IGFBP7 did not compete each other for CD93 binding as the addition of IGFBP7 did not interfere with the CD93 binding by MMRN2, and vice versa (FIG. 28B). Supporting that, in an ELISA assay, the pre-incubation IGFBP7-coated wells with CD93 protein led to MMRN2 binding (FIG. 28C), this suggested that CD93 can bind to its two ligands at the same time to form a protein complex together. It was also found that the anti-mouse CD93 (clone 7C10) used for in vivo studies also blocked the interaction between CD93 and MMRN2 (FIG. 28D). When the bindings of these two ligands to several mouse CD93 with point mutations was examined, it was found that two of CD93 mutants (C103S and C135S), which lose the binding to MMRN2, bound to IGFBP7 greatly (FIG. 28E). All these supported that IGFBP7 and MMRN2 bind to different positions on CD93.

Below are the methods and materials used in the Examples.

### Cell Lines, Fusion Proteins and Antibodies

KPC cell was derived from KrasLSLG12D/; Trp53R172H; Pdx1-Cre (KPC) transgenic mice. Human IGFBP7 (Fc-tang) and Mouse IGFBP7 (Fc-tag) were purchased from Sino Biological. Rat anti-mouse CD93 mAb (clone 7C10) was generated from a hybridoma derived from the fusion of SP2 myeloma with B cells from a rat immunised with mouse CD93-Ig. Hamster anti-mouse IGFBP7 mAbs (clone 2C6, 6F1) were generated from hybridomas derived from the fusion of SP2 myeloma with B cells from Armenian hamster immunised with mouse IGFBP7-Ig. Hybridomas were adapted and cultured in Hybridoma-serum-free media (Life Technologies). Antibodies in supernatant were purified by HiTrap protein G affinity column (GE Healthcare). Anti-mouse VEGFR-2 (clone DC101) was purchased from BioXcell. Anti-human IGFBP7 mAb (R003, SinoBiological) and anti-human CD93(MM01, SinoBiological) were used to block human IGFBP7-CD93 interaction. Commercial antibodies, if not listed, were purchased from Biolegend.

### IGFBP7 Chimeras and CD93-F238L Mutant

The IGFBP7-IGFBPL1 chimeras were generated by two-step PCR. The chimeric proteins share the similar structure and contain the domains from IGFBP7 and IGFBPL1 were interchanged at different cut sites. The supernatants were collected from subject transfected HEK293T cells for downstream binding assay. The CD93-F238L mutant containing the phenylalanine to threonine substitution was generated by PCR using full length CD93 as the template to change the codon sequence from TTC (phenylalanine) to ACC (leucine) (46). All constructs were confirmed by sequencing.

### Flow Cytometry

Cell surface and intercellular staining and analysis by flow cytometry were followed the protocol previously described (71). Dead cells were excluded with SYTOX® (Blue Dead Cell Stain Kit (Thermo Fisher Scientific). Flow cytometric analysis was conducted with a BD FACS Calibur or a BD LSRFortessa™ cell analyzer (BD Bioscience, Franklin Lakes, N.J. USA), and then data were analyzed by FlowJo software (Tree Star Inc.)

### Microscale Thermophoresis (MST) Experiment

IGFBP7 protein (R&D Systems, Minneapolis Minn.) was labeled with a fluorescent dye using a Monolith His-Tag Labeling Kit, RED-tris-NTA 2^{nd} Generation (Nanotemper GMBH, Munchen, Germany). From the 100 nM stock, sample was diluted into PBS—0.05% P20 to a concentration of 20 nM, loaded into Premium MST Capillaries and pretested for successful labeling, and protein stability on a Monolith NT.115 Pico Instrument (Nanotemper GMBH, Munchen, Germany). A stock solution of 5.9 μM recombinant human CD93 protein (R&D Systems, Minneapolis Minn.) was diluted 2-fold 16 times in PBS—0.05% P20 to create a dilution series spanning from 5.9 μM to 180 pM in range. 20 nM IGFBP7 was added to each concentration 1:1 such that each sample contains a final concentration of 10 nM IGFBP7. Samples were loaded into MST Premium Capillaries and measured for microscale thermophoresis on the aforementioned instrument. Experiments were conducted with the PICO Red detector, a laser power of 20% and Medium MST power. This experiment was repeated once with the same procedure for 2 replicates. Data was analyzed using the MO Affinity Analysis software (Nanotemper GMBH, Munchen, Germany).

### EC Culture

Pooled human umbilical vein ECs (HUVEC) purchased from Thermo Fisher were cultured in Medium 200 with LVES (Life Technologies). C57BL/6 mouse primary aortic ECs and the endothelium culture medium with supplement were purchased from Cell Biologics. For tube formation, HUVECs at 2×10⁴ cells/ml were plated on Matrigel in 24-well plate. Image was recorded even 4-6 hours after incubation. The Transwell 6.5 mm polycarbonate membrane inserts pre-loaded in 24-well culture plates (Corning 3422, 8 um) were used in the cell migration model. HUVEC cells at 1×10⁵/ml in 200 μl were loaded into each 24-well insert with 500 μl IBS-containing medium with different reagents in the lower chamber. After approximately 20 hours, the migrated cells were fixed with methanol, stained with Giemsa solution and counted under a light microscope.

### Mouse Tumor Model

All animal care, experiments and euthanasia were performed in accordance with protocols approved by the Institutional Animal Care and Use Committee at the University of Colorado Anschutz Medical Campus. C57BL/6 mice were purchased from the Jackson Laboratory (Bar Harbor, Me.). Mice at 6 to 8 weeks of age were used for these experiments. KPC (4=10⁵) cells were subcutaneously injected into the right flank of C57BL/6 mice. After tumor became palpable, mice were randomised into different treatment groups based on the tumor volume, which was calculated as ½×(length×width²). Therapeutic antibody at 300 μg/mouse was injected intraperitoneally twice a week for total four times. Measurements of tumor diameters (length and width) were taken every 2 or 3 days with a caliper. Mice were euthanized and sacrificed, and tumor tissues were excised for detailed analysis 14 days after first treatment. Tumor tissues for FITC-Lectin, doxorubicin delivery and Hypoxyprobe assay were obtained at day 8 after the first treatment For the combinatory therapy of PD-1 (Clone RMP1-14, BioXcell) and CD93 antibodies. KPC tumor-hearing mice were started with the treatment of antibodies at twice a week for two weeks. Anti-mouse CD4 (Clone GK1.5, BioXcell) or anti-mouse CD8β (Clone 53-5.8, BioXcell) 300 μg/mouse was intraperitoneally administered one da before the first CD93 mAb treatment for CD4/CD8 T cell depletion and repeated at day 7 at a 200 μg dosage. Anti-mouse CD93 mAb treatment was given 300 μg twice a week.

For B16 tumor model, C57BL/6 mice were inoculated subcutaneously with B16 melanoma at 2×1⁵ per mouse. After tumors were detectable, mice were randomized into 4 different groups: control, CD93 mAb alone, 5-FU alone and CD93 mAb+5-FU (combination). CD93 mAb (300 μg i.p.) treatment was administrated on the day of randomization (day 0), day 4 and day 9. 5-FU (3.5 mg i.p.) was administrated on day 2 and day 7. Measurements of tumor size were taken every 2 or 3 days. When tumor volume was exceeding 2000 mm³ and/or ulceration formed, tumor bearing mice were considered as death for the calculation of survival curve.

### Immunohistochemistry and Immunofluorescent Staining

Immunohistochemistry staining protocol has been described previously (72). For immunofluorescent staining, mouse tissue samples were collected and frozen on dry ice using optimum cutting temperature (OTC) mounting fluid. The frozen blocks then were sectioned at 7 μm and mounted on glass slides. The slides were fixed in acetone, blocked with 2.5% goat serum, incubated with primary antibodies for overnight at 4° C., incubated with secondary antibodies for 1 hour, and counterstained with DAPI for 10 min. The slides then were cleared and mounted. Images were taken by Nikon Eclipse TE2000-E upright microscope and analyzed using SlideBook software (Version 6, Intelligent Imaging Inc.) and Image J (Version 1.52K. NIH). Primary antibodies used For IF staining include anti-human IGFBP7 (R115, Sino Biological), anti-human CD31 (JC/70A, ThermoFisher), anti-human CD93 (MM01, Sino Biological), anti-mouse CD3ε (145-2C11), anti-mouse B7-H1 (10F.9G2), anti-mouse IGFBP7 (6F1), and anti-mouse CD93 (7C10). NG2 (Cy3 conjugated pAb, AB5320C3, Millipore) and αSMA (1A4, eFluor 660 Conjugated, Invitrogen) staining was utilized for evaluation of vascular surrounding pericytes. Activated integrin β1 was stained with CD29 mAb (Clone 9EG7) from BD Pharmingen. Ki-67 (16A8, BioLegend) and cleaved caspase 3 (#9661. Cell Signaling) stainings were performed for evaluation of tumor cell proliferation and apoptosis, respectively.

### Hypoxia and Perfusion Measurement

Tumor hypoxia was detected after injection of 30 mg/kg pimonidazole hydrochloride (Hypoxyprobe kit) into tumor-hearing mice (tumors were harvested 1 hour after injection). To detect the formation of pimonidazole adducts tumor frozen sections were stained with APC-Hypoxyprobe mAb following the manufacturers instructions. The hypoxic tumor area was expressed as a percentage of the total tumor area. Drug delivery in tumors was evaluated after tail vein injection of 30 mg/kg Doxorubicin into tumor-bearing mice. Tumors were harvested 1 hour after injection. Doxorubicin on frozen tissue sections was detected by fluorescence microscope with setting of excitation and emission wavelength to 488 and 570 nm. Tumor vessel perfusion was quantified on tumor cryosections following intravenous injection of 50 μg FITC-labeled Lycopersicon esculentum (Tomato) lectin (FL-1171, Vector laboratories, Brussels, Belgium) in tumor-hearing mice (tumors were harvested 10 min after injection). The perfused area was defined as the lectin+ CD31− area expressed as a percentage of the CD31+ area.

### Statistics

Prism software (GraphPad) was used to analyze data and determine statistical significance of differences (including mean±SEM) between groups by apply ink, a 2-tailed, unpaired Student's t test. All P-values less than 0.05 were considered statistically significant.

### REFERENCES

1. Hillen F, and Griffioen A W. Tumour vascularization: sprouting angiogenesis and beyond. Cancer Metastasis Rev. 2007; 26(3-4):489-502
2. De Palma M. Biziato D. and Petrova T V. Microenvironmental regulation of tumour angiogenesis. Nat Rev Cancer. 2017; 17(8):457-74.
3. Carmehet P. and Jain R K. Angiogenesis in cancer and other diseases. Nature. 2000; 407(6801):240-57.
4. McDonald D M, and Baluk P. Significance of blood vessel leakiness in cancer. Cancer Res. 2002; 62(18):5381-5.
5. Dreher M R, Liu W, Michelich C R, Dewhirst M W, Yuan F, and Chilkoti A. Tumor vascular permeability, accumulation, and penetration of macromolecular drug carriers. J Natl Cancer Inst. 2006; 98(5):335-44.
6. Schaaf M B, Garg A D, and Agostinis P. Defining the role of the tumor vasculature in antitumor immunity and immunotherapy. Cell Death Dis. 2018; 9.
7. Quetada S A, Peggs K S, Simpson T R, Shen Y, Littman D R, and Allison J P. Limited tumor infiltration by activated T effector cells restricts the therapeutic activity of regulatory T cell depletion against established melanoma. J Exp Med. 2008; 205(9):2125-38.
8. Buckanovich R J, Facciabene A, Kim S, Benencia F, Sasaroli D, Balint K, et al. Endothelin B receptor mediates the endothelial harrier to T cell homing to tumors and disables immune therapy. Nature Medicine. 2008; 14(1):28-36.
9. Hatfield S M, Kjaergaard J, Lukashev D, Schreiber T H, Belikoff B, Abbott R, et al. Immunological mechanisms of the antitumor effects of supplemental oxygenation. Sci Transl Med. 2015; 7(277).
10. Dang E V, Barbi J. Yang H Y, Jinasena D, Yu H, Zheng Y, et al. Control of T(H)17/T-reg Balance by Hypoxia-Inducible Factor I. Cell. 2011; 146(5):772-84.
11. Yan M, Jene N, Byrne D, Millar E K A, O'Toole S A. McNeil C M, et al. Recruitment of regulatory T cells is correlated with hypoxia-induced CXCR4 expression, and is associated with poor prognosis in basal-like breast cancers, Breast Cancer Res. 2011; 13(2).
12. Chanmee T, Ontong P, Konno K, and Itano N. Tumor-Associated Macrophages as Major Players in the Tumor Microenvironment. Cancers. 2014; 6(3):1670-90.
13. Ceradini D J, Kulkarni A R, Callaghan M J, Tepper O M, Bastidas N. Kleinman M E, et al. Progenitor cell trafficking is regulated by hypoxic gradients through HIF-1 induction of SDF-1. Nat Med. 2004; 10(8):838-64.
14. Ranieri G, Patruno R, Ruggieri F., Montemurro S, Valerio P, and Ribatti D. Vascular endothelial growth factor (VEGF) as a target of bevacizumab in cancer: from the biology to the clinic. Curr Med Chem. 2006; 13(16):1845-57.
15. Hurwitz H, Fehrenbacher L, Novotny W, Cartwright T, Hainsworth J, Heim W, et al. Bevacizumab plus irinotecan, fluorouracil, and leucovorin for metastatic colorectal cancer. N Engl J Med. 2004; 350(23):2335-42.
16. Huang Y, Yuan J, Righi E, Kamoun W S, Ancukiewicz M, Nezivar J, et al. Vascular normalizing doses of antiangiogenic treatment reprogram the immunosuppressive tumor microenvironment and enhance immunotherapy. Proc Natl Acad Sci USA. 2012; 109(43):17561-6.
17. Huang Y, Stylianopoulos T, Duda D G, Fukumura D, and Jain R K. Benefits of vascular normalization are dose and time dependent-letter. Cancer Res. 2013; 73(23):7144-6.
18. Jain R K. Normalization of tumor vasculature: an emerging concept in antiangiogenic therapy. Science. 2005; 307(5706):58-62.
19. Hamzah J, Jugold M, Kiessling F, Rigby P, Mansur M, Marti H H, et al. Vascular normalization in Rgs5-deficient tumours promotes immune destruction. Nature. 2008; 453(7193):410-4.
20. Huang Y H, Goel S, Duda D G, Fukumura D, and Jain R K. Vascular Normalization as an Emerging Strategy to Enhance Cancer Immunotherapy. Cancer Research. 2013; 73(10):2943-8.
21. Zheng X C, Fang Z X, Liu X M, Deng S M, Zhou P, Wang X X, et al. Increased vessel perfusion predicts the efficacy of immune checkpoint blockade. Journal of Clinical Investigation. 2018; 128(5):2104-15.
22. Huang Y H, Kim B Y S, Chan C K, Hahn S M, Weissman I L, and Jiang W. Improving immune-vascular crosstalk for cancer immunotherapy. Nat Rev Immunol. 2018; 18(3):195-203.
23. Fukumura D, Kloepper J, Amoozgar Z, Duda D G, and Jain R K. Enhancing cancer immunotherapy using antiangiogenics: opportunities and challenges. Nat Rev Clin Oncol. 2018; 15(5):325-40.
24. Makker V, Rasco D W, Vogelzang N J, Messing M, Brose M S, and Cohn A L. Lenvatinib plus pembrolizumab in patients with advanced endometrial cancer: Updated results. Journal of Clinical Oncology. 2018; 36(15).
25. Motzer R J, Powles T, Atkins M B, Escudier B, McDermott D F, Suarez C, et al. IMmotion151: A Randomized Phase III Study of Atezolizumab Plus Bevacizumab vs Sunitinib in Untreated Metastatic Renal Cell Carcinoma (mRCC). Journal of Clinical Oncology. 2018; 36(6).
26. Socinski M A, Jotte R M, Cappuzzo F, Orlandi F, Stroyakovskiy D, Nogami N, et al. Atezolizumab for First-Line Treatment of Metastatic Nonsduamous NSCLC. New Engl J Med 2018; 378(24):2288-301.
27. Apte R S, Chen D S, and Ferrara N. VEGF in Signaling and Disease Beyond Discovery and Development. Cell. 2019; 176(6):1248-64.
28. Masiero M, Simocs F C, Han H D, Snell C, Peterkin T, Bridges E, et al. A Core Human Primary Tumor Angiogenesis Signature Identifies the Endothelial Orphan Receptor ELTD1 as a Key Regulator of Angiogenesis. Cancer Cell. 2013; 24(2):229-41.
29. Brauer M J, Zhuang G L, Schmidt M, Yao J, Wu X M, Kaminker J S, et al. Identification and Analysis of In vivo VEGF Downstream Markers Link VEGF Pathway Activity with Efficacy of Anti-VEGF therapies Clin Cancer Res 2013; 19(13):3681-92.
30. Baker L C J, Boult J K R, Thomas M, Koehler A, Nayak T, Tessier J, et al. Acute tumour response to a bispecific Ang-2-VEGF-A antibody: insights from multiparametric MRI and gene expression profiling. Brit J Cancer. 2016; 115(6):691-702.
31. Bais C, Singh M, Kaminker J, and Brauer M. Google Patents; 2011.
32. Olsen R S. Lindh M, Vorkapic E, Andersson R E, Zar N, Lofgren S, et al. CD93 gene polymorphism is associated with disseminated colorectal cancer. Int J Colorectal Dis. 2015; 30(7):883-90.
33. Langenkamp E, Zhang L, Lugano R, Huang H, Abu Elhassan T E, Georganaki M, et al. Elevated Expression of the C-Type Lectin CD93 in the Glioblastoma Vasculature Regulates Cytoskeletal Rearrangements That Enhance Vessel Function and Reduce Host Survival. Cancer Research. 2015; 75(21):4504-16.
34. Ban L L, Tang M M, Zhang Q C, You B, Shan Y, Shi S, et al. Elevated expression of CD93 promotes angiogenesis and tumor growth in nasopharyngcal carcinoma. Biochem Bioph Res Co. 2016; 476(4):467-74.
35. Galvagni F, Nardi F, Spiga O, Treiza A, Tarticchio G, Pellicani R, et al. Dissecting the CD93-Multimerin 2 interaction involved in cell adhesion and migration of the activated endothelium. Matrix Biology. 2017; 64:112-27.
36. Hingorani S R, Wang L, Multani A S, Combs C, Deramaudt T B, Hruban, R H, et al. Trp53R172H and KrasG12D cooperate to promote chromosomal instability and widely metastatic pancreatic ductal adenocarcinoma in mice. Cancer Cell. 2005; 7(5):469-83.
37. Bergers G, and Song S. The role of pericytes in blood-vessel formation and maintenance. Neuro Oncol. 2005; 7(4):452-64.
38. Herbert S P, and Stainier D Y. Molecular control of EC behaviour during blood vessel morphogenesis. Nat Rev Mol Cell Biol. 2011; 12(9) 551-64.
39. Gaengel K, Genove G, Armulik A, and Betsholtz C. Endothelial-mural cell signaling in vascular development and angiogenesis. Arterioscler Thromb Vase Biol. 2009; 29(5):630-8.
40. Schmittnaegel M, Rigamonti N, Kadioglu E, Cassara A, Rmili C W, Kiialainen A, et al. Dual angiopoietin-2 and VEGFA inhibition elicits antitumor immunity that is enhanced by PD-1 checkpoint blockade. Sci Transl Med. 2017; 9(385).
41. Hamzah J, Jugold M, Kiessling F, Rigby P, Manzur M, Marti H H, et al. Vascular normalization in Rgs5-deficient tumours promotes immune destruction. Nature. 2008; 453(7193):410-U67.
42. Wang J, Sanmamed M F, Datar I, Su T T, Sun J, et al. Fibrinogen-like Protein 11s a Major Immune Inhibitory Ligand of LAG-3. Cell. 2019; 176(1-2):334-47 e12.
43. Zhu S, Xu F, Zhang J, Ruan W, and Lai M. Insulin-like growth factor binding protein-related protein 1 and cancer. Clin Chim Acta. 2014; 431:23-32.
44. Cai Z, Chen H T, Boyle B, Rupp F, Funk W D, and Dedera D A. Identification of a novel insulin-like growth factor binding protein gene homologue with tumor suppressor like properties. Biochem Biophys Res Commun. 2005; 331(1):261-6.
45. Khan K A, Naylor A J, Khan A, Noy P J, Mambretti M, Lodhia P, et al. Multimerin-2 is a ligand for group 14 family C-type lectins CLEC14A, CD93 and CD248 spanning the endothelial pericyte interface. Oncogene. 2017; 36(44):6097-108.
46. Galvagni F, Nardi F, Spiga O, Trezza A, Tarticchio G, Pellicani R, et al. Dissecting the CD93-Multimerin 2 interaction involved in cell adhesion and migration of the activated endothelium. Matrix Biol. 2017; 64:112-27.
47. Schodel J, Oikonomopoulos S, Ragoussis J. Pugh C W, Ratcliffe P J, and Mole D R, High-resolution genome-wide mapping of HIF-binding sites by ChIP-seq. Blood. 2011; 117(23):E207-E17.
48. Komiya F, Sato H, Watanabe N, Ise M, Higashi S. Miyagi Y, et al. Angiomodulin, a marker of cancer vasculature is upregulated by vascular endothelial growth factor and increases vascular permeability as a ligand of integrin alphavbeta3. Cancer Med. 2014; 3(3):537-49.
49. Zhao Q, Eichten A, Parveen A, Adler C, Huang Y, Wang W, et al. Single-Cell Transcriptome Analyses Reveal E C Heterogeneity in Tumors and Changes following Antiangiogenic Treatment. Cancer Research. 2018; 78(9):2370-82.
50. Hooper A T, Shmelkov S V, Gupta S, Milde T, Bambino K, Gillen K, et al. Angiomodulin is a specific marker of vasculature and regulates vascular endothelial growth factor-A-dependent neoangiogenesis. Circ Res. 2009; 105(2):201-8.
51. Hakanpaa L, Sipila T, Leppanen V M, Gautam P, Nurmi H, Jacquemet G, et al. Endothelial destabilization by angiopoietin-2 via integrin beta1 activation. Nat Commun. 2015; 6:5962.
52. Taupe J M, Anders R A, Young G D, Xu H Y, Sharma R, McMiller T L, et al. Colocalization of Inflammatory Response with B7-H1 Expression in Human Melanocytic Lesions Supports an Adaptive Resistance Mechanism of Immune Escape. Sci Transl Med. 2012; 4(127).
52′ Dong et al, Nature Med 2002
53. Meadows K L, and Hurwitz H I. Anti-VEGF Therapies in the Clinic. Csh Perspect Med. 2012; 2(10).
54. Huang Y H, Yuan J P, Righi E, Kamoun W S, Ancukiewicz M. Nezivar J, et al. Vascular normalizing doses of antiangiogenic treatment reprogram the immunosuppressive tumor microenvironment and enhance immunotherapy. P Natl Acad Sci USA. 2012; 109(43):17561-6.
55. Akiel M, Guo C, Li X, Rajasekaran D, Mendoza R G, Robertson C L, et al. IGFBP7 Deletion Promotes Hepatocellular Carcinoma. Cancer Res. 2017; 77(15):4014-25.
56. Norsworthy P J, Fossati-Jimack L, Cortes-Hernandez J, Taylor P R, Bygrave A E, Thompson R D, et al. Murine CD93 (C1qRp) contributes to the removal of apoptotic cells in vivo but is not required for C1q-mediated enhancement of phagocytosis. Journal of Immunology. 2004; 172(6):3406-14.
57. Hwa V, Oh Y, and Rosenfeld R G. The insulin-like growth factor-binding protein (IGFRP) superfamily. Endocr Rev. 1999; 20(6):761-87.
58. Bach L A. What Happened to the IGF Binding Proteins? Endocrinology. 2018; 159(2):570-8.
59. Oh Y, Nagalla S R, Yamanaka Y, Kim H S, Wilson F, and Rosenfeld R G. Synthesis and characterization of insulin-like growth factor-binding protein (IGFBP)-7. Recombinant human mac25 protein specifically binds IGF-I and -II. J Biol Chem. 1996; 271(48):30322-5.
60. Wajapeyee N, Serra R W, Zhu X, Mahalingam M, and Green M R. Oncogenic BRAF induces senescence and apoptosis through pathways mediated by the secreted protein IGFBP7. Cell. 2008; 132(3):363-74.
61. Cao Z., Scandura J M, Inghirami G G, Shido K, Ding B S, and Rafii S. Molecular Checkpoint Decisions Made by Subverted Vascular Niche Transform Indolent Tumor Cells into Chemoresistant Cancer Stem Cells. Cancer Cell 2017; 31(1):110-26.
62. Evdokimoa V, Tognon C E, Benatar T, Yang W, Krutikov K, Pollak M, et al. IGFBP7 binds to the IGF-1 receptor and blocks its activation by insulin-like growth factors. Sci Signal. 2012; 5(255):ra92.
63. Darr J, Klochendler A, Isaac S, and Eden A. Loss of IGFBP7 expression and persistent AKT activation contribute to SMARCB1/Snf5-mediated tumorigenesis. Oncogene. 2014; 33(23):3024-32.
64. Pen A, Moreno M J, Durocher Y, Deb-Rinker P, and Stanimirovic D B. Glioblastoma-secreted factors induce IGFBP7 and angiogenesis by modulating Smad-2-dependent TGF-beta signaling. Oncogene. 2008; 27(54):6834-44.
65. Akaogi K, Okahe Y, Sato J, Nagashima Y, Yasumitsu H, Sugahara K, et al. Specific accumulation of tumor-derived adhesion factor in tumor blood vessels and in capillary tube-like structures of cultured vascular ECs. Proc Natl Acad Sci USA. 1996; 93(16):8384-9.
66. Sato J, Hasegawa S, Akaogi K, Yasumitsu H, Yamada S, Sugahara K, et al. Identification of cell-binding site of angiomodulin (AGM/TAF/Mac25) that interacts with heparan sulfates on cell surface. J Cell Biochem. 1999; 75(2):187-95.
67. Mura M. Swain R K, Zhuang X, Vorschmitt H, Reynolds G, Durant S, et al. Identification and angiogenic role of the novel tumor endothelial marker CLEC14A. Oncogene. 2012; 31(3):293-305.
68. Noy P J, Lodhia P, Khan K, Muting X, Ward D G, Verissimo A R, et al. Blocking CLEC14A-MMRN2 binding inhibits sprouting angiogenesis and tumour growth. Oncogene. 2015; 34(47):5821-31.
69. Lugano R, Venturi K, Yu D, Bergqvist M, Smits A, Essand M, et al. CD93 promotes beta1 integrin activation and fibronectin fibrillogenesis during tumor angiogenesis. J Clin Invest. 2018; 128(8):3280-97.
70. Langenkamp E, Zhang L, Lugano R, Huang H, Elhassan T E, Georganaki M, et al. Elevated expression of the C-type lectin CD93 in the glioblastoma vasculature regulates cytoskeletal rearrangements that enhance vessel function and reduce host survival. Cancer Res. 2015; 75(21):4504-16.
71. Tian Y, Sun Y, Gao F, Koenig M R, Sunderland A, Fujiwara Y, et al. CD28H expression identifies resident memory CD8−T cells with less cytotoxicity in human peripheral tissues and cancers. Oncoimmunology. 2019; 8(2).
72. Zhu Y W, Yao S, Iliopoulou B P, Han X, Augustine M M, Xu H Y, et al. B7-H5 costimulates human T cells via CD28H. Nature Communications. 2013; 4.
73. Greenlee M C, Sullivan S A, Bohlson S S, CD93 and related family members: Their role in innate immunity. Curr Drug Targets. 2008; 9(2):130-8. PubMed PMID: WOS:000253937200005.
74. Shrimali R K, Yu Z Y, Theoret M R, Chinnasamy D, Reslifo N P, Rosenberg S A. Antiangiogenic Agents Can Increase Lymphocyte Infiltration into Tumor and Enhance the Effectiveness of Adoptive Immunotherapy of Cancer. Cancer Research. 2010; 70(15):6171-80. doi: 10.1158/0008-5472.Can-10-0153. PubMed PMID: WOS:000280557500007.
75. Zhu Y W, Yao S. Augustine M M, Xu H Y, Wang J. Sun J W, Broadwater M, Ruff W, Luo L Q, Zhu G F. Yamada K, Chen L P. Neuron-specific SALM5 limits inflammation in the CNS via its interaction with HVEM. Sci Adv. 2016; 2(4), doi: UNSP e1500637 10.1126/sciadv. 1500637. PubMed PMID: WOS:000380072100002
76. Fujiwara Y, Sun Y, Torphy R J, He J D, Yanaga K, Edil B H, Schulick R D, Zhu Y W, Pomalidomide Inhibits PD-L1 Induction to Promote Antitumor Immunity. Cancer Research. 2018; 78(23):6655-65. doi: 10.1158/0008-5472.Can-18-1781. PubMed PMID: WOS:000452360300013.
77. Mariathasan, S., Purley, S. J., Nickles, D., Castiglioni, A., Yuen, K., Wang, Y. L., Kadel, E. E., Koeppen, H., Aslarita, J. L., Cubas, R., et al. (2018). TGF beta attenuates tumour response to PD-L1 blockade by contributing to exclusion of T cells. Nature 554, 544-548.
78. Hugo, W., Zaretsky, J. M., Sun, L., Song, C. Y., Moreno, B. H., Hu-Lieskovan, S., Berent-Maoz, B., Pang, J., Chmielowski, B., Cherry, G., et al. (2017). Genomic and Transcriptomic Features of Response to Anti-PD-1 Therapy in Metastatic Melanoma (vol 165, pg 35, 2016). Cell 168, 542-542.
79. Yao S, Zhu Y, Zhu G, Augustine M, Zheng L, Goode D J, B7-H2 IS A COSTIMULATORY LIGAND FOR CD28 IN HUMAN. Immunity. 2011 May 27; 34(5): 720-740.

SEQUENCE TABLE

| SEQ | | |
|---|---|---|
| ID | | |
| NO | Description | Sequences |
| 1 | Human | MATSMGLLLLLLLLLTQPGA |
| | CD93 | GTGADTEAVVCVGTACYTAH |
| | | SGKLSAAEAQNHCNQNGGNL |
| | | ATVKSKEEAQHVQRVLAQLL |
| | | RREAALTARMSKFWIGLQRE |
| | | KGKCLDPSLPLKGFSWVGGG |
| | | EDTPYSNWHKELRNSCISKR |
| | | CVSLLLDLSQPLLPSRLPKW |
| | | SEGPCGSPGSPGSNIEGFVC |
| | | KFSFKGMCRPLALGGPGQVT |
| | | YTTPFQTTSSSLEAVPFASA |
| | | ANVACGEGDKDKETQSHYFL |
| | | CKEKAPDVFDWGSSGPLCVS |
| | | PKYGCNFNNGGCHQDCFEGG |
| | | DGSFLCGCRPGFRLLDDLVT |
| | | CASRNPCSSSPCRGGATCVL |
| | | GPHGKNYTCRCPQGYQLDSS |
| | | QLDCVDVDECQDSPCAQECV |
| | | NTCPGGFRCECWVGYKPGGP |
| | | GEGACQDVDECALGRSPCAQ |
| | | GCTNTDGSFHCSCEEGYVLA |
| | | GEDGTQCQDVDECVGPGGPL |
| | | CDSLCFNTQGSFHCGCLPGW |
| | | VLAPNGVSCTMGPVSLGPPS |
| | | GPPDEEDKGEKEGSTVPRAA |
| | | TASPTRGPEGTPKATPTTSR |
| | | PSLSSDAPfTSAPLKMLAPS |
| | | GSPGVWREPSIHHATAASGP |
| | | QEPAGGDSSVATQNNDGTDG |
| | | QKLLLFYILGTVVAILLLLA |
| | | LALGLLVYRKRRAKREEKKE |
| | | KKPQNAADSYSWVPERAESR |
| | | AMENQYSPTPGTDC |
| 2 | Human | MERPSLRALLLGAAGLLLLL |
| | IGFBP7 | LPLSSSSSSDTCGPCEPASC |
| | | PPLPPLGCLLGKTRDACGCC |
| | | PMCARGEGEPCGGGGAGRGY |
| | | CAPGMECVKSRKRRKGKAGA |
| | | AAGGPGVSGVCVCKSRYPVC |
| | | GSDGTTYPSGCQLRAASQRA |
| | | ESRGEKAITQVSKGTCEQGP |
| | | SIVTPPKDIWNVTGAQVYLS |
| | | CEVIGIPTPVLIWNKVKRGH |
| | | YGVQRTELLPGDRDNLAIQT |
| | | RGGPEKHEVTGWVLVSPLSK |
| | | EDAGEYECHASNSQGQASAS |
| | | AKITVVDALHEIPVKKGEGA |
| | | EL |

The claimed subject matter is not to be limited in scope by the specific embodiments described herein. Indeed various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating a tumor or cancer in a subject in need thereof, comprising administering to the subject an effective amount of a CD93/IGFBP7 blocking agent that specifically inhibits the IGFBP7/CD93 signaling pathway.

2. The method of claim 1, wherein the CD93/IGFBP7 blocking agent blocks interaction between CD93 and IGFBP7.

3. The method of claim 2, wherein the CD93/IGFBP7 blocking agent comprises an anti-CD93 antibody specifically recognizing CD93.

4-5. (canceled)

6. The method of claim 3, wherein the anti-CD93 antibody also blocks interaction between CD93 and MMRN2.

7. The method of claim 3, wherein the anti-CD93 antibody does not block interaction between CD93 and MMRN2.

8-19. (canceled)

20. The method of claim 3, wherein the anti-CD93 antibody is a full length antibody, a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, or a tetrabody.

21. The method of claim 3, wherein the anti-CD93 antibody is comprised in a fusion protein.

22-32. (canceled)

33. The method of claim 2, wherein the CD93/IGFBP7 blocking agent comprises an anti-IGFBP7 antibody specifically recognizing IGFBP7.

34-48. (canceled)

49. The method of claim 33, wherein the anti-IGFBP7 antibody is a full length antibody, a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, or a tetrabody.

50-61. (canceled)

62. The method of claim 1, further comprising administering to the subject a second agent.

63. The method of claim 62, wherein the second agent is an immune checkpoint inhibitor.

64. The method of claim 63, wherein the immune checkpoint inhibitor is an anti-PD1 antibody, an anti-PD-L1 antibody, an anti-CTLA4 antibody, or a combination thereof.

65. The method of claim 62, wherein the second agent is a chemotherapeutic agent.

66. The method of claim 62, wherein the second agent is an immune cell.

67. The method of claim 62, wherein the second agent is an anti-angiogenesis inhibitor.

68. The method of claim 67, wherein the anti-angiogenesis inhibitor is an anti-VEGF inhibitor.

69. The method of claim 1, wherein the cancer is characterized by abnormal tumor vasculature.

70. (canceled)

71. The method of claim 1, wherein the cancer is characterized by high expression of CD93.

72. The method of claim 1, wherein the cancer is characterized by high expression of IGFBP7.

73. The method of claim 1, wherein the cancer is a solid tumor.

74. The method of claim 73, wherein the cancer is colorectal cancer, non-small cell lung cancer, glioblastoma, renal cell carcinoma, cervical cancer, ovarian cancer, fallopian tube cancer, peritoneal cancer, breast cancer, prostate cancer, bladder cancer, oral squamous cell carcinoma, head and neck squamous cell carcinoma, brain tumors, bone cancer, melanoma.

75. The method of claim 74, wherein the cancer is triple-negative breast cancer (TNBC).

76. The method of claim 1, wherein the cancer is enriched with blood vessels.

77. A method of determining whether a candidate agent is useful for treating cancer, comprising: determining whether the candidate agent disrupts the CD93/IGFBP7 interaction, wherein the candidate agent is useful for treating cancer if it is shown to specifically disrupt the CD93/IGFBP7 interaction.

78-84. (canceled)

85. An agent identified by the method of claim 77.

86. A non-naturally occurring polypeptide which is a variant inhibitory CD93 polypeptide comprising the extracellular domain of CD93, wherein the polypeptide blocks interaction between CD93 and IGFBP7.

87-94. (canceled)

95. A non-naturally occurring variant inhibitory IGFBP7 polypeptide comprising a variant of IGFBP7, wherein the polypeptide blocks interaction between CD93 and IGFBP7.

96-102. (canceled)

103. A pharmaceutical composition comprising i) the agent of claim 85, ii) a non-naturally occurring polypeptide which is a variant inhibitory CD93 polypeptide comprising the extracellular domain of CD93, wherein the polypeptide blocks interaction between CD93 and IGFBP7, or iii) a non-naturally occurring variant inhibitory IGFBP7 polypeptide comprising a variant of IGFBP7, wherein the polypeptide blocks interaction between CD93 and IGFBP7, and a pharmaceutically acceptable carrier and/or excipient.
